(19) United States
(12) Patent Application Publication
(10) Pub. No.: US 2009/0246349 A1
(43) Pub. Date: Oct. 1, 2009

(54) Wheat pigment

(76) Inventors: Joanna Louise Mimica, New South Wales (AU); Crispin Alexander Howitt, ACT (AU); Barry James Pogson, ACT (AU)

Correspondence Address: COOPER & DUNHAM, LLP, 30 Rockefeller Plaza, 20th Floor, NEW YORK, NY, 10112, US

(21) Appl. No.: 11/988,768

(22) PCT Filed: Jul. 11, 2006
(86) PCT No.: PCT/AU2006/000979

§ 371 Date: Feb. 27, 2009

Related U.S. Application Data
(60) Provisional application No. 60/698,518, filed on Jul. 11, 2005.

(51) Int. Cl. A21D 2/00; A01H 5/00; C12N 15/82; C12N 9/00; C07H 21/04; C07H 21/02; C12N 5/10; C12Q 1/68; A01G 1/00; A01D 45/00; A01D 45/30

(52) U.S. Cl. 426/622; 800/298; 800/282; 800/320; 800/320.3; 435/183; 536/23.2; 536/23.6; 536/24.5; 435/419; 435/6; 426/443; 47/58.1 FV; 56/126

(57) ABSTRACT

The present invention relates to genes, and markers therefor, involved in lutein production in cereal plants such as wheat. In particular, the invention characterizes ε-cyclase and phytoene synthase genes from wheat. Furthermore, the present invention relates to the manipulation of ε-cyclase and/or phytoene synthase expression to alter colour traits of a plant which can effect the characteristics of food and/or non-food products obtained from said plants.

### FIELD OF THE INVENTION

The present invention relates to genes, and markers therefor, involved in lutein production in cereal plants such as wheat. In particular, the invention characterizes ε-cyclase and phytoene synthase genes from wheat. Furthermore, the present invention relates to the manipulation of ε-cyclase and/or phytoene synthase expression to alter colour traits of a plant which can effect the characteristics of food and/or non-food products obtained from said plants.

### BACKGROUND OF THE INVENTION

Grain colour in cereals such as wheat is an important quality trait, strongly influencing the colour of flour produced from the grain. Grain colour has long been a dependable and valuable factor for evaluating wheat flour quality (Lepage and Sims, 1968; Johnston et al., 1980) and different grades of wheat have specific colour requirements. Samples collected at the completion of harvest are tested for basic quality parameters including colour as well as milling performance and end product suitability. Minimum pigmentation is sought in wheat used for breads, biscuits, cakes, and pastry purposes where it is desired that the flour be as white as possible. In contrast, amber coloured kernels are more desired for durum wheat where high levels of stable yellow pigment are ideally suited to semolina or pasta production. For wheat intended for noodle production, there are three noodle types marketed, fresh yellow alkaline noodles (YAN), Japanese white udon noodles and instant noodles. Production of a noticeable yellow colour is desired for YAN when the flour is reacted with alkali.

There are at least three factors that affect the flour colour of wheat samples. The presence of bran flakes gives speckiness to the flour and is due to the milling and sieving procedures used during the separation of the outer seed coat from the endosperm. Polyphenol oxidase (PPO) in wheat flour catalyses the oxidation of phenolic compounds, which can result in a darkening of flour samples after milling and exposure to oxygen. However, the actual colour of the grain is the primary determinant of flour colour (Mares and Campbell, 2001). The colour of the grain is due mainly to the presence of two naturally occurring yellow pigment classes, carotenoids and flavonoids.

Flavonoids are a class of pigments that are abundant in most seeds and include flavonols, anthocyanins (red, blue or purple pigments), proanthocyanidins (brown), 3-deoxy-anthocyanidins and isoflavonoids. Flavonoids accumulate in the embryo of all plants. In wheat grain, the flavonoids include 6,8-di-C-glycosylapigenins, present in the germ and bran but not the endosperm. These are colourless at low or neutral pH but are bright yellow at alkaline pH and therefore visible in wheat flour after processing with alkali. Flavonoids contribute ⅔ to the total yellowness of YAN while the remainder is due to carotenoids (Mares et al., 1997).

Carotenoids, which are C₄₀ isoprenoids comprised of eight isoprene units, are the second most abundant type of pigment in nature and include over 600 different compounds (Britton et al., 1998). Carotenoids accumulate to high levels in many fruits and flowers and are responsible for the bright red colour of tomato, the orange of carrots and the yellow of marigolds, to name a few examples (Cuttriss and Pogson, 2004). Carotenoids are also responsible for the yellow pigmentation of untreated wheat grain. Since the flavonoids of wheat grain are colourless unless treated with alkali, carotenoids are the primary determinant of yellow pigment content of untreated or unbleached wheat flours and have greater implications on the marketing of wheat.

Carotenoids have a polyene chain consisting of 3-15 conjugated double bonds which is responsible for the characteristic absorption spectrum of each of the compounds and therefore the colour and other properties. They include the non-oxygenated carotenes which may be linear or cyclized, and xanthophylls which are formed by the introduction of oxygen functions to carotenes. The carotenoid backbone is either linear or contains cyclic end groups. The most abundant end group is the β-ionone ring of β-carotene and its derivatives (β,β-xanthophylls). Other cyclic end groups are the ε-ring found in lutein and α-carotene (Cunningham and Gantt, 1998). The most commonly occurring carotenes are β-carotene, which accumulates in chloroplasts, and lycopene, which accumulates in some fruits such as tomatoes. The most abundant xanthophylls are lutein, violaxanthin and neoxanthin, which are key components of the light-harvesting complex of leaves and photosynthesis. Carotenoids in plants are reviewed by Cuttriss et al. (2005).

The only carotenoid conclusively detected in wheat flour is lutein (Lepage and Sims, 1968; Kaneko et al., 1995; Hentschel et al., 2002). Lutein is a xanthophyll product of the carotenoid biosynthesis pathway (see below). Lutein is distributed throughout all milling fractions including the endosperm and therefore the flour. Some studies have claimed that the identification of carotenoids in wheat flour that contribute to the absorbance at 440 nm is incomplete. Five individual peaks, corresponding to potentially five different carotenoid pigments, were identified in three different wheat flours by Ward et al. (1997). Lutein was the major pigment present and one peak was identified as β-carotene, however the three other peaks could not be identified. Hentschel et al. (2002) performed HPLC analysis of flour carotenoids and reported the sole presence of lutein in eight durum wheat cultivars. Quantification of the lutein quantified by HPLC fell short of total carotenoid content as quantified by spectrophotometry and therefore it was claimed that HPLC did not reveal all of the grain pigments (Hentschel et al., 2002). In another study, the yellowness of wheat flour was attributed to the presence of β-carotene (Santra et al., 2003). Generally, the role of carotenoids as determinants of flour colour in wheat is not well characterised.

### Biosynthesis of Carotenoids

The biosynthesis of carotenoids is shown schematically in FIG. 1. The first committed step to carotenoids is the condensation of two molecules of geranylgeranyl pyrophosphate to form the colourless carotenoid phytoene, which does not usually accumulate in tissues. This reaction is catalysed by the enzyme phytoene synthase (PSY) (Harker and Hirschberg, 1998). In plants, four de-saturation reactions then convert phytoene into the red coloured compound lycopene, catalysed by two enzymes, phytoene desaturase (PDS) and δ-carotene desaturase. The production of all-trans-lycopene, which is the preferred substrate for the following cyclases, also requires the carotenoid isomerase, CRTISO (Park et al., 2002).

The ends of the linear carotenoid lycopene can be cyclized and modified with oxygen to form the xanthophylls (Cuttriss and Pogson, 2004). Cyclisation of lycopene is a branch point in the pathway, where one pathway leads to β-carotene and the other to α-carotene. A single enzyme called lycopene β-cyclase catalyses the formation of β-carotene by introducing two rings onto either end of lycopene. In the other branch of the pathway, two enzymes called lycopene ε-cyclase and lycopene β-cyclase convert lycopene to α-carotene. The ε-cyclase introduces one ring to the end of lycopene, to form δ-carotene before an additional ring is added to the other end by β-cyclase to form α-carotene (Cuttriss and Pogson, 2004).

β-cyclase was first identified in cyanobacteriurn (Cunningham et al., 1993; Cunningham et al., 1994) and has since been characterised in higher plants. In Arabidopsis β-cyclase mutants are lethal indicating lack of a redundant gene with β-cyclase function. However, a second β-cyclase in tomato has been identified, which is important in pigment accumulation, although sequence similarity to the classical β-cyclase is only 53% (Ronen et al., 2000). In higher plants, β-cyclase can usually only catalyse cyclisation of one end group of lycopene and β-cyclase is required to add a second ring to form α-carotene (Cuttriss and Pogson, 2004). In an exception, the ε-cyclase in lettuce functions to catalyse formation of a bicyclic ε,ε-carotene (Cunningham and Gannt, 2001; Phillip and Young, 1995). The unusual function of lettuce ε-cyclase is due to a single amino acid addition which can be mutated to reduce ε-cyclase function back to one ε-ring addition only (Cunningham and Gannt, 2001). A novel cyclase has also been identified in the marine cyanobacterium Prochlorococcus marinus MED4 which can form both β and ε rings on lycopene (Stickforth et al., 2003) but to date both β-cyclase and ε-cyclase are required for α-carotene production in higher plants (Pogson et al., 1996).

α-carotene and β-carotene may be further modified to produce xanthophylls, which are enzymatically formed oxidation products (Cuttriss and Pogson, 2004). The xanthophylls typically have either a hydroxy group at C-3 or an epoxy at the 5,6-position of the ionone ring (Cuttriss and Pogson, 2004). Two different hydroxylases, which are specific for β- and ε-ring formation, add hydroxy groups (Pogson et al., 1996; Hirschberg, 2001).

Lutein is a 3,3′-dihydroxy-α-carotene. Zeaxanthin, on the alternate branch of the pathway, is a 3,3′-dihydroxy-β-carotene. Lutein is the end product of the α-carotene branch whereas zeaxanthin, on the β-carotene branch, can be further modified by epoxidation (Cuttriss and Pogson, 2004). Epoxidation involves the addition of oxygen at the 5,6-position of the ionone ring. Epoxidation of zeaxanthin to violaxanthin is part of a reversible cycle and under light stress, de-epoxidation of violaxanthin occurs via antheraxanthin, back to zeaxanthin (Pfundel and Bilger, 1994). It is thought that high levels of zeaxanthin are necessary for the protection of the chloroplast against high light conditions, favouring the reaction (Cuttriss and Pogson, 2004). Violaxanthin may be converted to neoxanthin by a neoxanthin synthase enzyme (Bouvier et al., 2000). Neoxanthin is the last carotenoid of the β-carotene branch of the carotenoid pathway in higher plants.

An important following reaction in the β-carotene pathway is the conversion of some of the products to hormones such as abscisic acid (ABA). Both neoxanthin and violaxanthin can be cleaved by 9-cis-epoxycarotenoid dioxygenase (NCED) and these cleavage products are further modified to produce ABA (Schwartz et al., 1997; Seo and Koshiba, 2002). The link between carotenoid and ABA levels is shown in viviparous maize mutants which display precocious seed germination due to ABA deficiency and contain decreased levels of carotenoids (Maluf et al., 1997). In a tissue such as wheat grain, however, where low levels of carotenoids are present and only trace levels of β,β-xanthophylls are present, carotenoid biosynthesis genes could also regulate ABA synthesis as a consequence of carotenoids feeding through to ABA synthesis and accumulation.

### Functions of Carotenoids

Carotenoids are essential in photosynthesis and also serve as photoprotective compounds by quenching triplet chlorophyll and singlet oxygen derived from excess light energy (Demmig-Adams and Adams, 1996; Cuttriss et al., 2005). Carotenoids also function as photoprotective compounds in fruits and flowers. The role of carotenoids in seed is less clear. It is known that carotenoid production in the seed is important for ABA production and seed dormancy (Maluf et al., 1997). Furthermore, carotenoids contribute to the antioxidant system in seeds, which has protective functions against free radicals, membrane deterioration and seed aging (Pinzino et al., 1999; Calucci et al., 2004). In wheat seed, carotenoids have been viewed as important antioxidants that limit the levels of free radicals and reduce peroxidase activity, which may protect against seed ageing (Pinzino et al., 1999; Calucci et al., 2004). Lipid peroxidation of membranes leads to membrane deterioration, which is connected with seed ageing and loss of seed viability (Pinzino et al., 1999). During seed germination, higher antioxidant contents are correlated with an inhibition of peroxidase activity and promote seed germination (Rogozhin et al, 2001).

Carotenoids are also vital dietary compounds for human health (Cuttriss et al., 2005). A lack of dietary β-carotene results in vitamin A deficiency, which is a leading cause of blindness and early mortality in people of third-world countries (Beyer et al., 2002). Lutein and zeaxanthin accumulate in the macular of the eye and are important in protecting the eye from damage and degeneration (Landrum and Bone, 2001). Carotenoids also act as anti-oxidants and assist in protection against free radical species that can damage cell DNA and lead to cancer. Indeed, consumption of fruits and vegetables high in carotene and lycopene may reduce the risk of cancer (Cramer et al., 2001).

### Carotenoid Accumulation in Higher Plants

Carotenoids accumulate in plastids, in particular in chloroplasts in photosynthetic tissues where they form part of the light harvesting complex with chlorophyll, in elaioplasts which are specialised lipid-storing plastids (Kirk and Tiliney-Bassett, 1978) and to some extent in amyloplasts which are the primary site of starch synthesis and storage. In coloured flowers and fruits they accumulate in specialised lipoprotein sequestering structures in chromoplasts (Bartley and Scolnik, 1995; Vishnevetsky et al., 1999). Lutein accounts for about 50% of the total carotenoid pool in chloroplasts, with the remainder being β-carotene, neoxanthin, violaxanthin and others (Pogson et al., 1996).

Two major mechanisms have been described that may regulate carotenoid biosynthesis and accumulation in plant tissues, namely altered transcription of regulatory or biosynthetic genes and altered or novel carotenoid accumulation due to the presence of sequestering structures capable of storing carotenoids within plastids. Phytoene synthase (PSY), the first committed step in the carotenoid pathway, has been identified as a key regulatory enzyme of carotenoid biosynthesis in tomato and other species (Giuliano et al., 1993; Welsch et al., 2000). In marigold flowers, the abundance of PSY mRNA transcript varies between varieties and correlates positively with carotenoid levels of the flowers (Moehs et al., 2001). Lycopene 8-cyclase is an enzyme that acts at the branch point of the carotenoid biosynthesis pathway, and is the first committed step in lutein biosynthesis (Pogson et al., 1996). Lutein is the major carotenoid in marigold petals and it has been proposed that ε-cyclase activity determines the level of lutein accumulation by increasing the degree to which lycopene is diverted into that branch of the pathway (Moehs et al., 2001).

Amyloplasts are ‘colourless’ plastids that are specialised for storage of starch granules (Kirk and Tiliney-Bassett, 1978) and hence characterise starchy seeds such as wheat and maize. Lutein is the major carotenoid present in the wheat amyloplasts (Hentschel et al., 2002) and seed amyloplasts of other plants also seem to accumulate mostly lutein. The wild type maize seed accumulates mostly lutein followed by zeaxanthin and a xanthophyll monoester as well as trace amounts of β-carotene (Janick-Buckner et al., 1999). White millet has 69% lutein, 29% zeaxanthin and trace (˜1%) β-carotene (McGraw et al., 2001). Lutein is also the major carotenoid present in oilseeds of many plants such as sunflower (McGraw et al., 2001), pumpkin (Matus et al., 1993) and canola (Shewmaker et al., 1999).

Esterification is a common means to sequester carotenoids in fruits and flowers. Carotenoid esters are formed by conventional esterification of the carotenoid hydroxyl group to fatty acids from acyl-CoA (Britton, 1998). Esterification does not affect the chromophore properties of the pigment and is postulated to increase xanthophyll accumulation by increase lipophilic properties and increasing integration into the lipid-rich plastoglobules (Hornero-Mendez and Minguez-Mosquera, 2000).

Seed carotenoids of various plants have been manipulated by both transgenic approaches and by insertional mutagenesis, and offer valuable insights into seed carotenogenesis. PSY has been over-expressed specifically in the seed of canola. Whereas wild-type canola seed contain lutein, transgenic plants that over-expressed PSY in seed had a 50-fold increase in total carotenoid content in the form of α and β-carotene (Shewmaker et al., 1999). This level of increase in carotenoids was dramatic compared to other transgenic studies (von Lintig et al., 1997) and was partially attributed to a change in morphology of the transgenic chromoplasts. It was noted that control of pigment accumulation in chloroplasts was very different to that in chromoplasts, elaioplasts or indeed other plastids where the carotenoids have quite different roles. This was exemplified in tobacco where overexpression of CrtO, a bacterial β-carotene ketolase gene under the control of a tomato PDS promoter, produced only trace amounts of astaxanthin in chloroplast-containing green tissue but a 170% increase in total carotenoids in the chromoplast-containing nectary tissue (Mann et al., 2000).

PSY has also been over-expressed in the endosperm of rice seed, which does not usually accumulate carotenoids, and transgenic seeds accumulated small amounts of phytoene (Burkhardt et al., 1997). Subsequently, PSY, PDS and β-cyclase were expressed in the endosperm of rice to produce seed accumulated β-carotene, lutein and zeaxanthin (Ye et al., 2000). This led to the development of ‘golden rice’ containing provitamin A (β-carotene) which could have positive impacts for people of third world countries suffering from vitamin A deficiencies and resulting blindness (Beyer et al., 2002).

The expression of PSY seems integral to the accumulation of carotenoids in both canola and rice. However, it is interesting that the accumulations favour the β-carotene branch of the pathway, and this may imply a rate-limiting enzyme on the α-carotene/lutein branch of the pathway. This was also seen when PSY was over-expressed in the seed of Arabidopsis to increase carotenoids, resulting mainly in an increased β-carotene content which rose to 28% of the total carotenoid pool (Lindgren et al., 2003).

Insertional mutagenesis resulting in altered levels of seed carotenoids, detected either visually or by HPLC, has been used to identify genes involved in the synthesis and accumulation of carotenoids in seed. Janick-Buckner et al. (1999) characterised the y10 mutant of maize, which exhibits a pale yellow kernel or seed. The wild type seed accumulates lutein, zeaxanthin and a xanthophyll monoester as well as trace amounts of β-carotene, whereas the y10 mutant had markedly reduced lutein and to a lesser extent, zeaxanthin (Janick-Buckner et al., 1999). The mutation was thought to affect a step prior to phytoene. The Arabidopsis lut1 mutant was characterised by significant decreases in lutein content of both leaf and seed tissue (Tian et al., 2003). The lut1 locus encoded ε-hydroxylase, which is a cytochrome P450-type monooxygenase (Tian et al., 2004) and which was required along with β-hydroxylase to catalyse the formation of lutein from α-carotene. This Arabidopsis cytochrome P450-type monooxygenase was the first genetically defined ε-hydroxylase and differed from the β-hydroxylase which was a non-heme diiron monooxygenase (Tian et al., 2004). Lutein accounts for 75% of the total carotenoid pool in wild-type Arabidopsis seed and this was reduced by 19% to 57% in lut1 mutant seed (Tian et al., 2003). Conversely, the β,β-xanthophylls increased to 42% of the total carotenoid pool in lut1 mutant seeds compared to about 24% in wild type seed, with much of this increase due to an increase in violaxanthin (Tian et al., 2003). This suggested that an increased flux into the β,β-xanthophyll pathway in seed resulted in an increased accumulation of later stage β-xanthophylls rather than their precursors. The 20% reduction in the lutein level of lut1 seeds was significantly less than 80% reduction of the lutein level in leaf tissue.

Much work regarding seed carotenogenesis has been done on maize, primarily because of the availability of the viviparous mutants which flag them as potential carotenoid mutants. As mentioned above, adequate β-carotenoids in seed are important for ABA production and seed dormancy. A Vp12 maize mutant that was deficient in carotenoids and ABA and had a viviparous phenotype was identified (Maluf et al., 1997). PDS has been associated with the viviparous 5 (vp5) mutant, a white endosperm mutant deficient in both carotenoids and ABA (Li et al., 1996). The yellow1 gene, of the viviparous y1 mutant with a white endosperm, has been linked to chromosome 6, which is associated with PSY (Buckner et al., 1996). Maize expresses products from duplicate PSY genes, PSY1 (Buckner et al., 1996), and PSY2 (Gallagher et al., 2004). Only PSY1 transcript abundance correlates with carotenoid content of maize endosperm (Buckner et al., 1996). Ortholog-specific PCR primers determined that the PSY duplication was present in 8 subfamilies of the Poaceae family including rice and wheat, Although PSY1 and PSY2 were both functional in bacterial systems, they are not functionally equivalent in planta and it seemed endosperm carotenoid accumulation required PSY1. The viviparous 9 (vp9) lesion, characterised as another white endosperm mutant of maize with zeta-carotene accumulation, had been mapped to chromosome 7S and was associated with zeta-carotene (ζ-carotene) desaturase (ZDS). Maize ZDS and PDS mediated a desaturation pathway which resulted predominately in prolycopene accumulation rather than all-trans-lycopene required by the downstream lycopene cyclase enzymes, β- and ε-cyclase (Matthews et al., 2003). All-trans-lycopene, the preferred substrate for the cyclases, was produced by the desaturases in concert with the carotenoid isomerase, CRTISO (Park et al., 2002). The activity of CRTISO in maize endosperm may also be a rate-controlling mechanism in seed carotenogenesis by affecting the availability all-trans-lycopene substrate.

Carotenoid content varies across a range of wheat germplasm. Durum varieties typically accumulate from 1.7 μg to 2.7 μg carotenoids/g flour, whereas bread wheat varieties ordinarily accumulate from about 0.40 μg to 1.9 μg/g. Hentschel et al., (2002) measured a variety of durum wheat cultivars and listed carotenoid contents between 2 and 4 μg carotenoid/g flour. Moss (1967) studied a range of Australian bread wheat germplasm and documented very high yellow pigment content for the cultivar Festival at 5.33 μg/g. Hordeum chilense, a wild barley species that can be crossed to wheat to generate new Triordeum lines, consistently show higher carotenoid pigment levels than wheat (Alvarez et al., 1994). When pigment content was measured in H. chilense x durum, a mean carotenoid content of 4 μg/g, with a range of 2.4 μg/g to 7.9 μg/g was documented (Alvarez et al., 1999).

There is clearly a genetic basis for carotenoid accumulation. Certain wheat cultivars produced flours with consistently higher carotenoid levels compared to other wheat cultivars, regardless of environmental conditions. A study performed on 67 white-seeded Australian accessions grown at two locations for 2 years showed genotype x location interactions were not significant for colorimetric and pigmentation variables (Matus-Cadiz et al., 2003) indicating the independent nature of genetic factors for grain colour. Pigment content in wheat flour was controlled by additive gene effects which were highly heritable, with heritability values ranging from 0.9 to 0.97 (Nachit et al., 1995). A subsequent study investigating genetic effects of carotenoid accumulation in durum wheat encompassed 16 environments and found heritability values >0.8 (Elouafi et al., 2001). QTLs (quantitative trait loci) for carotenoid accumulation in durum wheat were identified on chromosomes 2A and 2B (Joppa and Williams, 1988). More recently a major locus on chromosome 7B in durum wheat was reported that explained 53% of the carotenoid variation (Elouafi et al., 2001).

For bread wheat, a genetic region for flour colour has been identified in the cultivar Schomburgk on chromosome 7A. This QTL explained 60% of the variation for flour colour in the Schomburgk x Yarralinka mapping population (Parker et al., 1998). One molecular marker has been produced for the Schomburgk allele for the prediction of colour in breeding programs. This marker on chromosome 7A can be used to determine the presence or absence of an allele that contributes to the yellowness of flour (Parker & Landridge 2000, Sharp et al., 2001). However this marker was not suitable for use on all current Australian wheat varieties (Sharp et al., 2001). In addition to the Schomburgk x Yarralinka QTL on 7A, QTLs associated with flour colour have been identified on chromosome 3A (Schomburgh x Yarralinka) (Parker et al., 1998) as well as on chromosomes 3B and 7A (Sunco x Tasman) (Mares & Campbell, 2001).

Three carotenoid biosynthesis enzymes have been mapped in durum wheat (Cenci et al., 2004). PSY homeoforms were localised on chromosomes 5A and 5B, PDS on chromosomes 4A and 4B, and ZDS on chromosomes 2A and 2B (Cenci et al., 2004). The chromosome locations of PSY and ZDS map to QTLs identified for semolina colour (Blanco, unpublished data, see Cenci et al., 2004), but sequence polymorphisms between parental lines and fine mapping were required before associations between colour QTLs and carotenoid genes were conclusive.

There are limited numbers of QTLs that have been identified for flour colour in wheat and none of the key genes determining these traits have been identified. Therefore, there is a need for new wheat varieties to provide modified flour colour and methods to identify these.

### SUMMARY OF THE INVENTION

A large number of plant genes have been suggested to influence carotenoid biosynthesis. The present inventors have performed a complex analysis of the wheat genome in an attempt to identify genetic factors that play a role in the production lutein. This analysis resulted in the identification of at least one gene of the wheat genome that can be manipulated to alter the lutein content of, for example, wheat seeds.

In a first aspect, the present invention provides a substantially purified and/or recombinant polypeptide selected from:

i) a polypeptide comprising an amino acid sequence as provided in SEQ ID NO:22,

ii) a polypeptide comprising an amino acid sequence as provided in SEQ ID NO:23,

iii) a polypeptide comprising an amino acid sequence as provided in SEQ ID NO:24,

iv) a polypeptide comprising an amino acid sequence which is at least 88% identical to any one of i) to iii), and

v) a biologically active fragment of any one of i) to iv),

wherein the polypeptide has ε-cyclase activity.

Preferably, the polypeptide comprises an amino acid sequence which is at least 99% identical to SEQ ID NO:22, SEQ ID NO:23 or SEQ ID NO:24.

In one embodiment, when the polypeptide comprises an amino acid sequence which is less than 99% identical to SEQ ID NO:22, SEQ ID NO:23 or SEQ ID NO:24 it is preferred that the polypeptide is greater than 260 amino acids in length.

With regard to biologically active fragments, it is preferred that such fragments are greater than 30 amino acids in length.

Preferably, the polypeptide can be purified from wheat.

In another embodiment, the polypeptide is a fusion protein further comprising at least one other polypeptide sequence.

The at least one other polypeptide may be, for example, a polypeptide that enhances the stability of a polypeptide of the present invention, or a polypeptide that assists in the purification of the fusion protein.

Also provided is a substantially purified antibody, or fragment thereof, that specifically binds a polypeptide of the invention.

In an embodiment, the antibody is detectably labelled.

In another aspect, the present invention provides an isolated and/or exogenous polynucleotide comprising a sequence of nucleotides selected from:

i) a sequence of nucleotides as provided in SEQ ID NO:16,

ii) a sequence of nucleotides as provided in SEQ ID NO:17,

iii) a sequence of nucleotides as provided in SEQ ID NO:18,

iv) a sequence of nucleotides as provided in SEQ ID NO:19,

v) a sequence of nucleotides as provided in SEQ ID NO:20,

vi) a sequence of nucleotides as provided in SEQ ID NO:21,

vii) a sequence of nucleotides encoding a polypeptide of the invention,

viii) a sequence of nucleotides which is at least 86% identical to any one of i) to vi), and

ix) a sequence which hybridises to any one of i) to vi) under stringent conditions.

Preferably, the polynucleotide comprises a nucleotide sequence which is at least 98% identical to SEQ ID NO:19, SEQ ID NO:20 or SEQ ID NO:21.

In one embodiment, when the polynucleotide comprises a nucleotide sequence which is less than 99% identical to SEQ ID NO:19, SEQ ID NO:20 or SEQ ID NO:21 it is preferred that the polynucleotide is greater than 900 nucleotide in length.

In another embodiment, it is preferred that the polynucleotide is at least 150 nucleotides in length.

In a further aspect, the present invention provides an oligonucleotide which comprises at least 19 contiguous nucleotides of a polynucleotide of the invention.

Preferably, the oligonucleotide comprises at least 19 contiguous nucleotides of SEQ ID NO:19, SEQ ID NO:20 or SEQ ID NO:21.

In a further aspect, the present invention provides a polynucleotide which, when present in a cell of a cereal plant, down-regulates the level of ε-cyclase activity in the cell when compared to a cell that lacks said polynucleotide.

As discussed below, such polynucleotides can be produced in transgenic plants to reduce the level of lutein production in the seeds of said plants, thus altering the colour of flour, or a product obtained therefrom, which can be produced from the plants when compared to a wild-type plant.

Preferably, the polynucleotide is selected from, but not limited to, an antisense polynucleotide, a sense polynucleotide, a catalytic polynucleotide, a microRNA and a double stranded RNA.

In one embodiment, the polynucleotide is an antisense polynucleotide which hybridises under physiological conditions to a polynucleotide comprising a sequence of nucleotides as provided in SEQ ID NO:19, SEQ ID NO:20 or SEQ ID NO:21.

In another embodiment, the polynucleotide is a catalytic polynucleotide capable of cleaving a polynucleotide according to the invention. Examples of such catalytic polynucleotides include, but are not limited to, ribozyme and DNAzymes.

In a further embodiment, the polynucleotide is a double stranded RNA (dsRNA) molecule comprising an oligonucleotide of the invention, wherein the portion of the molecule that is double stranded is at least 19 basepairs in length and comprises said oligonucleotide.

Preferably, the dsRNA is expressed from a single promoter, wherein the strands of the double stranded portion are linked by a single stranded portion.

An example of a suitable dsRNA of the invention is encoded by a polynucleotide comprising a nucleotide sequence provided as SEQ ID NO:25.

In a further embodiment, a transgenic plant of this aspect of the invention has increased levels of violaxanthin in the seed of the plant when compared to a wild-type plant.

In a further aspect, the present invention provides a vector comprising or encoding a polynucleotide of the invention.

Preferably, the polynucleotide, or sequence encoding the polynucleotide, is operably linked to a promoter.

In another aspect, the present invention provides a host cell comprising a vector of the invention, and/or a polynucleotide of the invention.

The present inventors have determined that altering ε-cyclase activity in plants results in modified lutein content in the transgenic plants.

Thus, in a further aspect the present invention provides a transgenic plant,

wherein the transgenic plant has increased expression of a polypeptide having ε-cyclase activity relative to a corresponding non-transgenic plant.

Preferably, the plant has been transformed with a polynucleotide of the invention.

Preferably, the polynucleotide is capable of expression to produce a polypeptide of the invention.

In another embodiment, the expression level of at least one endogenous ε-cyclase gene has been increased relative to a corresponding non-transgenic plant. This can be achieved using techniques known in the art such as introducing highly active promoter elements upstream of the start codon of an ε-cyclase gene.

In another aspect, the present invention provides a transgenic plant, wherein the transgenic plant has decreased expression of a polypeptide having ε-cyclase activity relative to a corresponding non-transgenic plant. With regard to this aspect, it is preferred that ε-cyclase activity is only decreased in the seed, more preferably the endosperm of the seed, of the plant.

Preferably, the plant has been transformed such that it produces a polynucleotide of the invention which, when present in a cell of the plant, down-regulates the level of ε-cyclase activity in the cell when compared to a cell that lacks said polynucleotide.

In another embodiment, the transgenic plant has been transformed such that it produces a second polynucleotide which, when present in a cell of the plant, down-regulates the level of a polyphenol oxidase in the cell when compared to a cell that lacks said second polynucleotide. Alternatively, the plant may have at least one mutant polyphenol oxidase gene such that the level of polyphenol oxidase activity in a cell of a plant is reduced when compared to a wild-type plant.

Preferably, the transgenic plant is a cereal plant. More preferably, the cereal plant is a wheat or barley plant.

In yet a further aspect, the present invention provides a method of altering colour of flour produced from a cereal plant, the method comprising manipulating said plant such that the production of a polypeptide is modified when compared to a wild-type plant, wherein the polypeptide has ε-cyclase activity.

Preferably, the polypeptide is selected from:

i) a polypeptide comprising an amino acid sequence as provided in SEQ ID NO:22,

ii) a polypeptide comprising an amino acid sequence as provided in SEQ ID NO:23,

iii) a polypeptide comprising an amino acid sequence as provided in SEQ ID NO:24,

iv) a polypeptide comprising an amino acid sequence which is at least 88% identical to any one of i) to iii), and

v) a biologically active fragment of any one of i) to iv).

The present invention also provides methods of identifying alleles of ε-cyclase genes from cereal plants, such as wheat, that confer upon the plant desired levels of lutein in the endosperm of seeds of the plant, and especially alleles that confer upon the plant the ability to be used to produce flour, or a product obtained therefrom, with a desired colour.

Thus, in another aspect the present invention provides a method of genotyping a cereal plant, the method comprising detecting a nucleic acid of the plant or protein encoded thereby, wherein the nucleic acid molecule is genetically linked to, and/or comprises at least part of, a ε-cyclase gene.

In one embodiment, the method comprises determining the level of expression, and/or sequence, of a nucleic acid molecule of the plant encoding a polypeptide having ε-cyclase activity. Any suitable technique known in the art can be used such as, but not limited to, restriction fragment length polymorphism analysis, amplification fragment length polymorphism analysis, microsatellite amplification and/or nucleic acid sequencing.

In another embodiment, the method comprises determining the level of production, and/or activity, of a polypeptide having ε-cyclase activity. Again, any suitable technique known in the art can be used such as, but not limited to, detection of ε-cyclase protein levels using an antibody of the invention.

The markers of the invention can be used in cereal plant breeding programs to select progeny plants which possess an allele of an ε-cyclase gene of interest.

Accordingly, in a further aspect the present invention provides a method of selecting a cereal plant from a population of cereal plants, the method comprising;

i) genotyping said population of plants using a method of the invention, wherein said population of cereal plants was obtained from a cross between two plants of which at least one plant comprises an allele of an ε-cyclase gene which confers upon said plant the ability to be used to produce flour, or a product obtained therefrom, with a desired colour, and

ii) selecting said cereal plant on the basis of the presence or absence of said allele.

Preferably, the population is at least 10 plants, more preferably at least 20 to 50 plants.

Preferably, the cereal plant is a wheat or barley plant. More preferably, the plant is a wheat plant.

A further aspect provides a method of introducing an allele of an ε-cyclase gene which confers upon a cereal plant the ability to be used to produce flour, or a product obtained therefrom, with a desired colour into a cereal plant lacking said allele, the method comprising;

i) crossing a first parent cereal plant with a second parent cereal plant, wherein the second plant comprises an allele of an ε-cyclase gene which confers upon a cereal plant the ability to be used to produce flour, or a product obtained therefrom, with a desired colour, and

ii) backcrossing the progeny of the cross of step i) with plants of the same genotype as the first parent plant for a sufficient number of times to produce a plant with a majority of the genotype of the first parent but comprising said allele,

wherein progeny plants are genotyped for the presence or absence of said allele using a method of the invention.

Preferably, the cereal plant is a wheat or barley plant. More preferably, the plant is a wheat plant.

Preferably, the method further comprises analysing the plant for other genetic markers.

In another aspect, the present invention provides a cereal plant, or progeny thereof, produced using a method of the invention, or genotyped using a method of the invention.

Preferably, the cereal plant is a wheat or barley plant. More preferably, the plant is a wheat plant.

In one embodiment, a wheat plant of the invention is tetraploid wheat such as durum wheat.

In another embodiment, a wheat plant of the invention is hexaploid wheat.

In another embodiment, the hexaploid wheat lacks one functional ε-cyclase gene of the invention. In another embodiment, the hexaploid wheat lacks two functional ε-cyclase genes of the invention. Such plants can be produced using, for example, ecotilling as described herein. The ε-cyclase gene(s) can be from the A, B and/or D genome. In these instances, the plant would still produce polypeptides with ε-cyclase, just at a lower level when compared to a wild-type hexaploid wheat plant.

In another embodiment, the wheat plant has reduced levels of polyphenol oxidase activity in a cell of the plant when compared to a wild-type plant.

In a further embodiment, a wheat plant of the invention has increased levels of violaxanthin in the seed of the plant when compared to a wild-type plant.

In a further aspect, the present invention provides a method of producing seed, the method comprising;

a) growing a wheat and/or barley plant of the invention, and

b) harvesting the seed.

In another aspect, the present invention provides a seed of a wheat or barley plant of the invention.

In yet another aspect, the present invention provides a method of producing flour, wholemeal, or starch the method comprising;

a) obtaining seed of the invention, and

b) extracting the flour, wholemeal, or starch.

Also provided is a product produced from a seed of the invention and/or the plant of the invention. Such products can be food, for example food intended for consumption by mammals such as humans, or non-food products. Methods of producing such products are well known to those skilled in the art.

Examples of food products include, but are not limited to, flour, starch, leavened or unleavened breads, pasta, noodles, animal fodder, breakfast cereals, snack foods, cakes, pastries and foods containing flour-based sauces.

Examples of non-food products include, but are not limited to, films, coatings, adhesives, building materials and packaging materials.

The present inventors have provided methods of producing flour with a low content, or no, lutein and/or lutein esters. Such flour is particularly useful for producing bread and certain types of noodles. Thus, in a further aspect, the present invention provides flour which comprises less than about 0.40 μg/g of lutein and/or lutein ester.

Preferably, the lutein and/or lutein ester constitutes less than 0.30 μg per gram of the flour, more preferably less than 0.20 μg per gram of the flour, more preferably less than 0.10 μg per gram of the flour, more preferably less than 0.09 μg per gram of the flour, and more preferably less than 0.05 μg per gram of the flour.

In another aspect, the present invention provides flour which has a Minolta b* value of less than about 5. Preferably, the Minolta b* value is less than 4, more preferably less than 3, more preferably less than 2, and more preferably less than 1.

In a further aspect, the present invention provides grain which comprises less than about 25 μg of lutein and/or lutein ester per 100 g of grain. Preferably, the grain comprises less than 20 μg, more preferably less than 15 μg, more preferably less than 10 μg, more preferably less than 5 μg, and more preferably less than 1 μg of lutein and/or lutein ester per 100 g of grain.

Preferably, the grain is wheat grain or barley grain.

In preferred embodiment, the grain or flour is not from the wheat cultivar Pandas.

As noted above, also provided is a food or non-food product produced using flour and/or grain of the invention.

The present inventors have also found that reducing ε-cyclase activity and/or levels in a plant results in an increase in the production of violaxanthin.

Thus, in a further aspect, the present invention provides a method of increasing the levels of violaxanthin in a plant, the method comprising reducing the level and/or activity of at least one polypeptide with ε-cyclase activity in the plant.

Preferably, the polypeptide is a polypeptide of the invention.

Preferably, the plant is a wheat plant.

Also provided is a plant, preferably a wheat plant, produced using the above method.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Carotenoid biosynthetic pathway in higher plants. The pathway shows the primary steps found in nearly all plant species. The desaturases introduce a series of four double bonds in a cis-configuration, which are isomerized to the all-trans-conformations by the carotenoid isomerase. CRTISO appears to act in concert with the ζ-carotene desaturase, resulting in a complex mix of isomers with tetra-cis-lycopene the main product in the CRTISO mutant (ccr2). Additional Arabidopsis mutations are shown in italics. PSY, phytoene synthase; PDS, phytoene desaturase; ZDS, ζ-carotene desaturase; CRTISO, carotenoid isomerase; βLCY, β-cyclase; εLCY, β-cyclase; βOH, β-hydroxylase; εOH, ε-hydroxylase; ZE, zeaxanthin epoxidase; VDE, violaxanthin de-epoxidase; NXS, neoxanthin synthase; NCED, 9-cis-epoxycarotenoid

FIG. 2—Analysis of carotenoids in leaf, durum and bread wheat by HPLC. Absorbance (mAU) of column eluates were taken at 440 nm. Carotenoids and chlorophylls were detected in leaf tissue (upper panel) and lutein was detected in flour samples (mid and lower panels), along with some unidentified carotenoids in bread wheat flour (lower panel). Scale differences are due to different amounts of tissue extracted.

FIG. 3—Analysis of carotenoids in bread wheat by HPLC before (upper panel) and after (lower panel) saponification, demonstrating the presence of lutein esters.

FIG. 4—HPLC analysis of carotenoids in wheat grain endosperm (10 DPA, lower panel) compared to leaf (upper panel). Absorbance scales differ due to different amounts of tissue used as well as different carotenoid contents of tissues. The leaf carotenoid profile showed the standard retention times of the carotenoids neoxanthin, violaxanthin, lutein and β-carotene, as well as chlorophyll a and chlorophyll b. The endosperm carotenoid profile at 10 DPA showed the presence of violaxanthin, antheraxanthin, lutein and zeaxanthin.

FIG. 5—Carotenoid profiles during endosperm development at 10 (panel a), 15 (panel b), 20 (panel c), 30 (panel d) and 40 DPA (panel e). Different amounts of endosperm tissue were used for each of the time points, therefore net and relative carotenoid contents could not be quantified accurately.

FIG. 6—Distribution of lutein levels (in μg/g flour, including lutein esters) in flour from lines in a Sunco x Tasman doubled haploid population. The frequency of wheat lines within each class (0.5 μg increments) was graphed against the amount of lutein. Lutein content for the population followed a normal distribution pattern as shown by the continuous trend line.

FIG. 7—Distribution of lutein ester content as a percentage of total lutein in the Sunco x Tasman doubled haploid population. The number of wheat lines was graphed against the percentage of lutein esters in flour.

FIG. 8—The QTL for lutein content on 3B. Each marker interval on chromosome 3B was tested for the likelihood that the chromosome segment was associated with lutein accumulation in wheat endosperm. The segments of closest association centred on marker gwn285 and were significantly associated with lutein accumulation in wheat endosperm (LOD 13.4).

FIG. 9—The QTLs for lutein content on 5B. Each marker interval on chromosome 5B was tested for association with lutein accumulation in wheat endosperm. The interval centering on marker wmc376 was most strongly associated with lutein accumulation (LOD 15.1). Additional intervals near markers gwm067, P46/M373 and P32/M48-234 as well as near markers P36/M401 had similar LOD scores and indicate additional lutein QTLs on 5B. Potentially, there are 3 distinct QTLs for lutein content on 5B, labelled 1, 2 and 3 in order of significance in the figure above. The QTL on 5B previously detected for b* values of flour was most likely the QTL labelled no. 3 in the figure above.

FIG. 10—The QTL for lutein content on 7A. Each marker interval on chromosome 7A was tested for the likelihood of association with lutein accumulation in wheat endosperm. The QTL identified on 7A was strongly associated with the region near markers P41/M55-199 and P32/M52-181, as circled in the figure above (LOD 22.1). The four markers below this segment (lower box) also show significant association, but are most likely part of the same QTL. The QTL detected on 7A by other studies also covers the region of all these markers.

FIG. 11—Comparison of three partial wheat endosperm cDNA sequences with corresponding rice PSY cDNA sequence. The nucleotide numbering is relative to the rice cDNA (Accession No. AY024351).

FIG. 12—Alignment of genomic DNA sequence fragments from 3 PSY homeoforms from wheat. The sequence was amplified from Chinese Spring DNA using the homeoform specific primers (Table 4) and show 12 SNP differences and a deletion in the intron of PSY2 (dotted).

FIG. 13—Comparison of wheat ε-cyclase cDNA sequences, expressed in endosperm, with rice ε-cyclase cDNA sequence. The nucleotide numbering is relative to the rice cDNA (AP003332). SNPs identified between the wheat cDNA sequences (cv. Chinese Spring) and nucleotide differences with the rice sequence are shown in lower case.

FIG. 14—Comparison of wheat genomic ε-cyclase DNA sequences with rice ε-cyclase genomic sequence including the position of the intron. SNPs identified within wheat gDNA sequences (cv. Chinese Spring) are highlighted. Also highlighted are the two reverse primers specific for homeoform 1 or 2 (green).

FIG. 15—Detection of wheat ε-cyclase genes by using homeoform-specific primer pairs and DNA from nullisomic/tetrasomic or diteleo lines in a Chinese Spring background. Lack of a PCR product in a particular lane showed that the gene homeoform was absent from the gDNA and hence that homeoform was located on the chromosome missing from that nulli/tetra line. ε-cyclase homeoform 1 was thus located on chromosome 3D, homeoform 2 on 3A and homeoform 3 on 3B. Lane 1: 50 bp ladder, Lane 2: No DNA control (ecycform1 primers), Lane 3: Chinese Spring gDNA (ecycform1 primers), Lane 4: null 3A gDNA (ecycform1 primers), Lane 5: null 3B gDNA (ecycform1 primers), Lane 6: null 3D gDNA (ecycform1 primers), Lane 7: No DNA control (ecycform2 primers), Lane 8: Chinese Spring gDNA (ecycform2 primers), Lane 9: null 3A gDNA (ecycform2 primers), Lane 10: null 3B gDNA (ecycform2 primers), Lane 11: null 3D gDNA (ecycform2 primers), Lane 12: 50 bp ladder, Lane 13: No DNA control (ecycform3 primers), Lane 14: null 3A gDNA (ecycform3 primers), Lane 15: null 3B gDNA (ecycform3 primers), Lane 16: null 3D gDNA (ecycform3 primers), Lane 17: null 3BL gDNA (ecycform3 primers), Lane 18: No DNA control (ecycform1 primers), Lane 19: null 3A gDNA (ecycform1 primers), Lane 20: null 3B gDNA (ecycform1 primers); Lane 21: null 3D gDNA (ecycform1 primers), Lane 22: null 3DS gDNA (ecycform1 primers), and Lane 23: null 3DL gDNA (ecycform1 primers).

FIG. 16—Exon/Intron structure of ε-cyclase from a) rice and b) wheat. Exons appear as boxes and introns appear as lines. Each exon and intron is numbered in rice. Rice and wheat have a conserved exon/intron structure, including the first large intron containing a transposon. Sequence 5′ of the first intron of wheat has not been obtained, but based on synteny with rice, it is predicted one more exon is required to complete the full-length gDNA from wheat. NB. Figure to scale.

FIG. 17—pStarling/ε-cyclase RNAi construct. The pStarling vector contained the ubiquitin promoter. The plasmid contained the ampicillin resistance gene for bacterial selection. Two insertions of the ε-cyclase fragment were cloned, one in forward and the other in reverse orientation on either side of the cre intron. The plantrnaif and plantrnair primers used to amplify the ε-cyclase fragment contained the restriction sites required for cloning. Kpn I and Spe I sites were used for cloning the first ε-cyclase fragment in the reverse orientation. The restriction sites Xma I and Asc I were used to clone the second ε-cyclase fragment in the forward orientation.

FIG. 18—pBx17/ε-cyclase RNAi construct. The pBx17 vector contained the Bx17 promoter for selective tissue expression of the inhibitory RNA in endosperm. The plasmid contained the kanamycin resistance gene for bacterial selection. Two inserts of the ε-cyclase fragment, one in forward and the other in reverse orientation were inserted on either side of the Rint9 intron into the pBx17 vector to produce the Rint9/e-cyclase construct.

FIG. 19—Altered endosperm colour in seeds of some lines containing a construct to silence ε-cyclase in the endosperm.

FIG. 20—Relative lutein content of flour from transgenic wheat lines containing a seed-specific construct to decrease expression of wheat epsilon cyclase genes. The peak area of the lutein peak for flour samples from individual plants was calculated and converted to a value per gram of flour, this was then divided by the corresponding value from the control plants. A value of 1 indicates that the lutein content was the same as that in the control, while a value lower than one is indicative of a decrease in lutein content of the flour compared to the control. The control plants were generated from embryos that had been shot with gold particles, which had not been coated with DNA, and regenerated through tissue culture in the same manner as the transgenic lines.

FIG. 21—Relative percentage of each carotenoid in leaves from T1 wheat plants containing an dsRNA construct to decrease expression of epsilon cyclase in the whole plant. The data presented for each line are the average from at least 8 independent plants, except for lines 11e1-2.1-3 and 11e1-2.1-9 which are individual progeny plants from line 11e1-2.1 in which the relative lutein content was decreased when compared to the other plants within the line. The control plants were generated from embryos that had been shot with gold particles, which had not been coated with DNA, and regenerated through tissue culture in the same manner as the transgenic lines.

FIG. 22—Lutein to violaxanthin ratio in leaves from T1 wheat plants based on the data as for FIG. 21.

FIG. 23—Lutein to chlorophyll b ratio in leaves from T1 wheat plants based on the data as for FIG. 21.

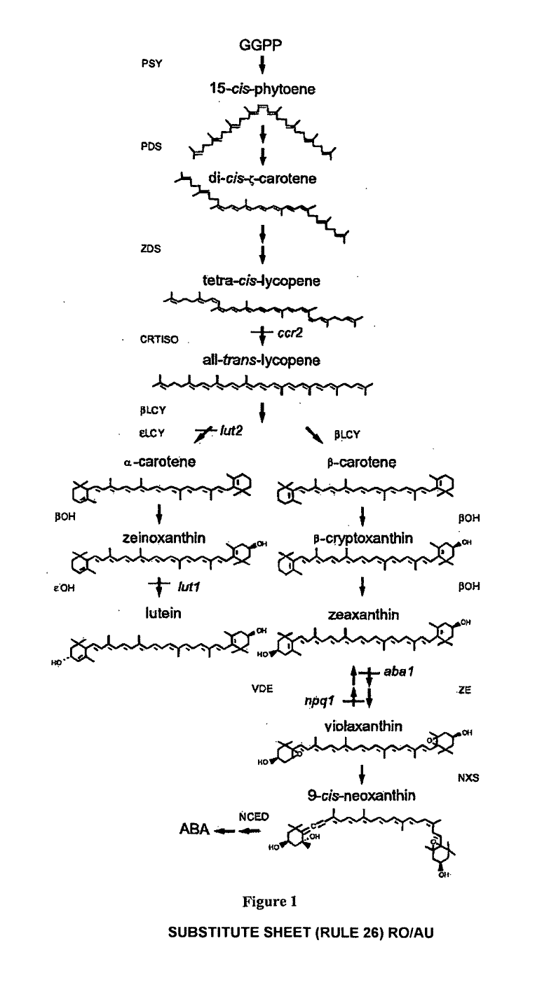

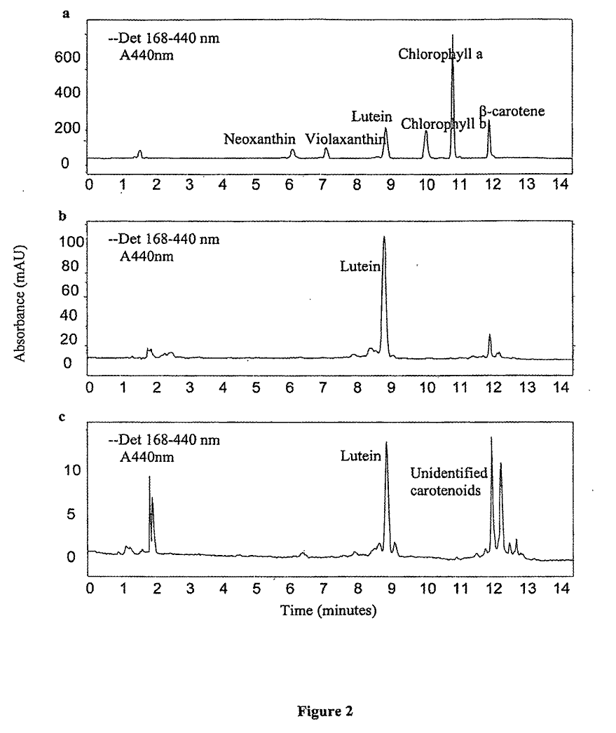

### KEY TO THE SEQUENCE LISTING

SEQ ID NO:1—Partial coding sequence of isoform 2 of wheat PSY.

SEQ ID NO:2—Partial coding sequence of isoform 3 of wheat PSY.

SEQ ID NO:3—Partial coding sequence of isoform 1 of wheat PSY.

SEQ ID NO:4—Partial coding sequence of a rice PSY (Accession No. AY024351).

SEQ ID NO:5—Partial sequence of wheat PSY isoform 1 gene.

SEQ ID NO:6—Partial sequence of wheat PSY isoform 3 gene.

SEQ ID NO:7—Partial sequence of wheat PSY isoform 2 gene.

SEQ ID NO:8—Partial coding sequence of isoform 1 of wheat ε-cyclase.

SEQ ID NO:9—Partial coding sequence of isoform 3 of wheat ε-cyclase.

SEQ ID NO:10—Partial coding sequence of isoform 2 of wheat ε-cyclase.

SEQ ID NO:11—Partial coding sequence of a rice ε-cyclase (Accession No. AP003332).

SEQ ID NO:12—Partial sequence of wheat ε-cyclase isoform 3 gene.

SEQ ID NO:13—Partial sequence of wheat ε-cyclase isoform 1 gene.

SEQ ID NO:14—Partial sequence of wheat ε-cyclase isoform 2 gene.

SEQ ID NO:15—Partial sequence of a rice ε-cyclase gene.

SEQ ID NO:16—Genomic sequence of the A genome homeoform of ε-cyclase from wheat cv. Chinese Spring.

SEQ ID NO:17—Genomic sequence of the D genome homeoform of ε-cyclase from wheat cv. Chinese Spring.

SEQ ID NO:18—Genomic sequence of the B genome homeoform of ε-cyclase from wheat cv. Chinese Spring.

SEQ ID NO:19—Coding sequence of the A genome homeoform of ε-cyclase from wheat cv. Chinese Spring.

SEQ ID NO:20—Coding sequence of the D genome homeoform of ε-cyclase from wheat cv. Chinese Spring.

SEQ ID NO:21—Coding sequence of the B genome homeoform of ε-cyclase from wheat cv. Chinese Spring.

SEQ ID NO:22—Amino acid sequence of the A genome isoform of ε-cyclase from wheat cv. Chinese Spring.

SEQ ID NO:23—Amino acid sequence of the D genome isoform of ε-cyclase from wheat cv. Chinese Spring.

SEQ ID NO:24—Amino acid sequence of the B genome isoform of ε-cyclase from wheat cv. Chinese Spring.

SEQ ID NO:25—Construct for use in silencing wheat ε-cyclase genes.

SEQ ID NO's 26 to 30—Sense strands of siRNA. Reverse complement of sequences provided comprise said siRNA.

SEQ ID NO's 31 to 69—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, wheat breeding, transgenic plants, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Selected Definitions

As used herein, a polypeptide with “ε-cyclase activity” is able to use lycopene (preferably all-trans-lycopene) as a substrate to produce δ-carotene which in turn may then be converted by β-cyclase to α-carotene.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. Tetraploid wheat includes T. durum (also referred to herein as durun wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare.

As used herein, the term “genetically linked” or similar refers to a marker locus and a second locus being sufficiently close on a chromosome that they will be inherited together in more than 50% of meioses, e.g., not randomly. This definition includes the situation where the marker locus and second locus form part of the same gene. Furthermore, this definition includes the embodiment where the marker locus comprises a polymorphism that is responsible for the trait of interest (in other words the marker locus is directly “linked” or “perfectly linked” to the phenotype). In another embodiment, the marker locus and a second locus are different, yet sufficiently close on a chromosome that they will be inherited together in more than 50% of meioses. The percent of recombination observed between genetically linked loci per generation (centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM apart and most preferably about 0 cM apart.

As used herein, the “other genetic markers” may be any molecules which are linked to a desired trait of a cereal plant such as wheat. Such markers are well known to those skilled in the art and include molecular markers linked to genes determining traits such disease resistance, yield, plant morphology, grain quality, flour colour and the like. Examples of such genes are stem-rust resistance genes Sr2 or Sr38, the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance (Eagles et al., 2001; Langridge et al., 2001; Sharp et al., 2001).

As used herein, the term “polyphenol oxidase” (PPO) refers to an enzyme that catalyses the oxidation of phenolic compounds, which can result in a darkening of flour samples after milling and exposure to oxygen. There are two groups of PPO genes in wheat. One family is represented by the sequences with accession numbers AY596268, AY596269 and AY596270, AY515506, while the second is represented by the sequences AF507945, AY596266 and AY596267 (Jukanti et al., 2004). A STS marker (PPO18) that discriminates between cultivars with high and low PPO levels in the grain has been developed. The primers for PPO18 were designed from the AY596268 sequence, and this has been mapped to the long arm of chromosome 2, between the markers Xgwm312 and Xgwm29. QTL analysis revealed a major QTL for PPO activity that co-segregated with PPO18 and accounted for 28-43% of the phenotypic variance across three environments (Sun et al., 2005).

The term “plant” includes whole plants, vegetative structures (for example, leaves, stems), roots, floral organs/structures, seed (including embryo, endosperm, and seed coat), plant tissue (for example, vascular tissue, ground tissue, and the like), cells and progeny of the same.

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes.

As used herein, the term “corresponding non-transgenic plant” refers to a wild-type plant. “Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein. With regard to wheat plants, transgenic wheat plants of the invention can be compared to wild type plant cultivars such as Sunco and Tasman.

The terms “seed” and “grain” are used interchangeably herein. “Grain” generally refers to mature, harvested grain but can also refer to grain after imbibition or germination, according to the context. Mature grain commonly has a moisture content of less than about 18-20%.

“Nucleic acid molecule” refers to a oligonucleotide, polynucleotide or any fragment thereof. It may be DNA or RNA of genomic or synthetic origin, double-stranded or single-stranded, and combined with carbohydrate, lipids, protein, or other materials to perform a particular activity defined herein.

As used herein, the term “nucleic acid amplification” refers to any in vitro method for increasing the number of copies of a nucleic acid molecule with the use of a DNA polymerase. Nucleic acid amplification results in the incorporation of nucleotides into a DNA molecule or primer thereby forming a new DNA molecule complementary to a DNA template. The newly formed DNA molecule can be used a template to synthesize additional DNA molecules.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate cell (preferably a wheat cell). Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of at least about 2 kb on either end. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

An “allele” refers to one specific form of a genetic sequence (such as a gene) within a cell, an individual plant or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

### Colour Determination of Flour

Cereal plants of the invention, transgenic or non-transgenic, such as wheat and barley can be used to produce flour with desired colour characteristics. Generally, it is preferred that flour for use in the production of bread and noodles contains low amounts of (or no) lutein resulting in white flour, whereas flour for use in the production of pasta contains high amounts of lutein resulting in yellow flour.

The routine method for defining the brightness and whiteness of flour samples uses the CIE (Commision International de l'Eclairage) measurement system. CIE is an international organisation which develops world standard measurement systems in the field of light and colour (http://www.cie.co.at). In the CIE system, a Minolta instrument measures the sample reflectance at three wavelength ranges to determine L*, a* and b* values. All colours that can be perceived visually can be measured with the L*, a*, b* scale (Good, 2002) but for flour colour analysis only the L* and b* values are useful. L* values indicate the brightness of the flour and b* values indicate the whiteness/creaminess of flours (Feillet et al., 2000; Good, 2002). b* is calculated on reflectance of the flour sample in the 400-500 nm region of the visible spectrum where yellow coloured compounds absorb light and so additionally can be described as a measure of yellowness and is often attributed to xanthophyll content (Mares et al., 1997). Lower CIE b* values indicate a whiter/creamier flour hence the requirement for bread and noodle wheats is a low b* value. Conversely, the requirement for durum wheat and pasta products is a high b* value, which indicates the yellow pigments are present at desired high levels (Mares and Campbell, 2001; Good, 2002).

The CIE measurement system for flour colour analysis is influenced by bran content and particle size, which is related to the milling and sieving processes (Mares and Campbell, 2001), hence a more accurate analysis of flour colour is spectrophometric measurement of the pigment after the pigment is extracted from flour. The standard AACC (American Association of Cereal Chemists) protocol for this type of pigment analysis uses water-saturated butanol for flour extraction. To determine yellow pigment content, absorbance of the extract is taken at 440 nm, which is then compared to β-carotene standards, and expressed as carotene content (ppm or μg/g) (AACC, 2000; Santra et al., 2003).

However, carotenoids determining flour colour are most accurately quantified and identified by HPLC. For HPLC, pigments are extracted from flour using a choice of organic solvents. The ranges of organic solvents that have been used to extract pigments from wheat include water-saturated butanol (Kaneko et al., 1995) hexane/diethyl ether (Ward et al., 1997) acetone/petroleum ether (Ward et al., 1996) and methanol/tetrahydrofuran (Hentschel et al., 2002). Pigment extracts are run on HPLC under an increasingly polar solvent and pigments are separated according to their hydrophobicity. The more hydrophobic the compound is, the longer the retention time will be. Traces obtained from HPLC analysis show peaks at different retention times, and the absorbance spectra of each peak can be measured. Each component can be identified with reference to standards with known spectra and retention times (Pogson et al., 1996).

In a preferred embodiment, lutein content of flour of the invention is determined using the method of Examples 1 and 2 provided herein.

### Polypeptides

By “substantially purified polypeptide” we mean a polypeptide that has generally been separated from the lipids, nucleic acids, other peptides, and other contaminating molecules with which it is associated in its native state. Preferably, the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length.

As used herein a “biologically active” fragment is a portion of a polypeptide of the invention which maintains a defined activity of the full-length polypeptide, namely is able to convert lycopene to 8-carotene. Biologically active fragments can be any size as long as they maintain the defined activity.

With regard to a defined polypeptide/enzyme, it will be appreciated that % identity figures higher than those provided above will encompass preferred embodiments. Thus, where applicable, in light of the minimum % identity figures, it is preferred that the polypeptide/enzyme comprises an amino acid sequence which is at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes related to those of the present invention, such as ε-cyclase genes from plant species other than wheat. Products derived from mutated/altered DNA can readily be screened using techniques described herein to determine if they possess ε-cyclase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

### Polynucleotides and Oligonucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 88%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In an embodiment, a polynucleotide of the invention is not that provided as Genbank Accession No. BQ240373.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Oligonucleotides and/or nucleic acids of the invention hybridize to a cereal plant ε-cyclase gene or PSY gene, or a region of the genome of said plant genetically linked thereto, under stringent conditions. The term “stringent hybridization conditions” and the like as used herein refers to parameters with which the art is familiar, including the variation of the hybridization temperature with length of an oligonucleotide. Nucleic acid hybridization parameters may be found in references which compile such methods, Sambrook, et al. (supra), and Ausubel, et al. (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). Alternatively, the nucleic acid and/or oligonucleotides (which may also be referred to as “primers” or “probes”) hybridize to the region of the wheat plant genome of interest under conditions used in nucleic acid amplification techniques such as PCR.

Oligonucleotides of the present invention can be RNA, DNA, or derivatives of either. Although the terms polynucleotide and oligonucleotide have overlapping meaning, oligonucleotide are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

The present invention includes oligonucleotides that can be used as, for example, probes to identify nucleic acid molecules, or primers to produce nucleic acid molecules. Oligonucleotide of the present invention used as a probe are typically conjugated with a detectable label such as a radioisotope, an enzyme, biotin, a fluorescent molecule or a chemiluminescent molecule.

Oligonucleotides of the invention are useful in methods of detecting an allele of an ε-cyclase gene or PSY gene linked to a flour colour trait of interest. Such methods, for example, employ nucleic acid hybridization and in many instances include oligonucleotide primer extension by a suitable polymerase (as used in PCR).

A variant of an oligonucleotide of the invention includes molecules of varying sizes of, and/or are capable of hybridising, for example, to the wheat genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides) to the region of the plant genome where the specific oligonucleotides defined herein hybridise.

### Antisense Polynucleotides

The term “antisense polynucleotide” shall be taken to mean a DNA or RNA, or combination thereof, molecule that is complementary to at least a portion of a specific mRNA molecule encoding a polypeptide of the invention and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque (1995) and Senior (1998). Bourque (1995) lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

As used herein, the term “an antisense polynucleotide which hybridises under physiological conditions” means that the polynucleotide (which is fully or partially single stranded) is at least capable of forming a double stranded polynucleotide with mRNA encoding a protein, such as a protein comprising an amino acid sequence provided in SEQ ID NO:22, SEQ ID NO:23 or SEQ ID NO:24, under normal conditions in a cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition. The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988, Perriman et al., 1992) and the hairpin ribozyme (Shippy et al., 1999).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for the ribozymes of this invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides of the invention should also be capable of hybridizing a target nucleic acid molecule (for example an mRNA encoding a polypeptide provided as SEQ ID NO:22, SEQ ID NO:23 or SEQ ID NO:24) under “physiological conditions”, namely those conditions within a cell (especially conditions in a wheat plant cell).

### RNA Interference

RNA interference (RNAi) is particularly useful for specifically inhibiting the production of a particular protein. Although not wishing to be limited by theory, Waterhouse et al., (1998) have provided a model for the mechanism by which dsRNA can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al., (1998), Smith et al., (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous plant system that destroys both the double stranded RNA and also the homologous RNA transcript from the target plant gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-21 contiguous nucleotides of the target mRNA. Preferably, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the plant (preferably wheat) in which it is to be introduced, e.g., as determined by standard BLAST search. Examples of siRNA molecules that may be used to down-regulate the production of a polypeptide with ε-cyclase activity comprise a sequence selected from, but not limited to, the nucleotide sequences provided in SEQ ID NOs: 26 to 30.

An example of a construct useful for RNAi is provided as SEQ ID NO:25.

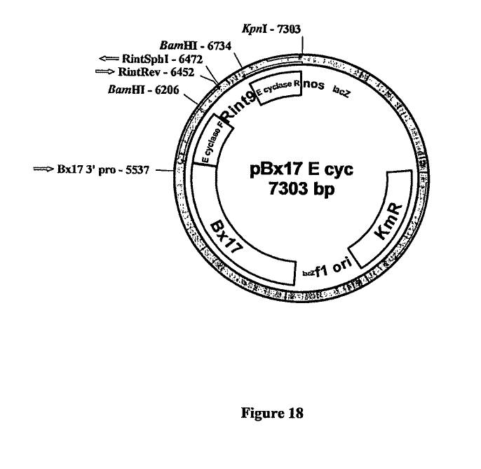

An example of a vector useful for the expression of RNAi constructs is provided in FIG. 18.

### MicroRNA

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Cosuppression

Another molecular biological approach that may be used is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target plant gene. Reference is made to WO 97/20936 and EP 0465572 for methods of implementing co-suppression approaches.

### Nucleic Acid Constructs, Vectors and Host Cells

The present invention includes the production of various transgenic plants. These include, but are not limited to, i) plants that express a polynucleotide of the invention which encodes a polypeptide having ε-cyclase activity, ii) plants where the expression level of at least one endogenous ε-cyclase gene has been increased relative to a corresponding non-transgenic plant, and iii) plants that express a polynucleotide which, when present in a cell of a cereal plant, down-regulates the level of ε-cyclase activity in the cell when compared to a cell that lacks said polynucleotide.

Nucleic acid constructs useful for producing the above-mentioned transgenic plants can readily be produced using standard techniques.

When inserting a region encoding an mRNA the construct may comprise intron sequences. These intron sequences may aid expression of the transgene in the plant. The term “intron” is used in its normal sense as meaning a genetic segment that is transcribed but does not encode protein and which is spliced out of an RNA before translation. Introns may be incorporated in a 5′-UTR or a coding region if the transgene encodes a translated product, or anywhere in the transcribed region if it does not. However, in a preferred embodiment, any polypeptide encoding region is provided as a single open reading frame. As the skilled addressee would be aware, such open reading frames can be obtained by reverse transcribing mRNA encoding the polypeptide.

To ensure appropriate expression of the gene encoding an mRNA of interest, the nucleic acid construct typically comprises one or more regulatory elements such as promoters, enhancers, as well as transcription termination or polyadenylation sequences. Such elements are well known in the art.

The transcriptional initiation region comprising the regulatory element(s) may provide for regulated or constitutive expression in the plant. Preferably, expression at least occurs in cells of the seed and/or developing seed. The regulatory elements may be selected be from, for example, seed-specific promoters, or promoters not specific for seed cells (such as ubiquitin promoter or CaMV35S or enhanced 35S promoters).

Examples of seed specific promoters useful for the present invention include, but are not limited to, the wheat low molecular weight glutenin promoter (Colot et al., 1987), the promoter expressing α-amylase in wheat seeds (Stefanov et al., 1991), and the hordein promoter (Brandt et al., 1985).

The promoter may be modulated by factors such as temperature, light or stress. Ordinarily, the regulatory elements will be provided 5′ of the genetic sequence to be expressed. The construct may also contain other elements that enhance transcription such as the nos 3′ or the ocs 3′ polyadenylation regions or transcription terminators.

Typically, the nucleic acid construct comprises a selectable marker. Selectable markers aid in the identification and screening of plants or cells that have been transformed with the exogenous nucleic acid molecule. The selectable marker gene may provide antibiotic or herbicide resistance to the wheat cells, or allow the utilization of substrates such as mannose. The selectable marker preferably confers hygromycin resistance to the wheat cells.

Preferably, the nucleic acid construct is stably incorporated into the genome of the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one isolated polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a cereal plant, more preferably barley or wheat cells, and even more preferably a wheat cell.

### Transgenic Plants

### General

As noted above, the term “plant” as used herein as a noun refers to whole plants, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plants provided by or contemplated for use in the practice of the present invention include both monocotyledons and dicotyledons. In preferred embodiments, the plants of the present invention are crop plants (for example, cereals and pulses, maize, wheat, potatoes, tapioca, rice, sorghum, millet, cassaya, barley, or pea), or other legumes. The plants may be grown for production of edible roots, tubers, leaves, stems, flowers or fruit.

Preferably, the transgenic plant is a cereal plant. Examples of cereal plants include, but are not limited to, wheat, barley, oats, and rye. More preferably, the cereal plant is wheat or barley.

Transgenic plants, as defined in the context of the present invention include plants and their progeny which have been genetically modified using recombinant techniques. This would generally be to modulate the production of at least one protein/enzyme defined herein in the desired plant or plant organ. Transgenic plant parts include all parts and cells of said plants such as, for example, cultured tissues, callus and protoplasts. Transformed plants contain genetic material that they did not contain prior to the transformation. The genetic material is preferably stably integrated into the genome of the plant. The introduced genetic material may comprise sequences that naturally occur in the same species but in a rearranged order or in a different arrangement of elements, for example an antisense sequence. Such plants are included herein in “transgenic plants”. A “non-transgenic plant” is one which has not been genetically modified with the introduction of genetic material by recombinant DNA techniques. In a preferred embodiment, the transgenic plants are homozygous for each and every gene that has been introduced (transgene) so that their progeny do not segregate for the desired phenotype.

Several techniques exist for introducing foreign genetic material into a plant cell. Such techniques include acceleration of genetic material coated onto microparticles directly into cells (see, for example, U.S. Pat. No. 4,945,050 and U.S. Pat. No. 5,141,131). Plants may be transformed using Agrobacterium technology (see, for example, U.S. Pat. No. 5,177,010, U.S. Pat. No. 5,104,310, U.S. Pat. No. 5,004,863, U.S. Pat. No. 5,159,135). Electroporation technology has also been used to transform plants (see, for example, WO 87/06614, U.S. Pat. Nos. 5,472,869, 5,384,253, WO 92/09696 and WO 93/21335). In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during development and/or differentiation using appropriate techniques described herein.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Any of several methods may be employed to determine the presence of a transformed plant. For example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers that will distinguish the transformed and non-transformed plants. For example, primers may be designed that will amplify a region of DNA from the transformation vector reading into the construct and the reverse primer designed from the gene of interest. These primers will only amplify a fragment if the plant has been successfully transformed. An alternative method to confirm a positive transformant is by Southern blot hybridization, well known in the art. Plants which are transformed may also be identified i.e. distinguished from non-transformed or wild-type plants by their phenotype, for example conferred by the presence of a selectable marker gene, or conferred by the phenotype of a desired colour of flour produced from the seed of the plant.

It is preferred that a seed of a transgenic plant of the invention has a germination rate which is substantially the same as that of the isogenic non-transgenic seed.

As used herein, “germination” refers to the emergence of the root tip from the seed coat after imbibition. “Germination rate” refers to the percentage of seeds in a population which have germinated over a period of time, for example 7 or 10 days, after imbibition. A population of seeds can be assessed daily over several days to determine the germination percentage over time.

With regard to seeds of the present invention, as used herein the term “germination rate which is substantially the same” means that the germination rate of the transgenic seeds is at least 60%, more preferably at least 80%, and even more preferably at least 90%, that of isogenic non-transgenic seeds. Germination rates can be calculated using techniques known in the art.

It is further preferred that the timing of germination of a seed of a transgenic plant of the invention is substantially the same as that of the isogenic non-transgenic seed.

With further regard to seeds of the present invention, as used herein the term “timing of germination of the seed is substantially the same” means that the timing of germination of the transgenic seeds is at least 60%, more preferably at least 80%, and even more preferably at least 90%, that of isogenic non-transgenic seeds. Timing of germination can be calculated using techniques known in the art.

### Transformation Methods for Cereal Plants

Methods for transformation of cereal plants such as wheat and barley for introducing genetic variation into the plant by introduction of an exogenous nucleic acid and for regeneration of plants from protoplasts or immature plant embryos are well known in the art, see for example, Wan and Lemaux (1994), Tingay et al., (1997), Canadian Patent Application No. 2,092,588, Australian Patent Application No 61781/94, Australian Patent No 667939, U.S. Pat. No. 6,100,447, International Patent Application PCT/US97/10621, U.S. Pat. No. 5,589,617, U.S. Pat. No. 6,541,257, and other methods are set out in Patent specification WO99/14314. Preferably, transgenic wheat plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

### Marker Assisted Selection

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis of at least one ε-cyclase gene, allows rapid selection of plants carrying the desired trait, which may be nurtured to maturity in the greenhouse or field for subsequent further backcrossing to the recurrent parent.

Any molecular biological technique known in the art which is capable of detecting alleles of an ε-cyclase gene or PSY gene can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of an ε-cyclase gene or PSY gene which confer to the seeds of the plant the ability to be used to produce flour of a desired colour and/or seeds with a desired lutein content. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al., (2001).

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells expressing an ε-cyclase gene or PSY gene. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al., (supra). Sequencing can be carried out by any suitable method, for example, dideoxy sequencing, chemical sequencing or variations thereof. Direct sequencing has the advantage of determining variation in any base pair of a particular sequence.

Hybridization based detection systems include, but are not limited to, the TaqMan assay and molecular beacons. The TaqMan assay (U.S. Pat. No. 5,962,233) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacon assay (U.S. Pat. No. 5,925,517). In the molecular beacon assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

### Tilling

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Antibodies

The invention also provides monoclonal or polyclonal antibodies to polypeptides of the invention or fragments thereof. Thus, the present invention further provides a process for the production of monoclonal or polyclonal antibodies to polypeptides of the invention.

The term “binds specifically” refers to the ability of the antibody to bind to proteins of the present invention but not other known ε-cyclases such as the protein encoded by Genbank Accession No. AP003332 (part of which is provided as SEQ ID NO:11).

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide such as that provided as SEQ ID NO:22, SEQ ID NO:23 or SEQ ID NO:24. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides peptides of the invention or fragments thereof haptenised to another peptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

Preferably, antibodies of the present invention are detectably labeled. Exemplary detectable labels that allow for direct measurement of antibody binding include radiolabels, fluorophores, dyes, magnetic beads, cherniluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect polypeptides of the invention.

### Feedstuffs

The present invention includes compositions which can be used as feedstuffs. For purposes of the present invention, “feedstuffs” include any food or preparation for human or animal consumption (including for enteral and/or parenteral consumption) which when taken into the body (a) serve to nourish or build up tissues or supply energy; and/or (b) maintain, restore or support adequate nutritional status or metabolic function.

Feedstuffs of the invention comprise, for example, a cell of the invention, a plant or part thereof of the invention, a seed of the invention, or flour of the invention, along with a suitable carrier(s). The term “carrier” is used in its broadest sense to encompass any component which may or may not have nutritional value. As the skilled addressee will appreciate, the carrier must be suitable for use (or used in a sufficiently low concentration) in a feedstuff such that it does not have deleterious effect on an organism which consumes the feedstuff.

The composition may either be in a solid or liquid form. Additionally, the composition may include edible macronutrients, vitamins, and/or minerals in amounts desired for a particular use. The amounts of these ingredients will vary depending on whether the composition is intended for use with normal individuals or for use with individuals having specialized needs, such as individuals suffering from metabolic disorders and the like.

Examples of suitable carriers with nutritional value include, but are not limited to, macronutrients such as edible fats, carbohydrates and proteins. Examples of such edible fats include, but are not limited to, coconut oil, borage oil, fungal oil, black current oil, soy oil, and mono- and diglycerides. Examples of such carbohydrates include (but are not limited to): glucose, edible lactose, and hydrolyzed search. Additionally, examples of proteins which may be utilized in the nutritional composition of the invention include (but are not limited to) soy proteins, electrodialysed whey, electrodialysed skim milk, milk whey, or the hydrolysates of these proteins.

With respect to vitamins and minerals, the following may be added to the feedstuff compositions of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

The components utilized in the feedstuff compositions of the present invention can be of semi-purified or purified origin. By semi-purified or purified is meant a material which has been prepared by purification of a natural material or by de novo synthesis.

In one embodiment, the transgenic plant is a marigold which has increased levels of lutein production when compared to a wild-type plant, and the transgenic plant is used as a feedstuff for chickens for yellow yolks.

In another embodiment, the transgenic plant which has increased levels of lutein production when compared to a wild-type plant and is used to produce a feedstuff for use in aquaculture.

### EXAMPLES

### Example 1

### Materials and Methods

### Milling

Grain, for example from lines of the Sunco x Tasman doubled haploid population, was milled using a small-scale prototype mill (CSIRO Plant Industry). The milled grain was passed through a sieve of 0.160 mm diameter. This fraction was used for flour colour analysis and was free of bran and germ. Approximately 10 g of grain, when milled, yielded approximately 800 mg of fine white flour.

Wheat Pigment Extraction from Flour

Pigments were extracted from flour samples by vortexing 200 mg of flour with 500 μl of Acetone/Ethyl acetate (1.5:1) in an eppendorf tube for 2 min. Four hundred μl of H₂O was added, the sample vortexed for a further 1 min and then centrifuged at 13,000 rpm for 1 min. The upper ethyl acetate layer contained pigments and 120 μl was transferred to a HPLC vial with new Teflon liners to prevent evaporation.

### Saponification of Wheat Flour Carotenoids

Carotenoids were extracted from flour obtained from 6 g of grain using 15 ml acetone/ethyl acetate and 12 ml H₂O as described above. The extract (7 mls) was saponified by the addition of 10 ml of 50% (w/v) KOH in 80% (v/v) methanol. The sample was vortexed for 2 mins, centrifuged for 5 mins (13 000 g) and the supernatant retained. The pellet was resuspended in 10 ml of 50% (w/v) KOH in 100% methanol and ground. The sample was centrifuged as above and the supernatant retained. The supernatants were combined, and incubated at 37° C. for 4 hours. After incubation, 20 ml of diethyl ether and 20 ml H₂O were added and the solution was mixed. The mixture was centrifuged for 1 min (13 000 g) and the upper phase recovered. The upper phase was washed 3 times with H₂O (20 ml or an appropriate volume of H₂O was added, mixed, centrifuged as above, and the upper phase recovered). The upper phase was dried down under argon flow, with diethyl ether being added to remove water residue when necessary, and then resuspended in 200 μl ethyl acetate, of which 20 μl was used for HPLC analysis.

Wheat Pigment Extraction from Leaves

A 2 cm section of leaf was placed in a tube containing in 500 μl of Acetone/Ethyl acetate (1.5:1) and a 5 mm diameter ball bearing. The tissue was homogenised by shaking in a Qiagen Mixer Mill 300 for 30s at a frequency of 30 shakes per second. Four hundred μl of H₂O was added, the sample shaken for a further 30 seconds and then centrifuged at 13,000 rpm for 1 min. The upper ethyl acetate layer contained pigments and 120 μl was transferred to a HPLC vial with new Teflon liners to prevent evaporation.

HPLC analysis was the same as for the carotenoids extracted from flour.

### HPLC Analysis of Flour Carotenoids

Pigments were run on HPLC as described by Pogson et al., (1996). A Beckman system Gold 507e auto-sampler with a Waters spherisorb ODS2 column (5 uM packing) was used. Using a 168 photo-diode array detector, chromatograms were taken at 440 nm. A 20 μl injection volume and a 15 min run were used. HPLC analysis of pigments used an Acetonitrile (ACN): H₂O:Triethylamine (9:1:0.01) background solvent with a gradient solvent of Ethyl acetate (100%) at a 1.5 ml/min flow rate. The 15 min program began with a flow of background solvent only (100% ACN/H₂O). At 1 min, the gradient changed to 30% ethyl acetate for 4 min. At 5 min, the gradient changed to 40% ethyl acetate for 3 min. At 8 min, the gradient changed to 70% ethyl acetate for 1 min. At 9 min, 100% ethyl acetate was run through the column for 2.5 min. At 13.5 mins, the flow was returned to 100% background solvent for 1 min, the carotenoids having been separated. The column and detection system were then ready for the next sample.

RNA Isolation from Plant Tissues

RNA was isolated from endosperm of hexaploid wheat, for example from cultivars Chinese Spring, Sunco or Tasman, at 10 and 20 days post-anthesis (DPA). Samples which had been frozen on dry ice immediately after harvesting were stored at −80° C. until required. RNA was extracted from each tissue using the following protocol. Samples were weighed and ground in liquid nitrogen using a mortar and pestle. After the liquid nitrogen had evaporated but the ground tissue was still frozen, 600 μl of NTES (0.1 M NaCl, 10 mM Tris-HCl (pH 8.0) 1 mM EDTA, 1% (w/v) SDS, 2% (w/v) β-mercaptoethanol) and 800 μl phenol/chloroform for every 200 mg of tissue were added and tissue was ground for a further 5 min. The aqueous phase was recovered after centrifugation at 11,000 g for 10 min at 4° C. and the RNA precipitated at −20° C. overnight with an equal volume of 4 M lithium chloride/10 mM EDTA, pelleted and washed with 70% ethanol. The RNA was allowed to air dry in a laminar flow hood for 10 min. The pellet was then dissolved in 360 μl DEPC treated H₂O and the RNA re-precipitated using 1 ml 95-100% ethanol and 40 μl 2 M sodium acetate, pH 5.8. After centrifugation, the RNA pellet was dried, dissolved in 20 μl of DEPC H₂O and treated with 1 μl DNase I (Invitrogen). After inactivation of DNase by adding 5 μl of DNase inactivation agent and centrifugation for 1 min at 10,000 g, the concentration of RNA in the supernatant was measured from the absorbance of the sample at 260 nm.

### Reverse Transcription Reactions

Reverse transcription was carried out on the RNA samples using the Invitrogen Superscript II Reverse Transcription Kit as described by the supplier. First strand synthesis used 80 pmols of oligo d(T) primer to 1 μg of total RNA.

### Isolation of Wheat DNA

High quality genomic DNA was isolated from young hexaploid wheat plants for use in library construction as follows. Approximately 1 g of leaf tissue was ground up in liquid nitrogen using a mortar and pestle and then mixed with 4 ml DNA extraction buffer (1% Sarkosyl, 100 mM Tris-HCl pH 8.5, 100 mM NaCl, 10 mM EDTA and 2% PVP) and 4 ml phenol/chloroform (1:1) for 60 min at room temperature on an orbital rotor. The tubes were then centrifuged at room temperature for 10 min at 3,000 g, the supernatant treated with 40 μl of RNAse (10 mg/ml) at 37° C. for 30 min and extracted with 4 ml phenol/chloroform. The DNA was precipitated with 400 μl 3M sodium acetate, pH 4.8, and 4 ml ice-cold isopropanol, spooled, washed with 70% ethanol, dried and redissolved in 100 μl sterile dH₂O.

For PCR analysis, DNA was extracted from the leaves of plants using a FastDNA® kit (BIO 101 Inc., Vista, Calif., USA) according to the suppliers instructions. The DNA was eluted into 100 μl sterile deionized water and 1 μl used in PCR.

### Wheat Transformation

Transformed wheat plants may be produced using an efficient Agrobacterium-mediated seed inoculation method as follows. The method is at least as efficient as other reported methods for wheat.

Genetic constructs for transformation of wheat are introduced by electroporation into the disarmed Agrobacterium tumefaciens strain LBA4404 carrying the vir plasmid pAL4404 and pSB1, with subsequent selection on media with spectinomycin. Transformed Agrobacterium strains are incubated on solidified YEP media at 27° C. for 2 days. Bacteria are then collected and re-suspended in TSIM1 (MS media with 100 mg/l myo-inositol, 10 g/l glucose, 50 mg/l MES buffer pH5.5) containing 400 mM acetosyringone to an optical density of 2.4 at 650 nm for wheat inoculation.

Wheat plants (for example variety NB1, a Spring wheat variety, Nickerson Seeds Ltd, Rothwell, Lincs.) are grown in a glasshouse at 22/15° C. day/night temperature with supplemented light to give a 16 hour day. Tillers are harvested approximately 14 days post-anthesis (embryos approximately 1 mm in length) to include 50 cm tiller stem. All leaves are then removed from the tillers except the flag leaf, which is cleaned to remove contaminating fungal spores. The glumes of each spikelet and the lemma from the first two florets are then carefully removed to expose the immature seed. Generally, only these two seed in each spikelet are uncovered. This procedure is carried out along the entire length of the inflorescence. The ears are then sprayed with 70% IMS as a brief surface sterilization.

Agrobacterium suspensions (1 μl) are inoculated using a 10 μl Hamilton syringe into the immature seed approximately at the position of the scutellum:endosperm interface so that all exposed seed are inoculated. The tillers were then placed in water, covered with a translucent plastic bag to prevent seed dehydration, and placed in a lit incubator for 3 days at 23° C., 16 hr day, 45 μEm⁻² s⁻¹ PAR. After 3 days of co-cultivation, the inoculated immature seed are removed and surface sterilized with 70% ethanol (30 sec), then 20% bleach (Domestos, 20 min), followed by thorough washing in sterile distilled water. Immature embryos are aseptically isolated and placed on W3 media (MS supplemented with 20 g/l sucrose and 2 mg/l 2,4-D and solidified with 6 g/l Type I agarose, Sigma) with the addition of 150 mg/l Timentin (W3T) and with the scutellum uppermost (20 embryos per plate). Cultures are placed at 25° C. in the light (16 hour day, 80 μEm⁻² s⁻¹ PAR). The development of the embryonic axis on the embryos is assessed about 5 days after isolation and the axis removed where necessary to improve callus production. The embryos are maintained on W3T for 4 weeks, with a transfer to fresh media at 2 weeks post-isolation, and assessed for embryogenic capacity.

After 4 weeks growth, callus derived from the inoculated embryos is very similar to control callus obtained from uninoculated embryos plated on W3T medium. Presence of the bacteria does not appear to have substantially reduced the embryogenic capacity of the callus derived from the inoculated embryos. Embryogenic calli are transferred to W3 media with 2 mg/l Asulam or geneticin at 25 mg/l as selective agent, as appropriate, and 150 mg/l Timentin (W32AT). Calli are maintained on this media for a further 2 weeks and then each callus is divided into 2 mm-sized pieces and re-plated onto W32AT. Control embryos derived from inoculations with the LBA4404 without binary vector constructs do not produce transformed callus on selection media.

After a further 2 weeks culture, all tissue is assessed for development of embryogenic callus: any callus showing signs of continued development after 4 weeks on selection is transferred to regeneration media (RMT-MS with 40 g/1 maltose and 150 mg/l Timentin, pH 5.8, solidified with 6 g/l agarose, Sigma type 1). Shoots are regenerated within 4 weeks on this media and then transferred to MS30 with 150 mg/l Timentin for shoot elongation and rooting. Juvenile plants are then transferred to soil mixture and kept on a misting bench for two weeks and finally transferred to a glasshouse.

Alternative Agrobacterium strains such as strain AGL1 or selectable markers such as genes encoding hygromycin resistance can also be used in the method.

### Genome Walking

A description of the genome walking technique can be found in Rishi et al. (2004).

Genome walking was carried out to obtain the full genomic DNA sequence for the wheat epsilon cyclase gene using the Universal GenomeWalker™ Kit (BD Biosciences). Genomic DNA was extracted using the high-quality DNA extraction protocol outlined above. gDNA (2.5 μg) was digested in 4 separate reactions by 4 different restriction enzymes, DraI, EcoRV, PvuII and StuI. Digestion reactions contained 10 μl of restriction enzyme buffer, 8 μl of restriction enzyme, 2.5 μg gDNA and d H₂O to a final volume of 100 μl. Digestion proceeded for 2 hrs at 37° C., after which the reactions were vortexed and allowed to continue to digest at 37° C. for a further 16-18 hrs. Digestion reactions were purified by phenol/chloroform extraction and precipitation of the DNA with ethanol. After the DNA recovered, it was dissolved in 20 μl of TE (10/0.1, pH 7.5) for use in library construction. Each restriction digest generated a different family of genomic fragments, which were ligated to the GenomeWalker Adapter to create a library of gDNA fragments. From each digest, 4 μl of digested, purified gDNA was ligated to 1.9 μl of GenomeWalker adaptor using 1.6 μl 10× ligation buffer and 0.5 μl T4 DNA ligase. Ligation reactions were left at 16° C. overnight. The reactions were stopped by incubation at 70° C. for 5 mins, then 72 μl of TE (10/1, pH 7.4) was added and the tubes vortexed slowly for 10 seconds. The libraries (1 μl of each) were then used in the primary PCRS.

### Embryo Excision and Biolistic Transformation

Transformation of wheat embryos was performed according to the method of Pellegrineschi et al. (2002). Approximately one hundred seeds were removed from heads of hexaploid wheat at approximately 10 days post anthesis and sterilised, by washing seed in 50 ml of 10% (v/v) bleach for 20 mins, followed by rinsing with sterile water. The embryo from each seed was removed, the axis excised and the embryos placed scutellum down on osmotic media (or wheat MSM). Approximately 30 embryos were placed within a 2×2 cm square in the centre of the plate. The 30 embryos were allowed to remain untouched for a period of no shorter than 2 hrs and no longer than 4 hrs.

While the embryos were on osmotic medium, the gold/DNA mixture was prepared. A 50 μl aliquot of gold was sonicated for 2 mins before the addition of 5 μg of plasmid DNA. Plasmid DNA was a mixture of the RNAi encoding plasmid and the resistance plasmid pNeo in a ratio of 1:1. The mixture was vortexed briefly then 50 μl of 2.5 M CaCl₂ and 20 μl 0.1 M spermidine were placed into the lid of the tube before vortexing into the gold to precipitate the DNA onto the gold. The sample was centrifuged for 5 seconds, the supernatant removed, 150 μl of 100% ethanol added and the gold mixture resuspended. The spin was repeated, and again the supernatant was removed, before the gold mixture was resuspended in 85 μl of ethanol in preparation for use in embryo bombardment.

Ten μl of the gold mixture was used in each bombardment except for the one plate of embryos left un-bombarded to act as a regeneration control. Bombardment was carried out at 900 psi at a distance of 9 cm. After bombardment, plates containing embryos were placed in the dark at 26° C. After 24 hours, embryos were transferred face down to induction media (or wheat MSR), which contained 2,4D to induce callus formation, and once again plates were placed in the dark at 26° C.

After 2 weeks the callus had developed and was transferred to maturation media (or wheat MSW), with antibiotic selection, to allow for the development and differentiation of transformed plant tissue. Plates were placed in the light, at the constant temperature of 26° C., and were left undisturbed for up to three weeks. During this time greening of some callus occurred, and after 3 weeks green callus was transferred to fresh media to allow for further plant development. Typically, plants were transferred another two times (6 weeks) before being planted into soil or vermiculite in the greenhouse.

### Screening of Putative Transformed Wheat Plants

Leaf DNA was extracted using the fast DNA extraction kit and each individual plant screened for the presence of the epsilon cyclase duplex by PCR analysis. A leaf sample was taken from an untransformed Bob White plant to act as a PCR control. To screen putative transgenics for the pStarling/ε-cyclase construct, the primer pStarF (5′ ATGATGGCATATGCAGCAGC 3′) (SEQ ID NO:69) was used in conjunction with ECYCR/BsiWI (5′ GATGACATGGCCATGGCATA 3′) (SEQ ID NO:31). To screen putative transgenics for the pBx17/ε-cyclase construct, primers specific to the Rint9 intron, ScrF (5′CACCTTCGATACAAGGTTGTGC 3′) (SEQ ID NO:32) and ScrR (5′ ACAAATTGCAGAGCTTGACTGC 3′) (SEQ ID NO:33) were used in PCR of plant gDNA. Transgenic plants containing the pBx17/ε-cyclase construct generated a PCR product.

### Example 2

### Wheat Flour Carotenoid Analysis by HPLC

In the past, a variety of carotenoid extraction methods with varying amounts of seed material had been used for carotenoid analysis by HPLC. For example, Ward et al. (1997) extracted 20 g of wheat flour with hexane and diethyl ether before filtering, drying and resuspending pigments in 5 ml acetone. Hentschel et al., (2002) extracted 2 g of durum wheat semolina with methanol/tetrahydrofuran (1:1) and after evaporation, redissolved the residue in a suitable quantity of methanol/tetrahydrofuran for HPLC analysis. A standardised carotenoid pigment extraction for wheat uses 8 g of wheat flour, which is extracted with 40 ml water-saturated butanol (Kaneko et al., 1995; AACC, 2000). These methods use relatively large sample sizes and large amounts of organic solvents for complete extraction and have a time-consuming step of solvent evaporation or paper filtration, making them less suitable for analysis of large numbers of samples. Some published methods are listed in Table 2.

To develop a simpler and more rapid carotenoid extraction method from wheat flour using minimum sample sizes, some of these methods were scaled down and compared. The AACC (2002) method was modified by using as little as 1 g of hexaploid wheat flour with 1 ml of solvent. Carotenoids were also extracted following the method of Ye et al., (2000) using 1 g of seed material with 4×1 ml extractions with acetone. The residue was evaporated under argon gas and the residue resuspended in chloroform for analysis by HPLC. Lutein pigments were identified by HPLC in extracts of wheat flour by both methods. As a comparison, the method for pigment analysis of leaf tissue (Pogson et al., 1996) was tested and applied to wheat flour extraction. Initially, sample sizes of 2 g of flour and 10 ml of acetone/ethyl acetate were used. However after testing sample sizes ranging from 100 mg to 1 g of flour with corresponding amounts of solvent, it was found that as little as 200 mg of hexaploid wheat flour could be fully extracted with 500 ul of acetone/ethyl acetate to produce an extract with detectable levels of carotenoids suitable for HPLC analysis.

TABLE 2
Methods for extracting carotenoids from seed.

| | Amount of | | | Volume of | Additional |
|---|---|---|---|---|---|
| Method | Flour | | Solvent | Solvent | Steps Required |
| Ward et al., | 20 | g | Hexane, diethyl | Unpublished, at | Evaporation and |
| (1997) | | | ether | least 20 ml | resuspension |
| | | | | required | |
| Hentschel et al., | 2 | g | Methanol, | Unpublished, at | Evaporation and |
| (2002) | | | tetrahydrofuran | least 2 ml | resuspension |
| | | | | required | |
| AACC (2002) | 8 | g | Water-saturated | 40 ml | Paper filtration |
| | | | butanol | | |
| Ye et al., (2000) | 1 | g | Acetone | 4 × 1 ml | Evaporation and |
| | | | | extractions | resuspension |
| Pogson et al., | 200 | mg | Acetone/ | 500 μl | Centrifugation |
| (1996) modified | | | ethylacetate, water | | for 1 min |
| for wheat flour | | | | | |

The carotenoid content of 100 mg of flour tended to be insufficient for detection, while the extraction of 300 mg of flour with 500%1 of solvent did not completely extract the carotenoids from the flour sample. Using 200 mg of flour with the method of Pogson et al., (1996), multiple solvent extractions and evaporation and residue resuspension steps were not required. Furthermore, the extract containing carotenoids was clean of any flour reside due to pigments moving into the upper phase, away from flour particles, which centrifuged to the bottom of the tube. Additionally, the ease of sample processing meant reproducibility was high and comparable quantification of samples could be carried out with confidence. This method was therefore used in the following analyses and serves as a standard method.

Whole-wheat grains were milled and sieved to 0.160 mm as described in Example 1 to obtain the flour fraction consisting of endosperm only. Carotenoids were extracted as described above from 200 mg samples, producing a visibly obvious yellow pigment in the upper ethyl acetate fraction. Samples were run on HPLC as described in Example 1, with a background solvent of Acetonitrile (ACN):H₂O:Triethylamine (9:1:0.01) and a gradient solvent of Ethyl acetate (100%) which separated the yellow pigments according to hydrophobicity. As pigments were separated on the column, each individual carotenoid was detected by the photo diode array, the spectrum obtained and a retention time assigned. Retention times and spectra of carotenoids present in wheat flour extracts were compared to known carotenoid retention times and spectra identified in leaf extracts (Pogson et al., 1996).

Retention times and spectra of wheat flour extracts demonstrated that lutein was the main carotenoid present in durum wheat flour and bread wheat flour. However, bread wheat flour also contained apparently late-eluting carotenoids that could not be identified by simple comparison to standard leaf carotenoids (FIG. 2). The late-eluting carotenoid peaks seen in the bread wheat flour extract had a retention time of β-carotene, yet displayed lutein spectra.

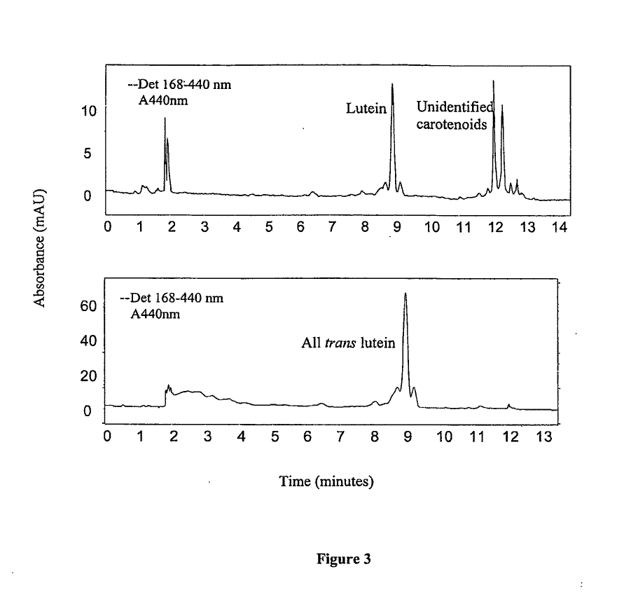

While some studies have reported β-carotene in flour (Ward et al., 1997) previous studies had also reported the presence of lutein esters in wheat flour (Lepage and Sims, 1968). To investigate the possibility that the late-eluting peaks were lutein esters, a saponification reaction was carried out on the bread wheat flour extract, as described in Example 1. Saponification involved alkaline treatment and hydrolysis of any fatty acid esters of lutein, converting them to free lutein and fatty acids. The HPLC traces for such treated samples did not show the late eluting peaks (FIG. 3). This confirmed that the bread wheat flour contained lutein in the form of esters, along with free lutein, and did not contain β-carotene.

### Example 3

### Investigation of Wheat Flour Carotenoids during Grain Development

### Results

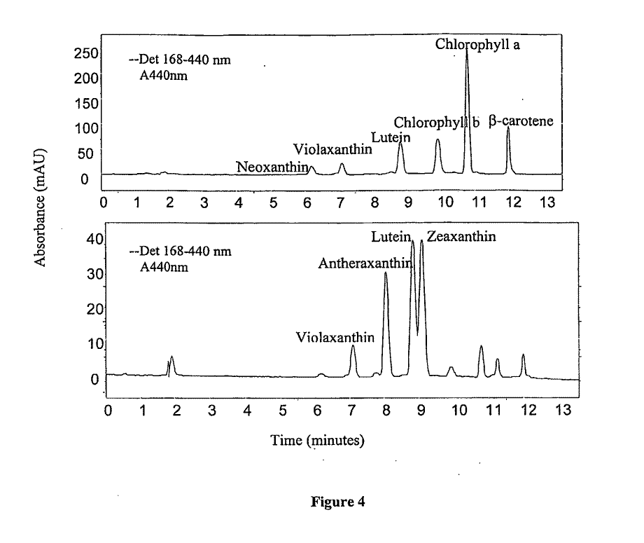

The carotenoid content of developing wheat grain, obtained from a Sunco x Tasman doubled haploid population (see below), was investigated by extracting carotenoids from endosperm at 10, 15, 20, 30 and 40 days post-anthesis (DPA) using the above described method and analysing extracts by HPLC. At 10 DPA, the endosperm was easily isolated from the green seed coat by pinching the immature seed. The green seed coat had a carotenoid content typical of photosynthetic tissue whereas the endosperm was noticeably bright yellow and, surprisingly, contained many carotenoids (FIG. 4) including violaxanthin, antheraxanthin, zeaxanthin and lutein. As little as 150 mg tissue was sufficient to obtain the profile and spectra of all carotenoids present. Two of the carotenoids were not present in the standard leaf carotenoid profile. The carotenoid eluting at 8 minutes was thought to be antheraxanthin due to elution between violaxanthin and lutein, and this was confirmed by comparison of the spectrum with that of known antheraxanthin. Likewise, the peak eluting immediately after lutein was identified as zeaxanthin by comparison of its spectrum to standard zeaxanthin.

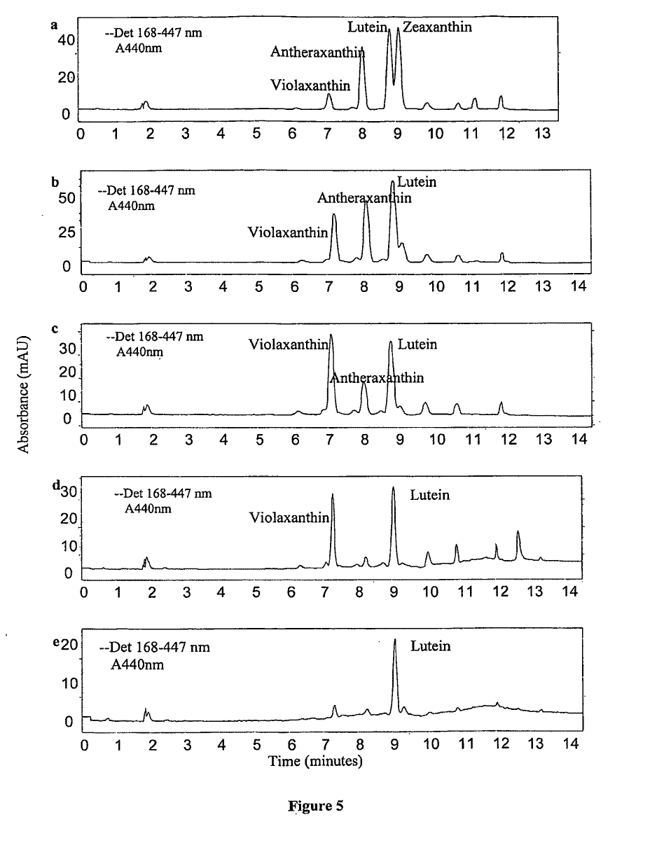

At 15 DPA, there was little change in endosperm carotenoid profile compared to 10 DPA except for loss of most of the zeaxanthin (FIG. 5, panel b). At 20 DPA, the endosperm appeared white to the eye when compared to endosperm from earlier points of development and HPLC analysis showed that antheraxanthin levels were reduced (FIG. 5, panel c). At 30 DPA, the green outer layer of the grain was starting to fade to yellow, the endosperm was milky-white and sticky and the embryo was more noticeably yellow. Carotenoid analysis at 30 DPA showed the endosperrn contained violaxanthin and lutein only (FIG. 5, panel d). By 40 DPA, the endosperm properties were like flour in that when water was added in the carotenoid extraction protocol, a doughy substance formed. Carotenoid analysis at this point showed a loss of violaxanthin, with the remaining pigment being lutein only (FIG. 5, panel e). At grain maturity, when the wheat plant and grain were no longer green, seed was harvested, threshed and completely dried for 2 days at 37° C. Whole grains were crushed and analysed for pigments and mature grain contained lutein in the form of free lutein only. The petals of wheat flowers, taken early after anthesis, were also analysed for carotenoids, and were found to contain mainly lutein esters in a profile similar to that of marigold flowers.

Total carotenoid levels in 20 endosperm from each of 10, 20 and 30 DPA developing seeds were measured and found to be 590 μg, 390 μg and 200 μg respectively. This showed that the total carotenoid content in endosperm decreased through development as well as the observed loss of diversity. However, the lutein levels surprisingly remained relatively constant during endosperm development.

### Discussion

Carotenoids were present in the developing wheat grain in both the endosperm and bran layers from as early as 10 DPA. At this stage, the yellow colour of the endosperm was due to at least four different carotenoids: violaxanthin, antheraxanthin, lutein and zeaxanthin. The carotenoids present in the bran layer were typical of photosynthetic tissue and probably represented carotenoids localised within chloroplasts. During the development of wheat endosperm, there was a reduction of total carotenoid levels as the zeaxanthin, antheraxanthin and finally violaxanthin pigments were lost from the endosperm, but the lutein remained present at relatively constant levels.

At maturity, the only carotenoid in both endosperm and the bran layer was lutein. Lutein may remain to protect the seed and membranes from photo-oxidation and deterioration by limiting the amount of free radicals.

The loss of the other carotenoids from the endosperm during development may be due to catabolism of violaxanthin by cleavage enzymes leading to ABA production. ABA levels in developing cereal grains increase steadily from 20 DPA until 40 DPA, increasing about 60-fold, and then decrease back to initial levels at 60 DPA (King, 1976). It seems that ABA accumulation (and resulting dormancy) was mirrored by reduction in carotenoids in wheat grain.

### Example 4

### Quantification of Wheat Flour Lutein by HPLC

### Results

The method described above to quantify lutein in different wheat grains was based on the observation that lutein (including lutein esters) was the predominant carotenoid in mature wheat endosperm. In order to quantify lutein, areas under the lutein peak on HPLC traces, calculated by HPLC software, were interpolated on a standard curve to determine the lutein content in micrograms per gram of flour. In order to develop the standard curve, purified lutein samples, obtained from HPLC-separated leaf carotenoids, were resuspended in a given volume and the absorbance read at 445 nm. The absorption coefficient at this wavelength (E 1 cm, 1%) of a 1% solution of lutein in ethylacetate for a 1 cm light-path was taken as 2550 (Gilmore and Yamamoto, 1991). A dilution series ranging from 500-50,000 ng of lutein was then run on HPLC to obtain peak areas for each lutein amount. The data was graphed to produce the standard curve, and from this, an equation could be calculated, which calculates lutein content (μg) from HPLC peak area:

$$y μgs of lutein = \frac{9.498 \times {10}^{- 5} \times peak area}{1000}$$

Adjusting for the amount of flour used and the fraction of sample analysed, the total amount of lutein in 1 g of flour could be calculated using the following equation:

y μgs lutein×(175/20)×5=μg lutein (g flour)⁻¹

In this analysis, the peak area of lutein and lutein esters were combined in calculations of lutein content. Lutein and lutein esters have the same absorbance spectrum (maxima at 424.2, 446 and 478.5 nm) and hence a lutein standard curve could be used to quantitate lutein esters (Piccaglia et al., 1998).

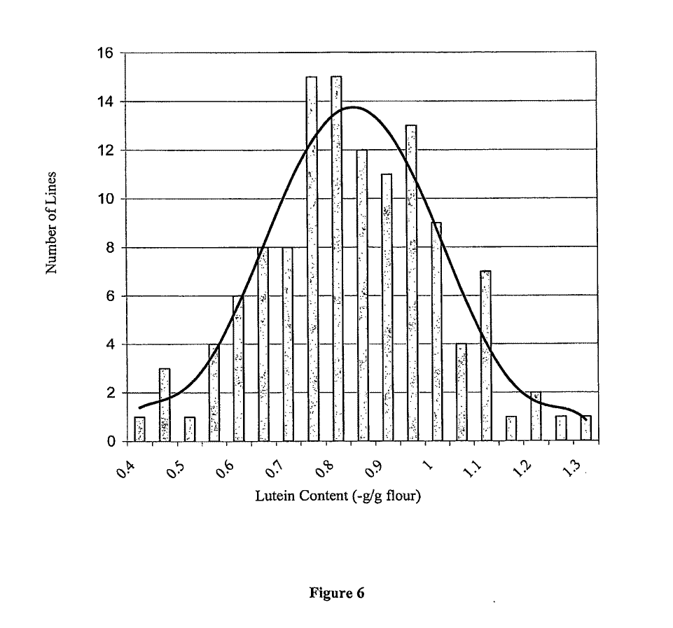

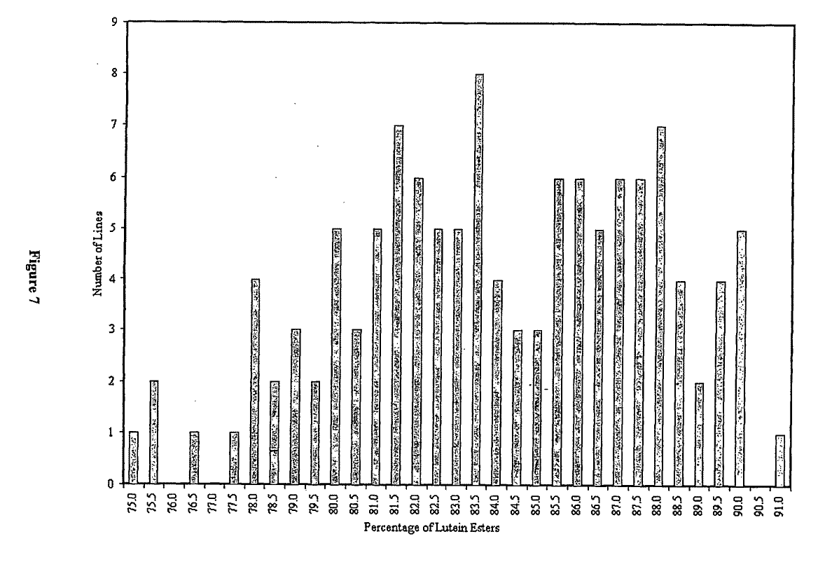

Lutein levels were measured in flour from lines of a Sunco x Tasman doubled haploid population grown in Roseworthy in 1999. Milling and duplicate flour lutein measurements were taken for 122 individual lines. Values obtained for flour lutein content ranged from 0.38 to 1.28 micrograms of lutein per gram of flour. Continuous variation between these extremes was observed, as expected for a multilocus quantitative trait resulting from a number of independent random events. The lutein data also shows the expected normal distribution curve for a segregating quantitative trait (FIG. 6) with the bulk of values falling in the mid-range. The lutein data was used to determine lutein QTLs as described below.

All of these samples showed a high proportion of lutein esters. No esters were present in fresh grain and it is proposed, in agreement with Kaneko et al., (1995) that esters were produced over time, with ageing of the grain. There has been some evidence of enzymically controlled carotenoid esterification in plant tissues and it was hypothesised that ester formation in wheat grain might be enzymically controlled. To determine the underlying genetic control on the formation of lutein esters in wheat grain, the lutein ester percentages were investigated for later use in QTL analysis. The percentage of endosperm lutein that exists as lutein esters was calculated for each of the lines by dividing the amount of lutein esters by the total lutein content. The mean percentage of lutein esters for the 122 individual wheat lines was 83% of the total lutein content. The range of values was from 75% to 91%. When the lutein esters percentages were graphed as a histogram, the distribution of lutein esters in the Sunco x Tasman doubled haploid population did not show a normal distribution; rather the distribution seemed to be a mixed normal distribution, containing 2 population ranges of relatively low or high lutein ester percentages (FIG. 7). This suggests that genotype plays a role in ester production.

The cultivar Pandas was determined to have a grain lutein content between that of Sunco and Tasman.

### Discussion

The accurate analysis of wheat flour carotenoids described here conclusively showed lutein and lutein esters to be the major determinants of wheat flour colour and suggested that previous claims of β-carotene in wheat flour (Ward et al., 1997) were based on misidentification of a lutein ester that displayed a β-carotene retention time. Flour from freshly threshed grain had a total lutein content that was all trans-lutein while stored grain contained a high proportion of lutein esters, indicating that lutein esters were produced during storage and ageing of the grain. Esterification is known to be a common means to sequester carotenoids in fruits and flowers but has not been shown previously for wheat flour.

Lutein has been predominately detected along with trace levels of zeaxanthin and isolutein in a front-surface absorbance analysis of nine different wheat flours (Zandomeneghi et al., 2000). Lutein was shown to be the main carotenoid in wheat being distributed through the grain (Panfili et al., 2004). That study also identified zeaxanthin, α-carotene and β-carotene in wheat samples, albeit at much lower contents than lutein. However, the observed presence of these carotenoids was most likely due to the fraction of flour used in that analysis, having been sieved through a 0.5 mm sieve and thus containing a noticeable proportion of bran and contaminating zeaxanthin-rich germ. In contrast, the current study used the flour fraction sieved to 0.16 mm for carotenoid analysis and thus did not contain the zeaxanthin and α+β-carotene-rich germ, hence the absence of these carotenoids.

### Example 5

### QTL Analysis of Lutein Content in Wheat Flour

### Results

MapManager QTX software (www.mapmanager.org) was used to analyse the lutein content data from the Sunco c Tasman doubled haploid population of 122 lines for potential QTLs associated with lutein content. The Sunco c Tasman AC38 progeny set are part of the National Wheat Molecular Marker Program in Australia, consisting of 178 individual genetic maps. The Kosabi map function (default function for QTL marker regression) was used and marker regression was carried out for chromosomes 1A to 7D.

Primary associations were evaluated testing a 0.05 significance level. QTLs were shown on chromosomes 3B, 5B and 7A and smaller associations were shown on 1A, 2B, 4B, 4D, 5A and 6B. To reveal the most significant associations, marker regression was performed using a 0.001 significance level. Chromosomal regions on 3B, 5B and 7A proved significant for lutein association. Interval mapping for the lutein trait was also performed for chromosomes 3B, 5B and 7A to depict the locations of these QTLs schematically.

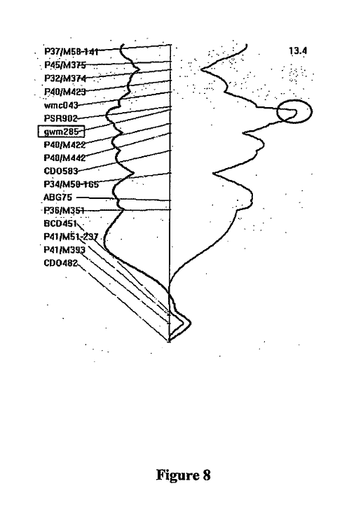

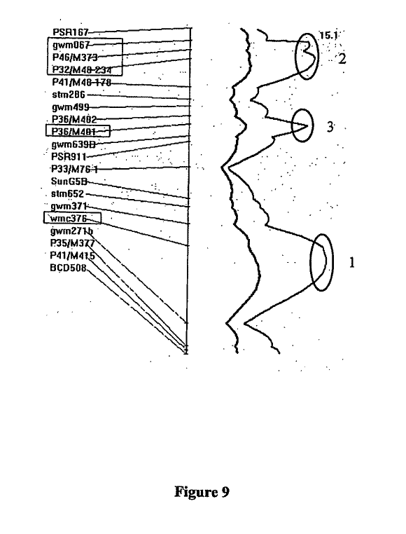

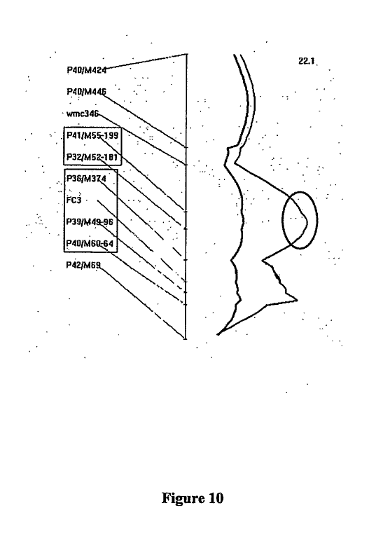

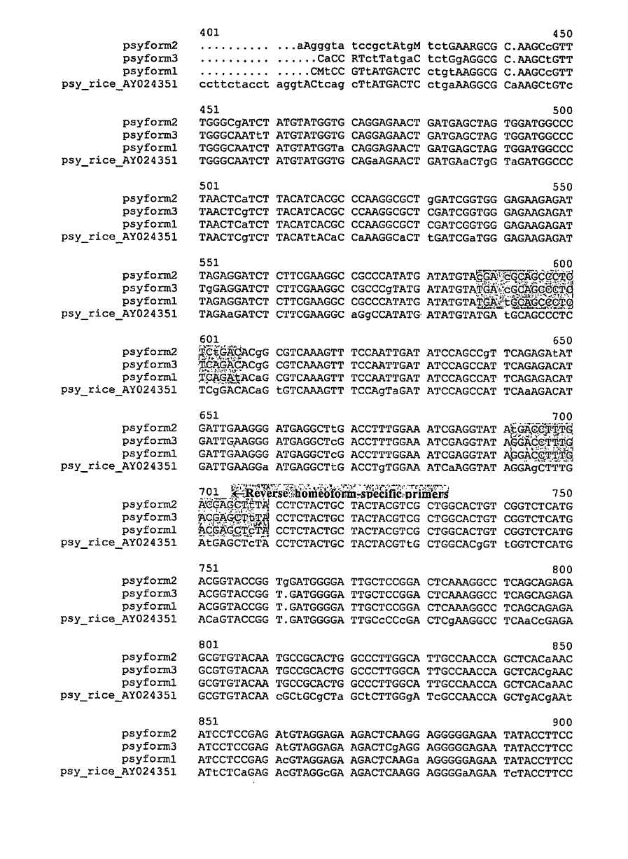

The QTL on 3B (FIG. 8) was closest to marker gwm285, most likely on the short arm of chromosome 3B, and accounted for ˜9% of the trait variation. Three different regions, potentially 3 different QTLs, were identified on 5B (FIG. 9). The most significant of these was closely associated to marker wmc376. The second QTL on 5B was in the region of markers gwm067, P46/M373 and P32/M48-234. The third QTL on 5B was most closely associated with the marker P36/M401. Each of the 5B QTLs accounted for ˜10% of the lutein variation. The QTL on 7A (FIG. 10) was most closely linked to the marker P41/M55-199, and accounted for 17% of the lutein variation. With regard to FIGS. 8 to 10, the LOD scores refer to the likelihood of association and LOD scores >10 were significant for association with lutein accumulation.

QTL analysis for the lutein ester data was also performed using MapManagerQTX. Marker regression analysis was carried out on the putative quantitative trait of percentage of lutein esters for chromosomes 1A to 7D of the Sunco x Tasman AC38 set. Regression analysis carried out at the 0.0001% significance level, using the Kosabi map function, showed significant association of the trait to chromosome 2B. The 2B association was examined using interval mapping and the Haldane map function, once again with the linkage criterion using the 0.0001% significance level. The LOD score for association of lutein ester percentage to chromosome 2B was 31.9, which was much higher than the lutein data associations and indicated a major gene of impact on chromosme 2B that contributed to the formation of lutein esters in wheat endosperm. The 2B QTL was associated with many markers along 2B, making the region of the QTL quite widespread compared to the lutein QTLs identified on 3B, 5B and 7A.

### Discussion

The QTLs on chromosomes 3B, 5B and 7A were in agreement with previous QTLs identified for flour b* values and xanthophyll content (Mares and Campbell, 2001). Spectrophometric measurements of total xanthophylls in wheat flour have also highlighted regions on 3B and 7A as colour QTLs, but did not detect the QTL at 5B (Francki et al., 2004).

The QTL on 5B detected by both the b* and HPLC measurements was proximal to the centromere on the short arm of 5B, and the 2 additional QTLs identified by the HPLC method were distal to the centromere on the short arm of chromosome 5B and on the long arm of 5B, respectively (FIG. 9). The QTL on 7A detected by all three methods was distally placed on the long arm of chromosome 7A (FIG. 10). Three doubled haploid wheat populations, Schomburgk x Yarralinka, Cranbrook x Halberd, and Sunco x Tasman have a QTL for flour colour in the same region on chromosome 7A. Halberd, Tasman and Schomburgk are the source of the yellow allele in these populations, yet there is no common parent in the pedigree of the three cultivars (Parker et al., 1998) suggesting the general significance of this region for flour colour (Mares and Campbell, 2001).

The QTL on 7A had a higher impact on colour variation than the 3B QTL. In xanthophyll measurement methods, where extracts are quantified by spectrometery, the QTL on 3B accounted for 20% of the variation, whereas the QTL on 7A accounted for 27% of the variation for flour colour in Sunco x Tasman (Mares and Campbell, 2001). In the current study, the QTL on 3B determined 9% of the variation, and the QTL on 7A, 17%. The QTLs on 3B and 7A have also been identified in the Cranbrook x Halberd population, in the same regions of chromosomes 3B and 7A as Sunco x Tasman (Mares and Campbell, 2001). However the QTLs on 3B were not identified in the CD87 x Katepwa (Mares and Campbell, 2001) and Schomburgk x Yarralinka (Parker et al., 1998) populations. Rather, both populations had a QTL on 3A and the CD87 x Katepwa 3A QTL was in the region homeologous to the QTL on 3B in Sunco x Tasman (Mares and Campbell, 2001). It seems the activity of the underlying gene homeoforms on 3A and 3B can vary in different populations, explaining the varying contribution of homeologous regions on different genomes to overall flour colour.

### Example 6

### Identification of Candidate Genes Determining Flour Colour from Wheat

The rice genome has been sequenced and is sometimes useful as the reference genome for gene order in other cereals such as maize, wheat and oats. This conservation of gene order between species is known as synteny. However, it is also well documented that the syntenic relationship between wheat and rice is sometimes broken (for example see Li et al., 2004). The inventors decided to examine the rice genome for carotenoid biosynthesis genes and determine whether any of the rice sequences might correspond, based on synteny between the cereals, with the wheat regions containing the flour colour QTLs, which could then be examined for candidate genes.

Rice chromosomal locations of carotenoid biosynthesis genes were identified in the rice genomic sequence (www.tigr.org). Chromosomal locations in rice were determined for geranylgeranyl pyrophosphate (GGPP) synthase, isopentenyl pyrophosphate (IPP), phytoene synthase (PSY), phytoene desaturase (PDS), β-cyclase, ε-cyclase, β-carotene hydroxylase (βOH), ε-carotene hydroxylase (εOH), zeaxanthin epoxidase (ZE) and violaxanthin de-epoxidase (VDE) and are listed in Table 3. The cereal synteny chart displaying cereal genomes aligned on the common reference loci of rice (Gale and Devos, 1998) was used to predict the chromosomal location of the corresponding wheat genes, also shown in Table 3.

TABLE 3
Rice-wheat synteny for carotenoid gene locations. The table shows the
chromosomal location of rice carotenoid biosynthetic genes and the
predicted corresponding chromosomal location in wheat.

| Carotenoid Enzyme | Rice Chromosome | Wheat Chromosome |
|---|---|---|
| Geranylgeranyl | 5 | 1 |
| pyrophosphate (GGPP) | 7 | 2 |
| synthase | | |
| Isopentenyl pyrophosphate | 5 | 1 |
| (IPP) | 7 | 2 |
| Phytoene synthase (PSY) | 6 | 7 |
| | 9 | 5L |
| | 12 | 5S |
| Phytoene desaturase (PDS) | 3 | 4 or 5L |
| β-cyclase | 2 | 6 |
| ε-cyclase | 1 | 3 |
| ε-carotene hydroxylase | 3 | 4 or 5L |
| (εOH) | | |
| β-carotene hydroxylase | 4 | 2 |
| (βOH) | 10 | 1 |
| Zeaxanthin epoxidase (ZE) | 4 | 2 |
| Violaxanthin de-epoxidase | 4 | 2 |
| (VDE) | | |

Four different carotenoid genes were comparatively mapped to wheat chromosomes containing QTLs for lutein content. Of the 3 PSY loci in rice, 2 loci were comparatively mapped to chromosome 5 in wheat and 1 locus was comparatively mapped to chromosome 7 in wheat. The PSY isoforms located on chromosomes 5S, 5L and 7 and potentially underlie the observed QTLs for lutein content on 5B and 7A. PDS and εOH also were predicted to be located on chromosome S in wheat and may also underlie the observed QTLs for lutein content on 5B. The gene encoding ε-cyclase was comparatively mapped to chromosome 3 in wheat and potentially underlies the observed QTL for lutein content on 3B. Of these, we considered PSY and ε-cyclase to be the more likely candidates underlying lutein QTLs.

To obtain wheat sequences for carotenoid biosynthesis genes expressed in endosperm, RNA was extracted from developing endosperm (cv. Chinese Spring) at either 10 or 15 DPA and reverse transcribed into cDNA for use in PCR. The primers used for the PCR reaction were designed on the basis of conserved regions that were identified in genes encoding PSY or ε-cyclase biosynthetic enzymes in other plant species and are shown in Table 4.

TABLE 4
List of primer sequences.

| | |
|---|---|
| Primer Name | Primer Sequence (5′ to 3′) |

| | | |
|---|---|---|
| PSYFOR | AGTACGCCAAGACCTTCT | (SEQ ID NO:34) |
| PSYREV | GTGAAGTTGTTGTAGTCG | (SEQ ID NO:35) |
| epsilonf | GGGAGGATGAATTCAAAG | (SEQ ID NO:36) |
| epsilonr | CCACATCCATTTGGGCAA | (SEQ ID NO:37) |
| ECYCF/INTRON/ | CATTGTTGCATCTGGAGCAG | (SEQ ID NO:38) |
| BsiWI | | |
| ECYCR/BsiWI | GATGACATGGCCATGGCATA | (SEQ ID NO:31) |
| psyform1f | TGATGCAGCCCTCTCAGAT | (SEQ ID NO:39) |
| psyform2f | GGACGCAGCCCTCTCATAC | (SEQ ID NO:40) |
| psyform3f | TGACGCAGCCCTCTCAGAC | (SEQ ID NO:41) |
| psyform1r | TAGAGCTCGTCAAAGGTCC | (SEQ ID NO:42) |
| psyform2r | TAAAGCTCGTCAAAGGTCA | (SEQ ID NO:43) |
| psyform3r | TAAAGCTCGTCAAAGGTCC | (SEQ ID NO:44) |
| ecycform1F | GTATATTGTGGAAGGGGCCA | (SEQ ID NO:45) |
| ecycforin2F | GTATATTGTGAAAGGGACCG | (SEQ ID NO:46) |
| ecycform3F | GTATATTGTGGAAGGGGCCG | (SEQ ID NO:47) |
| ecycform1R | AAAACCATTAAGCTGGGATC | (SEQ ID NO:48) |
| ecycform2R | AAAACCATTAAGCTGGGGTT | (SEQ ID NO:49) |
| ecycform1ar | GAACTGGTGCAGAAACATCC | (SEQ ID NO:50) |
| walk1f1 | CAAGAACGGAAACGTCAGCGCTCATTC | (SEQ ID NO:51) |
| walk1f2 | CTTTGGATTGGCCTTGATAATTCAACTG | (SEQ ID NO:52) |
| walk1r1 | GCTCGGCCAATCATTATCGGCTTGTTAC | (SEQ ID NO:53) |
| walk1r2 | ACACGACAGTATCCTTCCATACATGCTC | (SEQ ID NO:54) |
| walk2f1 | GCAATGATCAGGACCTACCTGACCTTG | (SEQ ID NO:55) |
| walk2f2 | GTCTGCGAGAAATCTAGAAAGTGTACAG | (SEQ ID NO:56) |
| walk2r1 | TAATCAACTACTTGAAGGATGCTACCAGAC | (SEQ ID NO:57) |
| walk2r2 | GAAGTTTCATCAGCTATAGGCAACAGCTGC | (SEQ ID NO:58) |
| Intron1F | GGAGATCGTACAGACCGAGCGG | (SEQ ID NO:59) |
| Intron1R | CATCAGCTATAGGCAACAGCTGC | (SEQ ID NO:60) |
| RTECYCR | ATACGAACTCCCATCGCATC | (SEQ ID NO:61) |
| IntraF | GCTTTTTCCGGTTGCCCAAATGG | (SEQ ID NO:62) |
| IntraR | GGTGCTCATCGGGACTCAAGG | (SEQ ID NO:63) |

### Wheat PSY Genes

In rice, 3 isoforms of PSY have been identified, encoded by genes on chromosomes 6, 9 and 12. Therefore it was possible that 9 expressed mRNAs for PSY could be amplified from wheat cDNA, encoded by three PSY genes at distinct loci (‘isoforms’) with three homologs at each locus (from the A, B and D genomes) encoding PSY ‘homeoforms’. Two primers, PSYFOR and PSYREV (Table 4), were designed on the basis of conserved regions in the known or possible PSY genes from capsicum (Accession No. X68017), mandarin (AB037975), Arabidopsis (BT000450), a partial wheat EST (CA616424), rice (AY024351) and maize (U32636). PCR reactions using these primers and the wheat endosperm cDNA as template generated a 750 bp PCR product. The PCR products were cloned into pGEM-T and 20 clones were sequenced.

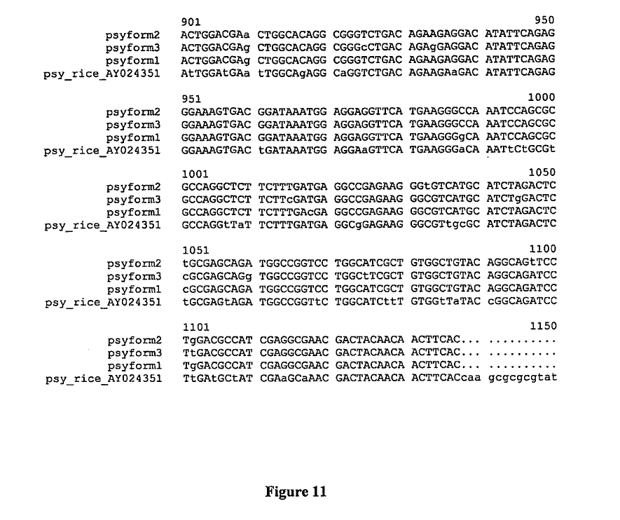

Analysis of the three sequences obtained showed that they had about 82% identity at the nucleotide level with the corresponding region of rice PSY cDNA. The three unique PSY cDNA sequences identified from wheat endosperm cDNA are shown in FIG. 11. Thirty-five single nucleotide polymorphisms (SNPs) were identified between the 3 PSY sequences and of these, 9 SNPs were unique to ‘psyform1’, 13 SNPs were unique to ‘psyform2’ and 13 SNPs were unique to ‘psyform3’. There were no nucleotide positions where all 3 sequences were polymorphic. The 3 PSY sequences appear to be from a homologous locus and are most similar to the rice chromosome 12 PSY gene (82% identity). Therefore, it was predicted the genes for wheat PSY homeoforms 1, 2 and 3 would be located on wheat chromosome 5S on the A, B and D genomes.

Specific primers (psyform 1f, 2f, 3f, 1r, 2r and 3r, Table 4) based on SNPs between the 3 wheat endosperm PSY homeoforms were designed and used to individually amplify the genomic fragment from wheat DNA corresponding to each homeoform. The forward homeoform specific primers had nucleotide sequences based on the wheat sequences corresponding to nucleotide positions 588-606 bp (relative to the rice cDNA sequence AY024351) and the reverse homeoform specific primers had sequences corresponding to the complement at positions 692-710 (FIG. 11).

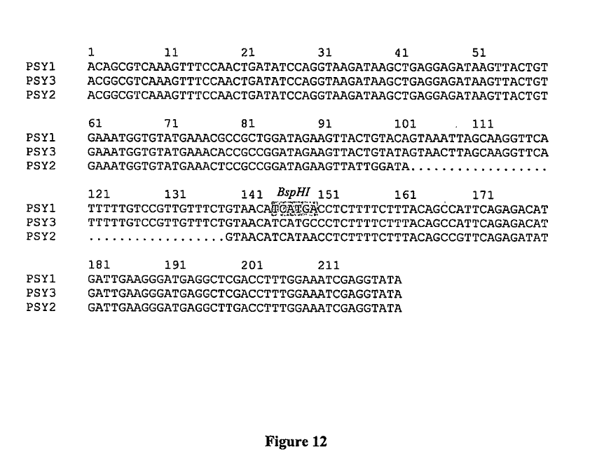

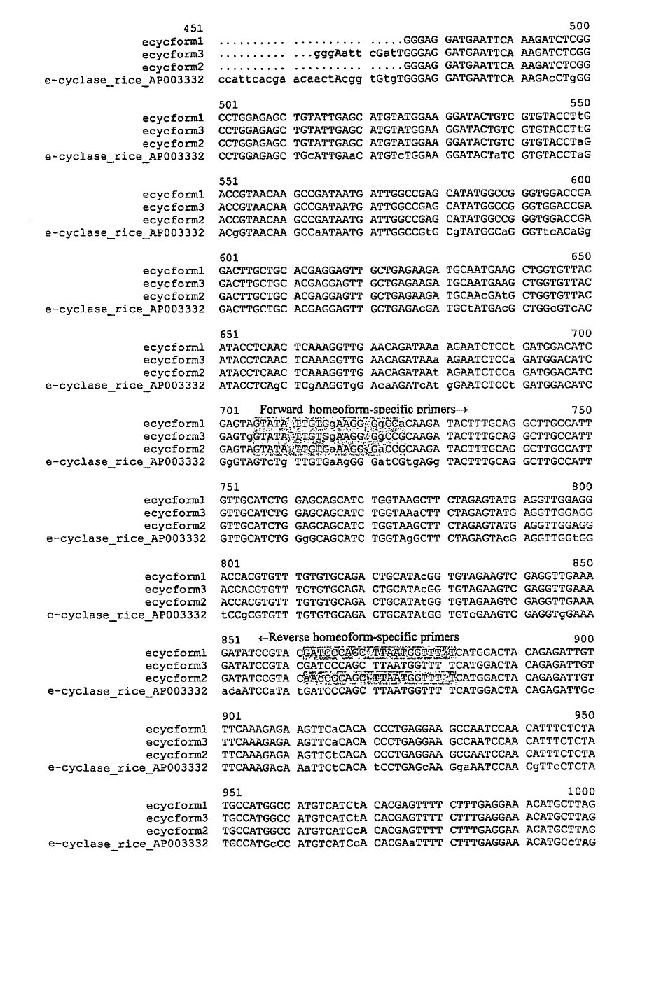

As shown by gel electrophoresis of the PCR reaction products, the products generated with ‘psyform1’ or ‘psyform3’ primer pairs were 275 bp long. When amplified with ‘psyform2’ primers, the product was 250 bp long, which could be explained by the presence of a deletion in the intron of ‘psyform2’ (FIG. 12). The PSY1 and PSY3 fragments could be distinguished by BspHI digestion of the PCR products due to the presence of the restriction site in the PSY1 fragment but not the PSY3 fragment. Amplification of genomic DNA with these homeoform specific primers and subsequent digestion of the PCR products with BspHI was therefore used to distinguish the three wheat PSY homeoforms.

Genomic DNA of Chinese Spring nullisomic/tetrasomic lines, characterised by an absence of one chromosome and duplication of homologous chromosomes, was then used in amplification reactions with each of the homeoform-specific primer pairs, focussing particularly on the chromosome 5 and chromosome 7 null lines. Amplification of each homeoform was successful for each of the 7A, 7B and 7D null lines, indicating the PSY1-3 homeoforms were not located on chromosome 7. The 1^{st} PSY homeoform was not amplified from the 5D nulli/tetra genomic DNA, indicating it was located on chromosome 5D. The 2^{nd} homeoform was not amplified from the 5A nulli/tetra genomic DNA, indicating it was located on 5A. The wheat genes corresponding to the isolated PSY1 and 2 homeoforms were therefore located on chromosome 5.

Despite utilising three primer sets designed to different SNP regions, the 3^{rd} PSY homeoform was amplified from all null lines tested, and could not be located to any specific chromosome. It is probable that a specific primer set could not be found for this PSY homeoform due cross-amplification with any of the 6 unidentified homeoforms from the other 2 PSY loci. It may also be possible that there are more PSY duplications in wheat than in rice. Wheat genes have undergone gene duplication more often than rice, with 25% of all wheat genes having undergone duplication (Akhunov et al., 2003).

Although nine PSY isoforms were possible in wheat, only three different cDNA sequences were isolated. The additional PSY isoforms might not be expressed in wheat endosperm in a similar way to maize where there are 2 PSY genes, one of which is not expressed in endosperm (Gallagher et al., 2004). In an attempt to identify further PSY sequences, RNA from leaves was isolated, reverse transcribed and used in PCR with the PSYFOR and PSYREV primers, used previously with the endosperm cDNA. Despite sequencing more than 20 individual clones containing leaf cDNA PSY sequences, no further isoforms of PSY were identified; rather all sequences were identified as PSY homeoforms 1 or 2. Therefore, the likely explanation for the lack of amplification of the other PSY isoforms from wheat was that the PSY primers were homologous only to the wheat PSY corresponding to the rice chromosome 12 PSY isoform.

### Example 7

### Wheat Lycopene ε-Cyclase Genes

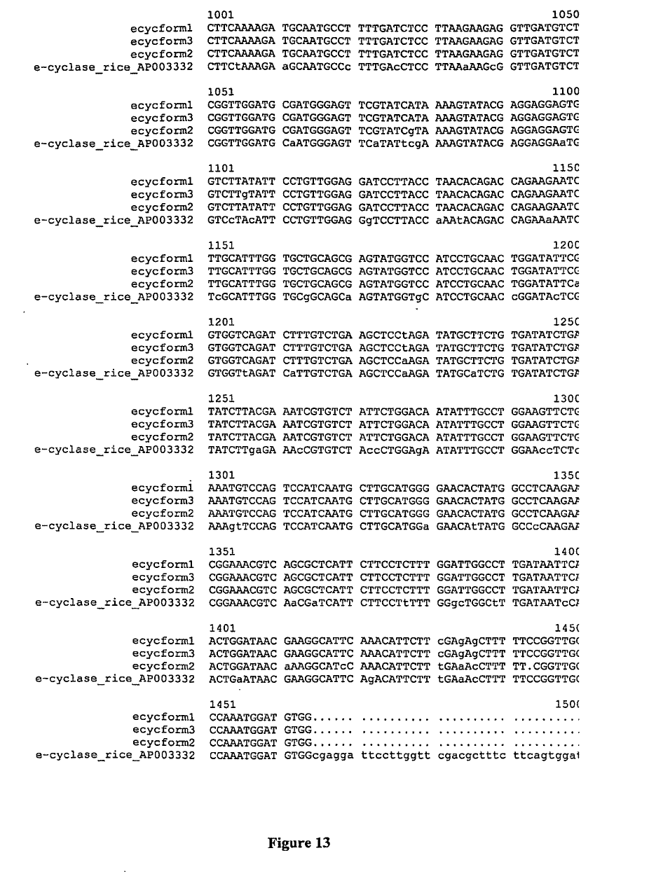

Rice has one ε-cyclase locus located on chromosome 1 and there was no evidence for other ε-cyclase genes in rice. Other plants also appear to have only one ε-cyclase gene including Arabidopsis and tomato. It was therefore predicted that there would be one corresponding locus in wheat, with homologous genes on A, B and D genomes, producing 3 homeoforms. A primer pair, epsilonf and epsilonr (Table 4), was designed to conserved regions of genes encoding or possibly encoding ε-cyclase in lettuce (AF321538), marigold (AY099485), Arabidopsis (AY079371), spinach (AF463-497), citrus (AF486650), rice (AP003332) and tomato (X86452). PCR reactions using this primer pair and wheat endosperm cDNA generated 990 bp PCR products. These were cloned into pGEM-T and 20 clones sequenced. Analysis of the sequences showed two distinct sequences each with about 86% identity to the corresponding region of the rice ε-cyclase nucleotide sequence. The wheat endosperm sequences were designated ‘ecycform1’ and ‘ecycform2’ (FIG. 13).

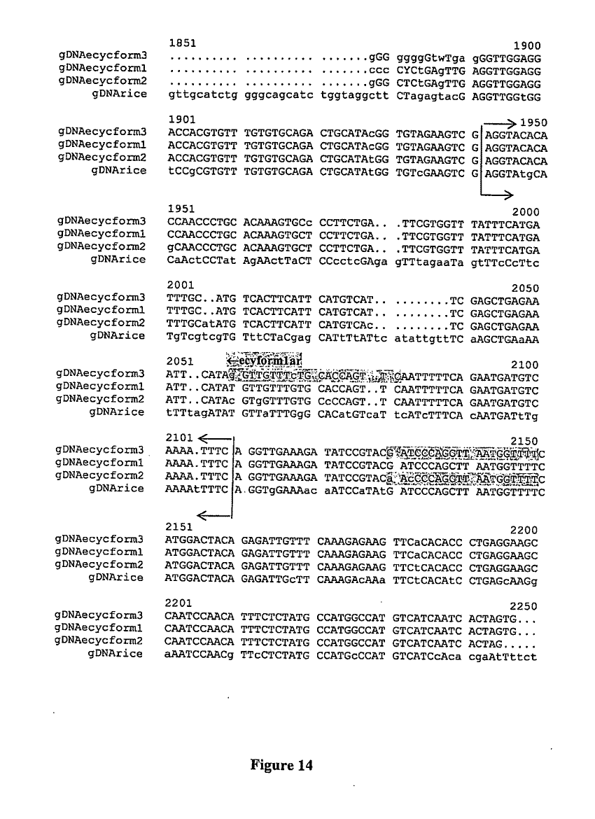

To isolate the predicted third wheat homeoform, two new primers were designed to amplify across a 350 bp section of genomic DNA that was predicted to contain an intron. As introns are often more polymorphic than exons, this was thought to increase the likelihood of identifying variant sequences. ECYCF/INTRON/BsiWI was used in conjunction with ECYCR/BsiWI (Table 4) to amplify the 350 bp genomic DNA fragment, which was cloned into pGEM-T and 20 clones sequenced. A third genomic sequence was indeed identified, containing an intron, which on the basis of SNPs identified this fragment as the third homeoform of ε-cyclase (FIG. 14). Comparison of the genomic sequences with the ε-cyclase cDNA sequences from wheat confirmed that the third homeoform sequence was present in the endosperm cDNA. It was possible that this sequence was not identified in the initial screen because of the close similarity with the homeoform ecycform1 (FIG. 13) or because of less representation in the cDNA. The 3^{rd} homeoform sequence was only represented once in the 20 individual clones sequenced for the ε-cyclase fragment. However, this did confirm that all three ε-cyclase homeoforms were expressed in the endospern of wheat.

There were 24 SNPs identified between the 3 ε-cyclase sequences and of these, 3 SNPs were unique to ‘ecycform1’, 19 SNPs were unique to ‘ecycform2’ and 4 SNPs were unique to ‘ecycform3’ (FIG. 13).

To amplify each wheat homeoform individually from genomic DNA, forward primers (ecycform1F, 2F and 3F, Table 4) were designed based on the cDNA sequence SNPs. The forward primers corresponded in sequence to nucleotide positions 706-725 (relative to the rice cDNA sequence, AP003332) which region contained 3 SNPs, 2 of which were in the last 4 bp of the primers (FIG. 13). The reverse primers (ecycform1R, 2R) used to amplify homeoforms 1 and 2 were designed over cDNA SNPs, and corresponded to the complement of the sequences at position 862-881 in the rice cDNA sequence (FIG. 13). The reverse primer used to amplify homeoform 3 (ecycform1ar) was designed over an intron SNP, identified from the genomic sequences (FIG. 14) due to the lack of unique cDNA SNPs for this sequence.

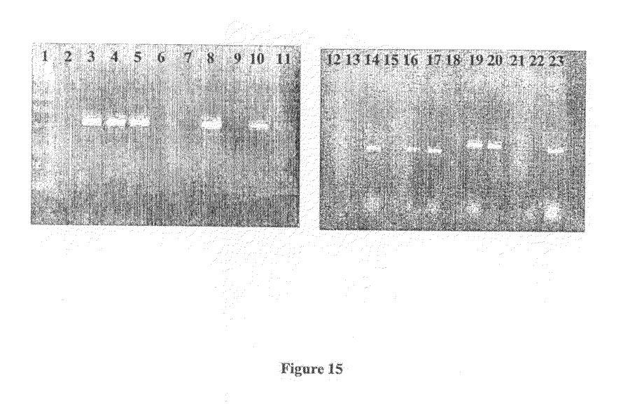

Amplifications using these SNP-specific primer pairs and genomic DNA from the nullisomic/tetrasomic and diteleo (in which whole chromosome arms are deleted) Chinese Spring lines, focussing on lines that lacked all or part of chromosome 3A, 3B or 3D. Gel electrophoresis of the products (FIG. 15) showed that the 1^{st} ε-cyclase homeoform was located on chromosome 3D, the 2^{nd} homeoform was located on 3A and the 3^{rd} homeoform was located on 3B (FIG. 15).

Further mapping with bin deletion lines suggest that the genes for epsilon cyclase are located close to the centromere.

### Example 8

### Genomic Sequences of Wheat ε-Cyclase Homeogenes

To identify polymorphisms between the ε-cyclase genes on chromosome 3B of the cultivars Sunco and Tasman and so confirm that the ε-cyclase gene underlay the observed QTL on chromosome 3B, genomic sequences for the genes from 3B were obtained. As a preliminary step, the genomic sequences for all three homeoforms (3A, 3B and 3D) were first obtained from a reference cultivar. Chinese Spring was chosen as the reference cultivar as the initial cDNA sequences were from this cultivar and many deletion lines are available for mapping in this background.

To complete the genomic sequence, genome walking was performed using libraries of Chinese Spring DNA cleaved with specific restriction enzymes and a Universal Genome Walker Kit (BD Biosciences Clontech Palo Alto US) as per the manufacturer's instructions. Two walks in the downstream and one walk in the upstream direction were performed; the primers used are given in Table 4.

In order to amplify the 3′ ε-cyclase sequence, two gene specific forward primers, walk1f1 and walk1f2 were used in two rounds of PCR with the two adapter-specific primers, AP1 and AP2, using each of the libraries as template in 4 separate reactions. An approximately 500 bp fragment obtained from library number 2 (EcoRV digested gDNA) was sequenced and showed 76% identity to rice ε-cyclase 3′. The rice 6-cyclase sequence was 3480 bp long and the homology began at about position 3050 and continued 20 bp past the end of the rice ε-cyclase gDNA sequence.

The Chinese Spring sequence obtained for the 3′ end of ε-cyclase was subsequently used to design two more forward primers, walk2f1 and walk2f2 which were used in conjunction with the adapter primers to obtain sequence further downstream. A 1311 bp fragment obtained from library 3 (PvuII digested gDNA) was sequenced and BLAST analysis showed a 62% identity of the sequence to a rice threonine kinase1-like gene. It was concluded that the wheat sequence spanned the short intragenic region between ε-cyclase and the downstream gene, before showing homology to the downstream gene, the threonine kinase1-like gene.

In order to amplify the 5′ ε-cyclase sequence, two gene specific reverse primers, walk1r1 and walk1r2 were used in two rounds of PCR with the two adapter-specific primers, AP1 and AP2. The 450 bp fragment obtained from library 4 (StuI digested gDNA) was sequenced and showed 67% identity to the corresponding rice ε-cyclase genomic sequence. The homology began at about position 1550 bp and continued to ˜1110 bp of rice ε-cyclase genomic sequence. Subsequent walks to identify exon1 and intron1 were unsuccessful, thus an alternate strategy was used.

A barley EST (TIGR ID TC96054) that contains exons 1 and 2 of ε-cyclase, identified on the basis of homology to the rice genomic sequence, was used to design a primer 5′ EC Ex1a (TAATCGCCGTCTGCCACGTGCC) (SEQ ID NO:64) and used with walk2r1 to amplify exon and intron 1 from wheat.

After each PCR reaction was completed, the products were cloned and sequenced. Using the sequences obtained, further primers were designed and the region re-amplified and cloned. Twenty clones were sequenced and three distinct sequences identified after assembly, representing the three 6-cyclase genomic homeoforms. The complete genomic sequences for the A, B and D homeoforms of ε-cyclasefrom wheat provided as SEQ ID NO's 16 to 18 respectively. The nucleotide sequences of the predicted coding regions are provided as SEQ ID NO's 19 to 21 respectively. Furthermore, predicted amino acid sequence of each isoform is provided as SEQ ID NO's 22 to 24 respectively.

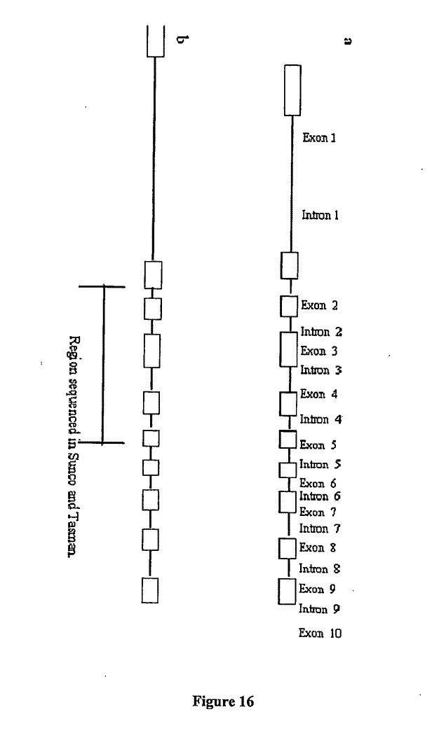

The exon/intron structure of ε-cyclase from a) rice and b) wheat is provided in FIG. 16.

The nucleotide sequences of the coding regions had significant homology with the available barley and rice sequences at both the nucleotide and amino acid levels (Tables 5 and 6). The greatest homology was to barley, then rice, and then Arabidopsis.

### Example 9

### Fine Mapping of the Wheat ε-Cyclase Gene

A SNP or sequence difference between the Sunco 3B ε-cyclase and Tasman 3B ε-cyclase genes was sought to confirm that the 3B ε-cyclase gene was responsible for the observed QTL on chromosome 3B in the Sunco x Tasman doubled haploid population. As there was little genetic difference between Sunco and Tasman, a large region encompassing the gene including introns and the 5′ and 3′ flanking regions were sequenced in order to find polymorphisms. The amount of ε-cyclase sequence available after genome walking totaled about 4.6 kb. The exon/intron structure of wheat ε-cyclase showed conservation with rice ε-cyclase, having 10 exons and 9 introns. In order to identify SNPs between parent cultivars, Sunco and Tasman, PCR primer sets were designed over the length of available sequence.

TABLE 5
Percent identity of the nucleotide sequences of the predicted coding
regions of ε-cyclase cDNAs. Percent identity was calculated after
a local sequence alignment was performed using the GAP program,
with default settings, in the Wisconsin GCG package (Deveraux et al.,
1984). The barley sequence was only a partial sequence and was an
assembly of ESTs from the TIGR barley EST database
(http://www.tigr.org/tigr-scripts/tgi/T_index.cgi?species=barley).
A, B and D represent the three wheat homeoforms.

| | Arabidopsis | Barley | Rice | | |
|---|---|---|---|---|---|
| | At5g57030 | TC92298 | XM_463351 | A | D |
| Barley | 69 | | | | |
| TC92298 | | | | | |
| Rice | 66 | 89 | | | |
| XM_463351 | | | | | |
| A | 64 | 97 | 85 | | |
| D | 65 | 97 | 85 | 98 | |
| B | 65 | 97 | 84 | 98 | 99 |

TABLE 6
Percent identity of amino acid sequences of the predicted ε-cyclase
proteins. Percent identity was calculated after a local sequence
alignment was performed using the GAP program, with default
settings, in the Wisconsin GCG package (Deveraux et al., 1984).
Annotation is as per Table 5.

| | Arabidopsis | Barley | Rice | | |
|---|---|---|---|---|---|
| | At5g57030 | TC92298 | XM_463351 | A | D |
| Barley | 72 | | | | |
| TC92298 | | | | | |
| Rice | 78 | 94 | | | |
| XM_463351 | | | | | |
| A | 71 | 98 | 86 | | |
| D | 72 | 98 | 87 | 97 | |
| B | 72 | 98 | 87 | 97 | 99 |

One region was amplified using the forward primer epsilonf in conjunction with either ecycform1R or RTECYCR. epsilonf was used in PCR with eycform1R, which could bind to homeoforms 1 and 3, to amplify a 750 bp gDNA fragment from both Sunco and Tasman. The 750 bp PCR product was cloned and individual colonies sequenced. Comparison of Sunco and Tasman fragments for this 750 bp region showed no sequence differences for the B homeoform. A slightly longer fragment was PCR amplified for this region to obtain an extra 300 bp of sequence. epsilonf was used in conjunction with RTECYCR. The RTECYCR bound 300 bp downstream from eycform1R, amplifying a 1 kb gDNA fragment from both Sunco and Tasman when used in PCR with epsilonf Once again, fragments from Sunco and Tasman were cloned and sequenced and it was determined Sunco and Tasman D homeoforms were identical for the 1 kb region.

A further region amplified was toward the furthermost end of the ε-cyclase sequence. IntraF was used in conjunction with IntraR. These primers bound on either side of the intragenic region between 3′ ε-cyclase and downstream threonine kinase. The PCR was carried out with the presumption that all 3 homeoforms would be amplified from both Sunco and Tasman, as only one gDNA sequence was available for this region. The 880 bp fragments from both Sunco and Tasman were cloned and sequenced. All three homeoforms were identified from both cultivars and only one SNP that specifically distinguished the 3B homeoforms of Sunco and Tasman from each other was identified. This can be used to fine map the 3B homeoform of ε-cyclase within the Sunco x Tasman population.

As a second strategy to map the ε-cyclase gene on chromosome 3 more finely, the deletion lines of Endo and Gill (1996) will be used to show that ε-cyclase locates to the same deletion bin as the QTL. The marker gwm285 which is closest to the QTL identified on 3B has been shown to map to bin 3BS1-0.33 (http://wheat.pw.usda.gov/cgi-bin/graingenes/report.cgi?class=breakpointinterval;name=C-3BS1-0.33:show=locus). However, in the wheat SSR 2004 consensus map gwm285 isd located close to the centromere on the long arm of chromosome 3B (http://www.gramene.org/db/cmap/map_details?ref map_set_aid=sod2004a&ref map_aids=sod2004a3b). The primers specific for the 3B homeoform of ε-cyclase and those from gwm285 can be used to determine whether they are located within the same deletion bin. If this is the case, this will provide strong evidence that ε-cyclase co-locates to the same region of the chromosome as the QTL.

### Discussion

Based on the above experiments, the wheat 3B ε-cyclase gene was a prime candidate gene responsible for the QTL for lutein content observed on chromosome 3B. Once a suitable polymorphism has been identified between Sunco and Tasman, it can be used as a molecular marker to confirm that the gene for ε-cyclase locates to the region of the QTL.

Parker and Langridge (2000) developed an STS marker linked to flour colour on chromosome 7A, using the Schomburgk x Yarralinka population, which was later validated by Sharp et al., (2001) in other cultivars, including the Sunco x Tasman doubled haploid population used in this study. The STS markers were considered diagnostic of the yellow flour allele across a range of cultivars. Genetic characterisation of the 7A QTL in wheat was incomplete but in other species the role of homologous chromosomes in colour determination has been confirmed. Chromosomes from wild type barley, H. chilense, were added to bread wheat and chromosome 7H^{ch}, which is a homologue to chromosome 7 in wheat, conferred increased yellow pigment to bread wheat (Alvarez et al., 1998). A combination of the 7A STS marker and a marker for the gene underlying the QTL on 3B could prove a strong predictive tool for flour colour.

Francki et al (2004) created a rice-wheat synteny chart based on known mapped wheat ESTs and also attempted to identify genes in QTL regions controlling carotenoid content. Wheat ESTs mapped to bin locations on 3S and 7L were searched for orthologs in rice. The rice orthologs were then aligned to wheat chromosomes 3S and 7L. Rice orthologs were used to identify contiguous rice BAC clones for chromosomal alignments. Overlapping contiguous rice BAC clones that were anchored to similar regions of wheat chromosomes were defined as a macrosyntenic unit. All mapped wheat ESTs were then aligned to the macrosyntenic units to validate synteny or identify breaks in synteny. The power of synteny mapping in this method, depended on a comprehensive number of mapped wheat ESTs to identify rice orthologs, assigned to BACs, which are aligned to wheat chromosomes. Before identifying the wheat ε-cyclase encoding sequences disclosed herein the inventors were unable to find a wheat EST available for ε-cyclase, hence the corresponding ortholog in rice was not identified. Furthermore, no wheat ESTs with corresponding orthologs from the BAC clone AP003332 containing ε-cyclase were identified and so this BAC clone did not appear on their synteny map. For this reason, ε-cyclase was not identified by Francki et al., in the QTL region of wheat. However, after performing the experiments described herein an EST was identified (Accession No. BQ240373) which is about 140 nucleotides in length and encodes about the first 24 amino acids of a wheat ε-cyclase, however, the nature of the sequence was not suggested in the EST annotation.

Rather, analysis of rice sequences aligned to the region of the 3BS QTL identified two types of putative prenyltransferases of interest (Francki et al., 2004). The three classes of prenyltransferases serve a variety of biological functions (Liang et al., 2002). One class of prenyltransferases are the isopentenyl pyrophosphate synthases (IPPSs), of the isoprenoid pathway which catalyse the addition of isopentenyl pyrophosphate (IPP) units onto other allylic pyrophosphate substrates, giving rise to GGPP (Britton, 1998). IPP and GGPP are essential building blocks of carotenoid biosynthesis (Cuttriss and Pogson, 2004). Francki et al., (2004) proposed that prenyltransferases might limit the availability of GGPP for carotenoid biosynthesis, thereby regulating flour colour and underlying the 3BS QTL. However, the 2 prenyltransferases identified in the 3BS region were actually from another the class of prenyltransferases known as protein prenyltransferases. Protein prenylation in plants occurs in a similar pattern to that observed in mammalian cells, whereby prenyltransferases catalyse the transfer of farnesyl C(15) and geranylgeranyl C(20) groups to cysteine residues of proteins. A type of prenylation confined to chloroplasts also occurs in plants yet little is known of this non-conventional prenylation (Parmryd et al., 1999). Prenylated proteins are involved in specific signal-transduction pathways, and prenylation of proteins is often required for protein function (Liang et al., 2002). A relationship between protein prenylation and carotenoid synthesis was not established and it seems to us much less likely that protein prenyltransferases underlie the 3B QTL region than ε-cyclase.

### Example 10

### Expression of Wheat ε-Cyclase in Escherichia coli

The wheat genes identified above had very high homology to other known ε-cyclases making it likely they encoded ε-cyclases. One way to prove functionality would be to perform experiments similar to those described by Cunningham et al., (1996) using a bacterial expression system. The coding regions of the A, B and D homeoforms of ε-cyclase from wheat will be cloned in frarne into the vector pBluescript SK+. The plasmids obtained will be used to transform an E. coli strain which contains the plasmid pAC-LYC. This strain accumulates the carotenoid lycopene because of the genes present on pAC-LYC, resulting in the colonies being a pink colour. The transformed colonies with both plasmids would be expected to have a deep yellow colour as the result of expression of ε-cyclase and conversion of the lycopene to 6-carotene, similar to the result seen when ε-cyclase from Arabidopsis was expressed in this cell strain. Analysis of the carotenoid profile of the resulting cell strains will be determined by HPLC to confirm the new compounds identity.

### Example 11

### Construction of RNAi Vectors and Transformation of Wheat

In order to reduce ε-cyclase activity in wheat, constructs encoding an RNA molecule that inhibits the endogenous ε-cyclase gene expression could be used. For example, RNAi (hairpin RNA) constructs that express a double-stranded RNA molecule including at least part of the ε-cyclase (sense) RNA sequence and the corresponding complementary (antisense) strand could be used. Such RNAi molecules have been shown to efficiently reduce gene expression in plants.

To demonstrate this approach for ε-cyclase in wheat, two duplex constructs were assembled each containing a region of the wheat ε-cyclase cDNA sequence inserted in both sense and antisense orientations. The first construct used the pStarling vector, containing the constitutive ubiquitin promoter, and therefore designed for the inhibition of ε-cyclase in all tissues of the plant. The second construct contained the grain or endosperm-specific promoter, pBx17, and was designed for the reduction of ε-cyclase activity in the grain only.

RNAi Construct with Ubiquitin Promoter

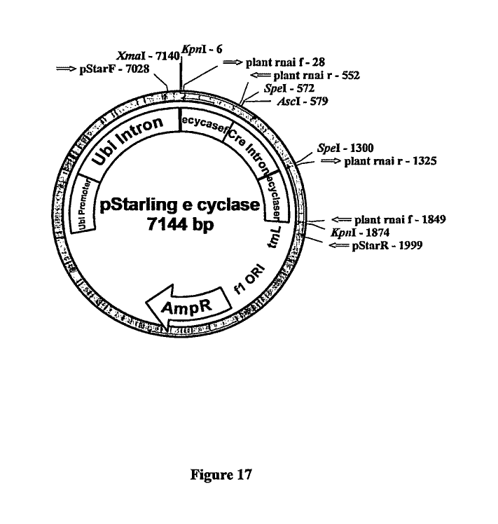

The primers plantrnaif (5′ CCCGGGGGTACCAATGATTGGCCGAGCATATGG 3′ which contains KpnI and XmaI restriction sites) (SEQ ID NO:65) and plantrnair (5′ GGCGCGCCACTAGTGGTAAGGATCCTCCAACAGG 3′ (SEQ ID NO:66) which contained introduced SpeI and AscI restriction sites) were used to amplify a 588 bp fragment from an ε-cyclase cDNA clone. Using the restriction sites introduced by the primers, the PCR product was cloned into the vector pStarling in both the sense (XmaI and AscI) and antisense (KpnI and SpeI) orientations to produce the vector pStarling/ε-cyclase (FIG. 17). Restriction mapping and sequencing were performed to ensure the plasmid was correct prior to use for wheat transformation.

### Grain-Specific RNAi Construct

The primers grainrnaif (5′ CTCGAGGCTAGCAATGATTGGCCGAGCATATGG 3′ which contained NheI and XhoI restriction sites) (SEQ ID NO:67) and grainrnair (5′ GAATTCACTAGTGGTAAGGATCCTCCAACAGG 3′ (SEQ ID NO:68) which contained SpeI and EcoRI restriction sites) were used to amplify the same 588 bp fragment from an ε-cyclase cDNA clone used in the whole plant construct. Using the restriction sites introduced by the primers, the PCR product was cloned into the vector pZLRint9 in both the sense and antisense orientations to produce the vector pZLRint9/ε-cyclase. The ε-cyclase fragments and intron sequence were subcloned into the pBx17CasNKEco vector, containing the grain-specific high molecular weight glutenin Bx17 promoter to produce the vector pBx17Ecyc (FIG. 18). The sequence of pBx 17 E cyc was confirmed by sequencing.

The coding sequence of the RNAi construct is provided in SEQ ID NO:25. The sense exon is nucleotides 1 to 564 of SEQ ID NO:25, an intron sequence which forms the loop structure is encoded by nucleotides 565 to 1075, whereas the antisense exon which when transcribed will form dsRNA with the transcribed sense exon is nucleotides 1076 to 1640.

### Wheat Transformation and Analysis of Transgenic Plants

Transformation of wheat embryos from the cultivar Bobwhite 26 was performed according to the method of Pellegrineschi et al. (2002). To confirm that the plants that were produced contained the construct, PCR analysis was performed on genomic DNA extracted from leaves as described in Example 1.

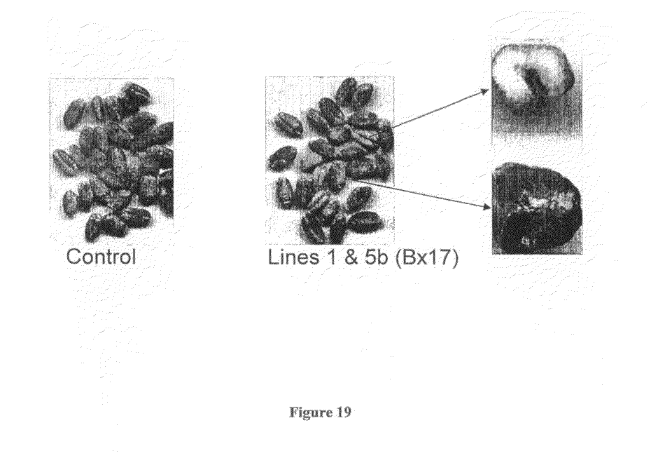

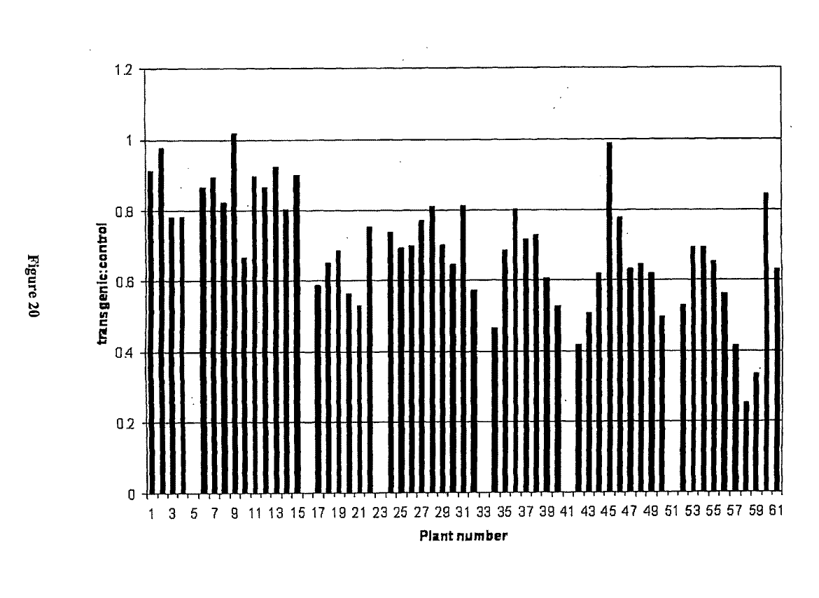

Seven transformed wheat lines that contained the grain specific construct pBx17Ecyc were identified and 12 that contained the whole plant construct pStarling/ε-cyclasewere identified. In two of the lines that contained the grain specific construct, a proportion of the mature T1 seeds had significantly lighter endosperm than the control plants (FIG. 19), indicating a reduction in the level of lutein and therefore a reduction in ε-cyclase activity in the endosperm. T1 seeds from several of these lines were sown in soil and grown in the greenhouse to maturity, and T2 seeds harvested. Flour samples from the grain were analysed for carotenoid content by HPLC as described in Example 1. The data for lutein content (FIG. 20) showed that most lines exhibited reduced lutein content compared to the wild-type control (cv. Bobwhite), while some lines were reduced by at least 70%. These lines were predicted to be substantially unaltered in their lutein content in tissues other than seed.

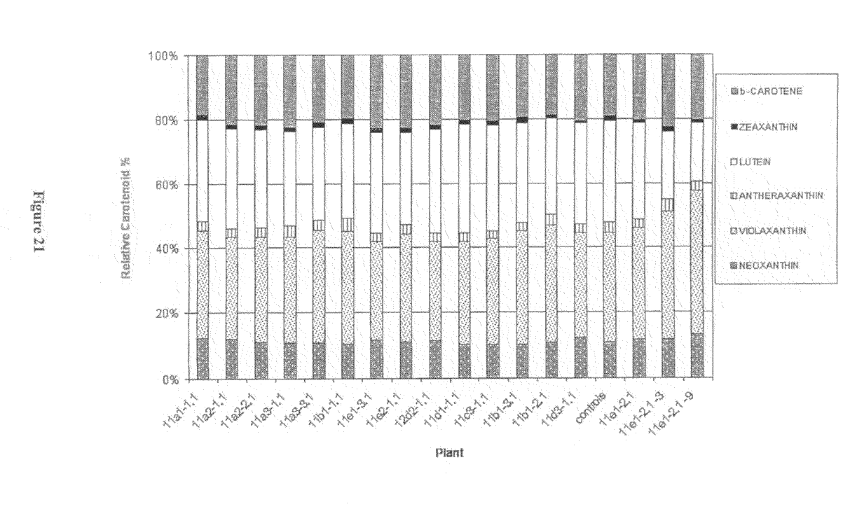

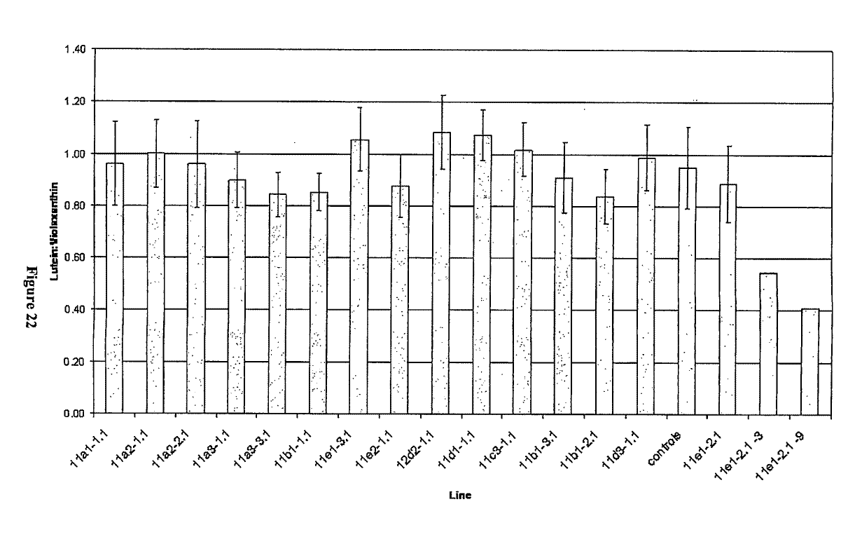

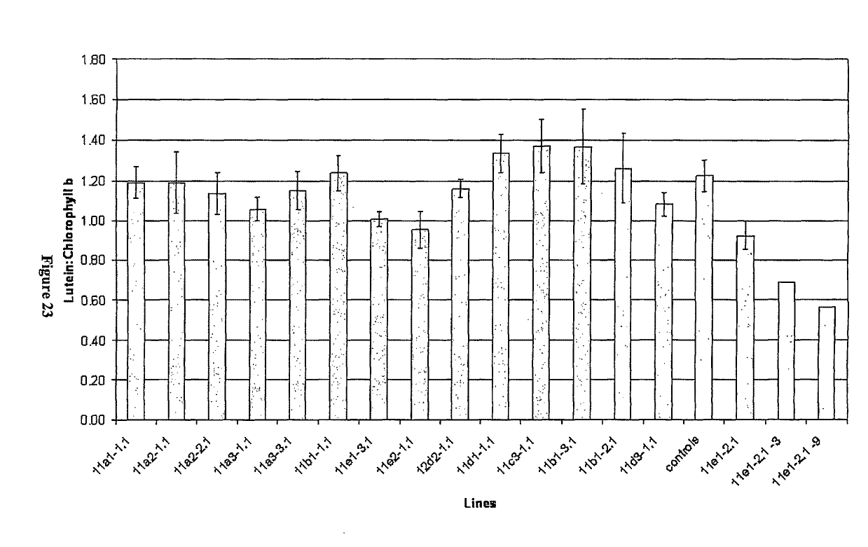

Analysis of the carotenoid composition of leaves from T0 wheat plants containing the whole plant construct pStarling/ε-cyclase indicated that in some transformed lines the lutein:violaxanthin ratio was reduced by up to 50%. Some lines showed an accumulation of small quantities of antheraxanthin. In Arabidopsis, antheraxanthin is thought to accumulate only in lines which are unable to synthesise lutein. T1 progeny plants were also examined for carotenoid composition in the leaves. Total carotenoids were extracted from 10 plants from each of 15 trangenic lines and analysed by HPLC. Only two plants from one line showed a significant decrease in the lutein content (FIG. 21). In plant 3 from line 11e1-2.1, the lutein content was decreased by 32% compared to the control, while in plant 9 from the same line, lutein was decreased by 43%. In the same two plants, violaxanthin content increased by 15% and 40%, respectively. When the data was expressed as a ratio of lutein:violoaxanthin (FIG. 22), the ratio was 0.55 in plant No. 3 and 0.41 in plant No. 9, compared to the control (0.95±0.16), representing decreases of 42% and 47%, respectively. When the lutein content was compared to the chlorophyll b content and expressed as a ratio of lutein:chlorophyll b, the decreases were 25% and 39% respectively (FIG. 23). These data show that lutein content in tissues other than seed can also be decreased, although fewer down-regulated lines were obtained. This also indicated that tissue-specific gene silencing is preferred over whole-plant silencing.

These experiments confirmed that the isolated genes could indeed be used to modify ε-cyclase activity in wheat and reduce lutein levels. Interestingly and surprisingly, this was associated with an apparent increase in levels of at least one compound in the β-carotene branch of the pathway, which were concluded to be a consequence of the reduced activity in the ε-carotene branch. Moreover, the block in the α-carotene specific branch of the pathway through ε-cyclase would be expected to have advantages over a block in an earlier step (such as PDS or PSY) common to both α- and β-carotene branches, which would be expected to reduce the levels of ABA and other desired compounds. The use of the ε-cyclase gene sequences is therefore advantageous in wheat.

### Example 12

### Screening for Naturally Occurring Null Mutations of Epsilon Cyclase

In order to create non-transgenic lines that are null for epsilon cyclase it is necessary to identify lines that contain null mutations in each of the three genomes and then cross these to produce a line that is null in all three genomes. To identify lines that have null mutations in individual genomes Ecotilling (Comai et al., 2004) will be used.

For each homeoform genome specific primers, that are asymmetrically labelled with floursecent tags, will be developed and used for amplification of the targeted genomic region. Genomic DNA from the reference cultivar (Chinese Spring) and each of the cultivars to be screened will be mixed in a 1:1 ratio, and used as the template for each amplification reaction. After the amplification reactions are complete the products will be subjected to an extension, denaturation and reannealing process (72° C. for 8 min, 98° C. for 8 min, 80° C. for 20 s followed by 60 cycles of 80° C. for 7 s decreasing 0.3° C. per cycle).

The annealed products will then be digested with 30 μl CelI enzyme (prepared as described in Colbert et al., 2001) at 1 μl CelI/300 μl buffer containing 10 mM HEPES, pH 7, 10 mM KCl, 10 mM MgCl2, 0.002% Triton X-100 and 10 μg/ml BSA. Samples were precipitated with 10 μl of 2.5 M NaCl with 75 mM EDTA, pH 8.0, and 80 μl of isopropanol, centrifuged for 30 min at 3,220 r.p.m. and resuspended in 8 μl of formamide loading buffer (33% deionized formamide, 10-50 mM Tris, pH 7.5, 1 mM EDTA and ˜0.02% bromphenol blue), then heated at 80° C. for 7 min, denatured at 95° C. for 2 min and placed on ice (Slade et al., 2005).

Samples will then be analysed as described in Slade et al., (2005). Digested PCR products will be electrophoresed through a 6.5% polyacrylamide, 7 M urea gel in 0.8×TBE running buffer at 1,500 V, 40 mA, and 40 V settings on a LI-COR2 gel analyzer (LI-COR). Images will be analyzed visually for the presence of cleavage products using Adobe Photoshop software (Adobe Systems Inc.). The presence of cleavage products indicates that the line being screened contains SNPs when compared to the reference cultivar. The PCR product from those lines in which SNPs have been identified will be sequenced and the nature of the SNP determined.

Using this technique we will be able to rapidly identify SNPs that result in frameshift or stop mutations in each of the three homeoforms. These lines can then be used to create double and triple null lines of epsilon cyclase using standard crossing-techniques.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

AACC (2000) Determination of Pigments, Method 14-50. In Approved methods of the AACC, Ed 10. The Association, St. Paul, Minn.
Akhunov et al., (2003) Genome Research 13:753-763
Almeida and Allshire (2005) TRENDS Cell Biol 15: 251-258
Alvarez et al., (1998) Plant Breeding 117:287-289
Alvarez et al., (1999) Plant Breeding 118:187-189
Alvarez et al., (1994) Cereal Chem 71:517-519
Bartley and Scolnik (1995) Plant Cell 7:1027-103
Beyer et al., (2002) J Nutrition 132:506 S-510S
Bourque (1995) Plant Sci. 105:125-149
Bouvier et al., (2000) Eur J Biochem 267:6346-6352
Brandt et al., (1985) Carlsberg Res. Commun. 50:333-345
Britton (1998) In Britton et al., eds, Carotenoids, Vol 3. Birkhauser, Base1, pp 13-147
Buckner et al., (1996) Genetics 143:479-488
Burkhardt et al., (1997) Plant J 11:1071-1078
Calucci et al., (2004) J Agric Food Chem 52:4274-4281
Cenci et al., A (2004) Genome 47:911-917
Colbert et al. (2001) Plant Physiol. 126:480-484
Colot et al., (1987) EMBO J. 6: 3559-3564
Comai et al., (2004) Plant J. 37:778-786
Cramer et al., (2001) Int J Cancer 94:128-134
Cunningham and Gantt (1998) Ann. Rev. Plant Physiol Plant Mol. Biol. 49:557-583
Cunningham and Gantt (2001) Proc Natl Acad Sci USA 98:2905-291
Cunningham et al., (1993) Febs Letts 328:130-138
Cunningham et al., (1994) Plant Cell 6:1107-1121
Cunningham et al., (1996) Plant Cell 8:1627-1639
Cuttriss et al., (2005) In K Hoober, ed, The Structure and Function of Plastids: Advances in Photosynthesis and Respiration. Kluwer, In Press
Cuttriss and Pogson (2004) In K Davies, ed, Plant Pigments and Their Manipulation.
Blackwell Publishing, Oxford UK, pp 57-82
Demmig-Adams and Adams (1996) Trends Plant Sci. 1:21-26
Devereux et al., (1984) Nucl Acids Res 12:387-395
Eagles et al., (2001) Australian Journal of Agricultural Research 52:1349-1356
Elouafi et al., (2001) Hereditas 135:255-261
Endo and Gill (1996) J Hered 87:295-307
Feillet et al., (2000) J. Cereal Sci. 32:215-233
Francki et al., (2004) Funct. Integr. Genomics 4:118-130
Gale and Devos (1998) Proc Natl Acad Sci USA 95:1971-1974
Gallagher et al., (2004) Plant Physiol 135:1776-1783
Gilmore and Yamamoto (1991) Plant Physiol 96:635-643
Giuliano et al., (1993) Plant Cell 5:379-387
Good (2002) Cereal Foods World 47:5-6
Harayama (1998) Trends Biotechnol 16:76-82.
Harker and Hirschberg (1998) In Photosynthesis: Molecular Biology of Energy Capture, Vol 297, pp 244-263
Haseloff and Gerlach (1988) Nature 334:585-591
Henikoff et al, (2004) Plant Physiol 135: 630-636
Hentschel et al., (2002) J Agric Food Chem 50:6663-6668
Hirschberg (2001) Curr Opin Plant Biol 4:210-218
Hornero-Mendez and Minguez-Mosquera (2000) J Agric Food Chem 48:1617-1622
Janick-Buckner et al., (1999) J Heredity 90:507-513
Johnston et al., (1980) Cereal Chem 57:447-448
Joppa and Williams (1988) Genome 30:222-228
Jukanti et al. (2004) Cereal Chem. 81, 481-485
Kaneko et al., (1995) Biosci Biotechnol Biochem 59:1-4
King (1976) Planta 132:43-51
Kirk and Tilney-Bassett (1978) In The Plastids: Their Chemistry, structure, growth and inheritance. Elsevier/North Holland Biomedical Press, Amsterdam, The Netherlands, pp 217-239
Landrum and Bone (2001) Archiv Biochem Biophys 385:28-40
Langridge et al., (2001) Aust J Agric Res 52:1043-1077
Lemieux (2000) Current Genomics 1:301-311
Lepage and Sims (1968) Cereal Chem 45:600-604
Li et al., (1996) Plant Mol Biol 30:269-279
Li et al., (2004). Functional Integrat Genomics 4:231-240.
Liang et al., (2002) Eur J Biochem 269:3339-3354
Lindgren et al., (2003) Plant Physiol 132:779-785
Maluf et al., (1997) J Exper Bot 48:1259-1268
Mann et al., (2000) Nature Biotechnol 18:888-892
Mares and Campbell (2001) Aust. J. Agric. Res. 52:1297-1309
Mares et al., (1997) In 47th Australian Cereal Chemistry Conference
Matthews et al., (2003) J Exper Bot 54:2215-2230
Matus et al., (1993) Acta Pharm Hung 63:247-256
Matus-Cadiz et al., (2003) Crop Sci. 43:219-226
McGraw et al., (2001) Physiol Biochem Zool 74:843-852
Millar and Waterhouse (2005) Funct Integr Genomics 5: 129-135
Moehs et al., (2001) Plant Mol Biol 45:281-293
Moss (1967) Aust J Exper Agric Animal Husbandry 7:463-464
Nachit et al., (1995) In Proc. Seminar on durum wheat quality in the Mediterranean regions, Vol 22, Zargoza ICARDA/CIHEAM/CIMMYT, pp 181-188
Needleman and Wunsch (1970) J. Mol. Biol. 48:443-453
Panfili et al., (2004) J Agric Food Chem 52:6373-6377
Park et al., (2002) Plant Cell 14:321-332
Parker et al., (1998) Theor. Appl. Genet. 97:238-245
Parker and Langridge (2000) Mol. Breed. 6:169-174
Parmryd et al., (1999) PNAS 96:10074-10079
Pasquinelli et al., (2005) Curr Opin Genet Develop 15: 200-205
Pellegrineschi et al., (2002) Genome 45:421-430
Perriman et al., (1992) Gene 113:157-163
Pfundel and Bilger (1994) Photosynth Res 42:89-109
Phillip and Young (1995) Photosynth. Res. 43:273-282
Piccaglia et al., (1998) Industrial Crops and Products 8:45-51
Pinzino et al., (1999) J Agric Food Chem 47:1333-1339
Pogson et al., (1996) Plant Physiol 111:109-109
Rishi et al., (2004) J Biotechnol 111:9-15
Rogozhin et al., (2001) Biol Bull 28:126-133
Ronen et al., (2000) Proc Natl Acad Sci USA 97:11102-11107
Santra et al., (2003) Cereal Chem 80:130-131
Schwartz et al., (1997) Science 276:1872-1874
Senior (1998) Biotech. Genet. Engin. Revs. 15:79-119
Seo and Koshiba (2002) Trends Plant Sci 7:41-48
Sharp et al., (2001) Aust J Agric Res 52:1357-1366
Shewmaker et al., (1999) Plant J 20:401-412
Shippy et al., (1999) Mol. Biotech. 12:117-129
Slade and Knauf (2005) Transgenic Res 14: 109-115
Slade et al. (2005) Nature Biotech. 23:75-81
Smith et al., (2000) Nature 407:319-320
Stefanov et al., (1991) Acta Biologica Hungarica 42:323-330
Stickforth et al., (2003) Archiv Microbiol 179:409-415
Sun et al., (2005) Molecular Breeding 16, 209-218.
Tian et al., (2003) Plant Cell 15:1320-1332
Tian et al., (2004) Proc Natl Acad Sci USA 101:402-407
Tingay et al., (1997) Plant J. 11:1369-1376
Vishnevetsky et al., (1999) Trends Plant Sci 4:232-235
von Lintig et al., (1997) Plant J 12:625-634
Wan and Lemaux (1994) Plant Physiol. 104:37-48
Ward et al., (1996) In 46th Cereal Chemistry Conference, pp 145-149
Ward et al., (1997) 47th Aust Cereal Chem Conf:294-298
Waterhouse et al., (1998) Proc. Natl. Acad. Sci. 95:13959-13964
Welsch et al., (2000) Planta 211:846-854
Ye et al., (2000) Science 287:303-305
Zandomeneghi et al., (2000) J Agric Food Chem 48:2216-2221

1. A substantially purified and/or recombinant polypeptide selected from:
i) a polypeptide comprising an amino acid sequence as provided in SEQ ID NO:22,
ii) a polypeptide comprising an amino acid sequence as provided in SEQ ID NO:23,
iii) a polypeptide comprising an amino acid sequence as provided in SEQ ID NO:24,
iv) a polypeptide comprising an amino acid sequence which is at least 88% identical to any one of i) to iii), and
v) a biologically active fragment of any one of i) to iv), wherein the polypeptide has ε-cyclase activity.

2. An isolated and/or exogenous polynucleotide comprising a sequence of nucleotides selected from:
i) a sequence of nucleotides as provided in SEQ ID NO: 16,
ii) a sequence of nucleotides as provided in SEQ ID NO: 17,
iii) a sequence of nucleotides as provided in SEQ ID NO:18,
iv) a sequence of nucleotides as provided in SEQ ID NO: 19,
v) a sequence of nucleotides as provided in SEQ ID NO:20,
vi) a sequence of nucleotides as provided in SEQ ID NO:21,
vii) a sequence of nucleotides encoding a polypeptide of claim 1,
viii) a sequence of nucleotides which is at least 86% identical to any one of i) to vi), and
ix) a sequence which hybridises to any one of i) to vi) under stringent conditions.

3. A polynucleotide which, when present in a cell of a cereal plant, down-regulates the level of ε-cyclase activity in the cell when compared to a cell that lacks said polynucleotide.

4. The polynucleotide of claim 3, wherein the polynucleotide is selected from: an antisense polynucleotide, a sense polynucleotide, a catalytic polynucleotide, a microRNA and a double stranded RNA.

5. The polynucleotide of claim 4, which is a double stranded RNA (dsRNA) molecule, wherein the portion of the molecule that is double stranded is at least 19 basepairs in length.

6. A host cell comprising the polynucleotide of claim 3.

7. A transgenic plant, wherein the transgenic plant has increased or decreased expression of a polypeptide having ε-cyclase activity relative to a corresponding non-transgenic plant.

8. The transgenic plant of claim 7, the plant having been transformed with the polynucleotide according to claim 2.

9. A method of altering colour of flour produced from a cereal plant, the method comprising manipulating said plant such that the production of a polypeptide is modified when compared to a wild-type plant, wherein the polypeptide has ε-cyclase activity.

10. A method of genotyping a cereal plant, the method comprising detecting a nucleic acid of the plant or protein encoded thereby, wherein the nucleic acid molecule is genetically linked to, and/or comprises at least part of, a ε-cyclase gene.

11. The method of claim 10, wherein the method comprises determining the level of expression, and/or sequence, of a nucleic acid molecule of the plant encoding a polypeptide having ε-cyclase activity.

12. A method of selecting a cereal plant from a population of cereal plants, the method comprising;
i) genotyping said population of cereal plants using a method of claims 10, wherein said population of plants was obtained from a cross between two plants of which at least one plant comprises an allele of an ε-cyclase gene which confers upon said plant the ability to be used to produce flour, or a product obtained therefrom, with a desired colour, and
ii) selecting said cereal plant on the basis of the presence or absence of said allele.

13. A cereal plant, or progeny thereof, produced using the method of claim 10.

14. The plant of claim 13 which is a wheat or barley plant.

15. A method of producing seed, the method comprising;
a) growing the wheat and/or barley plant of claim 14, and
b) harvesting the seed.

16. A method of producing flour, wholemeal, or starch the method comprising;
a) obtaining seed from the plant of claim 13, and
b) extracting the flour, wholemeal, or starch.

17. Flour which comprises less than about 0.40 μg/g of lutein and/or lutein ester.

18. Flour which has a Minolta b* value of less than about 5.

19. Grain which comprises less than about 25 μg of lutein and/or lutein ester per 100 g of grain.


SEQUENCE LISTING (69 sequences)

SEQ ID NO: 1 - DNA, 723 bp, Triticum aestivum
aagggtatccgctatgmtctgaargcgcaagccgtttgggcgatctatgtatggtgcaggagaactgatgagctagtggatggccctaactcatcttacatcacgcccaaggcgctggatcggtgggagaagagattagaggatctcttcgaaggccgcccatatgatatgtacgacgcagccctctctgacacggcgtcaaagtttccaattgatatccagccgttcagagatatgattgaagggatgaggcttgacctttggaaatcgaggtatatgacctttgacgagctttacctctactgctactacgtcgctggcactgtcggtctcatgacggtaccggtggatggggattgctccggactcaaaggcctcagcagagagcgtgtacaatgccgcactggcccttggcattgccaaccagctcacaaacatcctccgagatgtaggagaagactcaaggagggggagaatataccttccactggacgaactggcacaggcgggtctgacagaagaggacatattcagagggaaagtgacggataaatggaggaggttcatgaagggccaaatccagcgcgccaggctcttctttgatgaggccgagaagggtgtcatgcatctagactctgcgagcagatggccggtcctggcatcgctgtggctgtacaggcagttcctggacgccatcgaggcgaacgactacaacaacttcac

SEQ ID NO: 2 - DNA, 719 bp, Triticum aestivum
caccrtcttatgactctggaggcgcaagctgtttgggcaatttatgtatggtgcaggagaactgatgagctagtggatggccctaactcgtcttacatcacgcccaaggcgctcgatcggtgggagaagagattggaggatctcttcgaaggccgcccgtatgatatgtatgacgcagccctctcagacacggcgtcaaagtttccaattgatatccagccattcagagacatgattgaagggatgaggctcgacctttggaaatcgaggtataggacctttgacgagctttacctctactgctactacgtcgctggcactgtcggtctcatgacggtaccggtgatggggattgctccggactcaaaggcctcagcagagagcgtgtacaatgccgcactggcccttggcattgccaaccagctcacgaacatcctccgagatgtaggagaagactcgaggagggggagaatataccttccactggacgagctggcacaggcgggcctgacagaggaggacatattcagagggaaagtgacggataaatggaggaggttcatgaagggccaaatccagcgcgccaggctcttcttcgatgaggccgagaagggcgtcatgcatctggactccgcgagcaggtggccggtcctggcttcgctgtggctgtacaggcagatccttgacgccatcgaggcgaacgactacaacaacttcac

SEQ ID NO: 3 - DNA, 720 bp, Triticum aestivum
cmtccgttatgactcctgtaaggcgcaagccgtttgggcaatctatgtatggtacaggagaactgatgagctagtggatggccctaactcatcttacatcacgcccaaggcgctcgatcggtgggagaagagattagaggatctcttcgaaggccgcccatatgatatgtatgatgcagccctctcagatacagcgtcaaagtttccaattgatatccagccattcagagacatgattgaagggatgaggctcgacctttggaaatcgaggtataggacctttgacgagctctacctctactgctactacgtcgctggcactgtcggtctcatgacggtaccggtgatggggattgctccggactcaaaggcctcagcagagagcgtgtacaatgccgcactggcccttggcattgccaaccagctcacaaacatcctccgagacgtaggagaagactcaagaagggggagaatataccttccactggacgagctggcacaggcgggtctgacagaagaggacatattcagagggaaagtgacggataaatggaggaggttcatgaaggggcaaatccagcgcgccaggctcttctttgacgaggccgagaagggcgtcatgcatctagactccgcgagcagatggccggtcctggcatcgctgtggctgtacaggcagatcctggacgccatcgaggcgaacgactacaacaacttcac

SEQ ID NO: 4 - DNA, 749 bp, Oryza sativa
ccttctacctaggtactcagcttatgactcctgaaaggcgcaaagctgtctgggcaatctatgtatggtgcagaagaactgatgaactggtagatggccctaactcgtcttacattacaccaaaggcacttgatcgatgggagaagagattagaagatctcttcgaaggcaggccatatgatatgtatgatgcagccctctcggacacagtgtcaaagtttccagtagatatccagccattcaaagacatgattgaaggaatgaggcttgacctgtggaaatcaaggtataggagctttgatgagctctacctctactgctactacgttgctggcacggttggtctcatgacagtaccggtgatggggattgcccccgactcgaaggcctcaaccgagagcgtgtacaacgctgcgctagctcttgggatcgccaaccagctgacgaatattctcagagacgtaggcgaagactcaaggaggggaagaatctaccttccattggatgaattggcagaggcaggtctgacagaagaagacatattcagagggaaagtgactgataaatggaggaagttcatgaagggacaaattctgcgtgccaggttattctttgatgaggcggagaagggcgttgcgcatctagactctgcgagtagatggccggttctggcatctttgtggttataccggcagatccttgatgctatcgaagcaaacgactacaacaacttcaccaagcgcgcgtat

SEQ ID NO: 5 - DNA, 221 bp, Triticum aestivum
acagcgtcaaagtttccaactgatatccaggtaagataagctgaggagataagttactgtgaaatggtgtatgaaacgccgctggatagaagttactgtacagtaaattagcaaggttcatttttgtccgttgtttctgtaacatcatgacctcttttctttacagccattcagagacatgattgaagggatgaggctcgacctttggaaatcgaggtata

SEQ ID NO: 6 - DNA, 221 bp, Triticum aestivum
acggcgtcaaagtttccaactgatatccaggtaagataagctgaggagataagttactgtgaaatggtgtatgaaacaccgccggatagaagttactgtatagtaacttagcaaggttcatttttgtccgttgtttctgtaacatcatgccctcttttctttacagccattcagagacatgattgaagggatgaggctcgacctttggaaatcgaggtata

SEQ ID NO: 7 - DNA, 185 bp, Triticum aestivum
acggcgtcaaagtttccaactgatatccaggtaagataagctgaggagataagttactgtgaaatggtgtatgaaactccgccggatagaagttattggatagtaacatcataacctcttttctttacagccgttcagagatatgattgaagggatgaggcttgacctttggaaatcgaggtata

SEQ ID NO: 8 - DNA, 989 bp, Triticum aestivum
gggaggatgaattcaaagatctcggcctggagagctgtattgagcatgtatggaaggatactgtcgtgtaccttgaccgtaacaagccgataatgattggccgagcatatggccgggtggaccgagacttgctgcacgaggagttgctgagaagatgcaatgaagctggtgttacatacctcaactcaaaggttgaacagataaaagaatctcctgatggacatcgagtagtatattgtggaaggggccacaagatactttgcaggcttgccattgttgcatctggagcagcatctggtaagcttctagagtatgaggttggaggaccacgtgtttgtgtgcagactgcatacggtgtagaagtcgaggttgaaagatatccgtacgatcccagcttaatggttttcatggactacagagattgtttcaaagagaagttcacacaccctgaggaagccaatccaacatttctctatgccatggccatgtcatctacacgagttttctttgaggaaacatgcttagcttcaaaagatgcaatgccttttgatctccttaagaagaggttgatgtctcggttggatgcgatgggagttcgtatcataaaagtatacgaggaggagtggtcttatattcctgttggaggatccttacctaacacagaccagaagaatcttgcatttggtgctgcagcgagtatggtccatcctgcaactggatattcggtggtcagatctttgtctgaagctcctagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacgaaggcattcaaacattcttcgagagctttttccggttgcccaaatggatgtgg

SEQ ID NO: 9 - DNA, 1001 bp, Triticum aestivum
gggaattcgattgggaggatgaattcaaagatctcggcctggagagctgtattgagcatgtatggaaggatactgtcgtgtaccttgaccgtaacaagccgataatgattggccgagcatatggccgggtggaccgagacttgctgcacgaggagttgctgagaagatgcaatgaagctggtgttacatacctcaactcaaaggttgaacagataaaagaatctccagatggacatcgagtggtatattgtggaaggggccgcaagatactttgcaggcttgccattgttgcatctggagcagcatctggtaaacttctagagtatgaggttggaggaccacgtgtttgtgtgcagactgcatacggtgtagaagtcgaggttgaaagatatccgtacgatcccagcttaatggttttcatggactacagagattgtttcaaagagaagttcacacaccctgaggaagccaatccaacatttctctatgccatggccatgtcatctacacgagttttctttgaggaaacatgcttagcttcaaaagatgcaatgccttttgatctccttaagaagaggttgatgtctcggttggatgcgatgggagttcgtatcataaaagtatacgaggaggagtggtcttgtattcctgttggaggatccttacctaacacagaccagaagaatcttgcatttggtgctgcagcgagtatggtccatcctgcaactggatattcggtggtcagatctttgtctgaagctcctagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacgaaggcattcaaacattcttcgagagctttttccggttgcccaaatggatgtgg

SEQ ID NO: 10 - DNA, 988 bp, Triticum aestivum
gggaggatgaattcaaagatctcggcctggagagctgtattgagcatgtatggaaggatactgtcgtgtacctagaccgtaacaagccgataatgattggccgagcatatggccgggtggaccgagacttgctgcacgaggagttgctgagaagatgcaacgatgctggtgttacatacctcaactcaaaggttgaacagataatagaatctccagatggacatcgagtagtatattgtgaaagggaccgcaagatactttgcaggcttgccattgttgcatctggagcagcatctggtaagcttctagagtatgaggttggaggaccacgtgtttgtgtgcagactgcatatggtgtagaagtcgaggttgaaagatatccgtacaaccccagcttaatggttttcatggactacagagattgtttcaaagagaagttctcacaccctgaggaagccaatccaacatttctctatgccatggccatgtcatccacacgagttttctttgaggaaacatgcttagcttcaaaagatgcaatgccttttgatctccttaagaagaggttgatgtctcggttggatgcgatgggagttcgtatcgtaaaagtatacgaggaggagtggtcttatattcctgttggaggatccttacctaacacagaccagaagaatcttgcatttggtgctgcagcgagtatggtccatcctgcaactggatattcagtggtcagatctttgtctgaagctccaagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacaaaggcatccaaacattctttgaaacctttttcggttgcccaaatggatgtgg

SEQ ID NO: 11 - DNA, 1050 bp, Oryza sativa
ccattcacgaacaactacggtgtgtgggaggatgaattcaaagacctgggcctggagagctgcattgaacatgtctggaaggatactatcgtgtacctagacggtaacaagccaataatgattggccgtgcgtatggcagggttcacagggacttgctgcacgaggagttgctgagacgatgctatgacgctggcgtcacatacctcagctcgaaggtggacaagatcatggaatctcctgatggacatcgggtagtctgttgtgaaggggatcgtgaggtactttgcaggcttgccattgttgcatctggggcagcatctggtaggcttctagagtacgaggttggtggtccgcgtgtttgtgtgcagactgcatatggtgtcgaagtcgaggtggaaaacaatccatatgatcccagcttaatggttttcatggactacagagattgcttcaaagacaaattctcacatcctgagcaaggaaatccaacgttcctctatgccatgcccatgtcatccacacgaattttctttgaggaaacatgcctagcttctaaagaagcaatgccctttgacctccttaaaaagcggttgatgtctcggttggatgcaatgggagttcatattcgaaaagtatacgaggaggaatggtcctacattcctgttggagggtccttaccaaatacagaccagaaaaatctcgcatttggtgcggcagcaagtatggtgcatcctgcaaccggatactcggtggttagatcattgtctgaagctccaagatatgcatctgtgatatctgatatcttgagaaaccgtgtctaccctggagaatatttgcctggaacctctcaaagttccagtccatcaatgcttgcatggagaacattatggccccaagaacggaaacgtcaacgatcattcttcctttttgggctggctttgataatccaactgaataacgaaggcattcagacattctttgaaacctttttccggttgcccaaatggatgtggcgaggattccttggttcgacgctttcttcagtggat

SEQ ID NO: 12 - DNA, 350 bp, Triticum aestivum
ggggggggtwtgagggttggaggaccacgtgtttgtgtgcagactgcatacggtgtagaagtcgaggtacacaccaaccctgcacaaagtgccccttctgattcgtggtttatttcatgatttgcatgtcacttcattcatgtcattcgagctgagaaattcataggttgtttctgcaccagttcaatttttcagaatgatgtcaaaatttcaggttgaaagatatccgtacgatcccagcttaatggttttcatggactacagagattgtttcaaagagaagttcacacaccctgaggaagccaatccaacatttctctatgccatggccatgtcatcaatcactagtg

SEQ ID NO: 13 - DNA, 350 bp, Triticum aestivum
ccccyctgagttgaggttggaggaccacgtgtttgtgtgcagactgcatacggtgtagaagtcgaggtacacaccaaccctgcacaaagtgctccttctgattcgtggtttatttcatgatttgcatgtcacttcattcatgtcattcgagctgagaaattcatatgttgtttgtgcaccagttcaatttttcagaatgatgtcaaaatttcaggttgaaagatatccgtacgatcccagcttaatggttttcatggactacagagattgtttcaaagagaagttcacacaccctgaggaagccaatccaacatttctctatgccatggccatgtcatcaatcactagtg

SEQ ID NO: 14 - DNA, 350 bp, Triticum aestivum
gggctctgagttgaggttggaggaccacgtgtttgtgtgcagactgcatatggtgtagaagtcgaggtacacagcaaccctgcacaaagtgctccttctgattcgtggtttatttcatgatttgcatatgtcacttcattcatgtcactcgagctgagaaattcatacgtggtttgtgccccagttcaatttttcagaatgatgtcaaaatttcaggttgaaagatatccgtacaaccccagcttaatggttttcatggactacagagattgtttcaaagagaagttctcacaccctgaggaagccaatccaacatttctctatgccatggccatgtcatcaatcactag

SEQ ID NO: 15 - DNA, 400 bp, Oryza sativa
gttgcatctggggcagcatctggtaggcttctagagtacgaggttggtggtccgcgtgtttgtgtgcagactgcatatggtgtcgaagtcgaggtatgcacaactcctatagaacttactcccctcgagagtttagaatagtttcccttctgtcgtcgtgtttctacgagcattttattcatattgtttcaagctgaaaattttagatatgttatttgggcacatgtcattcatctttcacaatgatttgaaaattttcaggtggaaaacaatccatatgatcccagcttaatggttttcatggactacagagattgcttcaaagacaaattctcacatcctgagcaaggaaatccaacgttcctctatgccatgcccatgtcatccacacgaattttct

SEQ ID NO: 16 - DNA, 4642 bp, Triticum aestivum
tatcccggataccctctccaaaacctctccctccacaacgcctcctcctccgctcctccctaggcagctagcctctccgcctccctcccttccggccagccagccagctcgcaattcccgcaactcgcgccgccgcgagggcaatggagtccaccggcgccgccatctcggcgccgttcggctgccgcgcgttgcgctgggccgggcagcggcctctccgggcggccgatgccaggagaaggcgtgtgggttcgggccccgggccggagcagtggaggagcttgaaggcgagctgcgtggccacggagaagcccgacgagaaggcggcgccggggctggagttcgccgacgacgaggactacgtcaagggcggcggcggcgagctgctctacgtgcaaatgcaggccaccaaggccatggaaagccagtccaagatcgcctccaaggttctctctctatccttttctgtactgctacggctactgctactagtatcttgttctttgttttattcccttatttaattgctgatttctatgcatgctttgttgatcttgaatatactactccactacttaagtgattcttacccacaattagcgttgtgtgaacttccagcgttatggcgaataaaattaaaatccctaacattagtctgtttctttcttgcgatgtacagtagtaacattagactgtctgaaagatgaaggattcccataagcaccacagtagcaaaaattaatctgggtactattgtatactatactactccctccgtatcaaaatataagactgccttttgacactattatagtgtccaaaggcagaaaatacataggagctcctgggtgatccacaccctatatgaatattaaacgtaaaaaataccaaaaaaatcaaaaaaatctgaaatttggggatattaaacctgggttcccaatctactcctgtgtgaaattttatgaaaaaataccagcacacatatccgtggcgaaggaaatactatccgaacaaaaatccatccaaacagttttttctatacgtagaaattttttgtctttttcgccacgaatagttttcctggtatttttttcacgaaattttacgcgggagtagatcgcgaacccaagtttgatatcccaaaatcttagattttttttcaaatttctcggtatttttttgaatttaatgttcgtgtaggggtgtgaagcaaccgggtgctacaaatccgcttccatccaaaggcgtcttatatttggatatagagggagtagaacgcaagtggcattcaaatgcacgtcatgctagtactaccacggcgttgctcatttttgacaaatcaaagcaggaatagtcttgggtcgcccatgcttgtcattcctgccttcctggcaggagaagagtacacaatgtatttggtcttgcgttaattaattgctccttagggaaatgtctttggttgtgcgttcaytaattacaccttaggaaaatgataacatgtatacaatccgtgatgttgaatttgttgattcggatgtccattctattatcccagtgtcatgcctttggcttgctttctttggaaacgccctttcactaagtatatcattcaataattcctacgtagcaaggcgccggatcttagtgggttacgaacgtcctaattttaccttcatgttggtttgactttgttgcatgtgcttaatagttattactaggaaacaagctcccagtctggttctaacgaattttaattgaactgtttgcacagacagcaaataactagaccgggcattaagaatgtttcaatccttcttttattgatactctgtatagttctttttcttcaaggttaataattaagtttcgaagttcatcgtcattgtgttaacaattttttgtctggtagcatccttcaagtagttgattatgtcttctgaatctttctttgcagctgttgcctatagctgatgaaacttcagtgcttgatttggttatcattggctgtggtccagccggcctatctctggctgcagartcagcaaataaaggactcactgttggtctcattggccctgatcttccattcrcaarcaattacggtgtatgggaggacgaattcaaaggtagccattattcaccactgtgaaatgtagcagcatgcttggataacacctttaccagcattatcttaaaaagtaatacgtttttatctttttatttcccagatctcggcctggagagctgtattgagcatgtatggaaggatactgtcgtgtacctagaccgtaacaagccgataatgattggccgagcatatggccgggtggaccgagacttgctgcacgaggagttgctgagaaggtagacattctattgtaacgatcttggaattcagttgcatcaggcarcatgatatatatgactgttcctagtaacaataattaaataattggcagatgcaacgatgctggtgttacatacctcaactcaaaggttgaacagataatagaatctccagatggacatcgagtagtatattgtgaaagggaccgcaagatactttgcaggcttgccattgttgcatctggagcagcatctggtaagcttctagagtatgaggttggaggaccacgtgtttgtgtgcagactgcatatggtgtagaagtcgaggtacacagcaaccctgcacaaagtgctccttctgattcgtggtttatttcatgatttgcatatgtcacttcattcatgtcactcgagctgagaaattcatacgtggtttgtgccccagttcaatttttcagaatgatgtcaaaatttcaggttgaaagatatccgtacaaccccagcttaatggttttcatggactacagagattgtttcaaagagaagttctcacaccctgaggaagccaatccaacatttctctatgccatggccatgtcatccacacgagttttctttgaggtctacatgaatttttttagcagtgtgcttctgcaatggacacgtttccataacgatgggtcactgaccaagataatttatcacaggaaacatgcttagcttcaaaagatgcaatgccttttgatctccttaagaagaggttgatgtctcggttggatgcgatgggagttcgtatcgtaaaagtatacgaggaggtaacgagtttggagttgatatccatactgatttatcttgcacaggtgtgctgaatttctgttgagtcttgatttcaggagtggtcttatattcctgttggaggatccttacctaacacagaccagaagaatcttgcatttggtgctgcagcgagtatggtccatcctgcaactggtacatacaaatcctcaacttgaactcgtcattctttattatttgacatgcatattgacaatattttagaaaattcataggatattcagtggtcagatctttgtctgaagctccaagatatgcttctgtgatatctgatatcttacgaaatcgtgtctattctggacaatatttgcctggaagttctgaaatgtccagtccatcaatgcttggtacagatttcttcccttgttcactaaattcaacagatagaataaaatcacttaaagatgttttgcagaataggagaaatgagcacaagtgtctgctgctaaccactgtctgatatttcagcatggggaacactatggcctcaagaacggaaacgtcagcgctcattcttcctctttggattggccttgataattcaactggataacaaaggcatccaaacattctttgaaacctttttccggttacccaaatggtaattacacccgctcgatgcatttattgcttgttttcaggtcccggactaagtatttccttttttcgcgagtacgcaaggatgtgtatcttcccattgatagaaaaaagtttgaacaagtaaatatttccttgcgcgtgacttttgatggatcgatctcttgaacaggatgtggcaaggattccttggttcgacgctttcgtcggtggatctcatgctgtttgcactctacatgtttgcaattgcgccaaacactctgcgaatgaacctcgtcagacacctcctctcggacccgactggttcggcaatgatcaggacctacctgaccttgtaaaaccattcatttgactagcctgcgagaaatctagaaagtgtacagttttgtagttgtacataccatagttagagatgttggaggatgtcacctgccgtgccatgtgttgcctctgcctatgcctagtagtggagtaaagcgtggatctttgtatagttgaagtggccatggtttaattttaggtttggtctgggtcaagcttctccgggagccaaaagagggagcgtgcaagaatagccaagagtggtggtctaatctggtaccccctccctccctaggtcatttaacggcggcatgcacggggaggatctttgtaggcaaggaaggaagggagcaggaggaagaggagaagggacgcttccgtcgttccctcaacctctctgcctctcctttctctttctcttgttgcaacatttgacatccatccaagtgatcggagcatcgccggcatccattcccggtggaaccgatgatgcagggaagccccctgagtcccgatgagcacc

SEQ ID NO: 17 - DNA, 4391 bp, Triticum aestivum
tatcccggataccctctccaaaacctctccctccacaacgcctcctcctccgctcctccctaggcagctagcctctccgcctccctccccttccggccagccagccagctcgcaattccctgaacctgcgccgccgcgggggcaatggagtccaccggcgccgccatctcggcgccgttcggctgccgcgcgttgcgctgggccgggcagcggcctctccggccggccgatggcaggagaaggcgggtgggtccgggccccgggccggagaagtggaggagcttgaaggcgagctgcgtggccacggagaagcccgacgagaaggcggcgccggggctaggggtggagtttccgacgaggaggactacgtcaagggcggcggcggtgagctgctctacgtgcaaatgcaggccaccaaggccatggaaagccagtccaagatcgcctccaaggttctctctctatccttttatgtactgctaccgctagtctgctactgctagtggtatcttgttctttgttttattcccttatttaattgctgatttctatgcatgttttgttgatcttgaatatactactctagtacttaagtgattctttcccccaattagcgttgtgtgaacttccagcgttatggcgaataaaattaaaatccctaacattagtctgtttctttcttgcgatgtacagtagtaacattagactgtttgaaagatgaaggattcccataaacaccacagtagcaaaaattaatctgggtactattgcatactatactactccctccgtatcaaaatataagaccagaaaatacataggagctcttgggtgctccacaccctatatgaatattaaacgtaaaaaatacccaaaaaattaaaaaatctgaaatttggggatattaaacctgggttcttaatctactcccgtgtgaaattttgtgaaaaaataccagcacacgtatccgtggcgaaggaaatattgtccgaacaaaaatccatccaaacagttttttctatacataggaattttttgtcttttttgccacgaatatgtttcctggtatttttttcacgaaattttacacgggagtagatcgggaacccaggtttgatatcccaaaatcttagttttttttcaaatttctcggtatttttttaaatttaatgttcgtgtaggggtgtggagcaaccgggtgctacaaatccgcttccatccaaaggggtcttatatttggatatagagggagtagaacgcaagtggcattcaaatgcacgtcatgctagtactaccatggcgttgctcatttttgacaaatcagggatagtcttgggtcgcccatgcttgtcattcctgccttcctggcaggagcagagtacacaatgtatttggtcttgcgttaattaattgctccttagggacatgtctttggtcgtgcctttgacttgctttctttggaaacgccctttcactaagtatatcattcaataattcctacatagcaagacgcggatcttagtgggttacgaacgtcattgtgtacatgtttcaatccttcttttactgatactctgtacagttctttttcttcaaggttaattaattaagtttcgaagttcatcgtcattttgttaacaattttttgtctggtagcatccttcaagtagttgattatgtcttctgaatctttctttgcagctgttgcctatagctgatgaaacttcagtgcttgatttggttatcattggctgtggtccagccggcctatctctggctgcagaatcagcaaagaaaggactcactgttggtctcattggccctgatcttccattcacaaacaattacggtgtatgggaggacgaattcaaaggtagccattagtcgcagctgtgaaatgcagcaccatgcttggataacatctttaccagcattatcataaagagtgatacgtttttatctttttatttcccrgatctcggcctggagagctgtattgagcatgtatggaaggatactgtcgtgtaccttgaccgtaacaagccgataatgattggccgagcatatggccgggtggaccgagacttgctgcacgaggagttgctgagaaggtacattctatcataacgatcttggaattcagtcgcgtcaggcagcatcatatatatgattgttcctagtaatgataattaaacaattggcagatgcaatgaagctggtgttacatacctcaactcaaaggttgaacagataaaagaatctcctgatggacatcgagtagtatattgtggaaggggccacaagatactttgcaggcttgccattgttgcatctggagcagcatctggtaagcttctagagtatgaggttggaggaccacgtgtttgtgtgcagactgcatacggtgtagaagtcgaggtacacaccaaccctgcacaaagtgctccttctgattcgtggtttatttcatgatttgcatgtcacttcattcatgtcattcgagctgagaaattcatatgttgtttgtgcaccagttcaatttttcagaatgatgtcaaaatttcaggttgaaagatatccgtacgatcccagcttaatggttytcatggactacagagattgtttcaaagagaagttcacacaccctgaggaagccaatccaacatttctctatgccatggccatgtcatctacacgagttttctttgaggtctacatgaatttttttagcagtgtgtttctgcaatggacacgtttccataaggataggtcactgaccaagataatttatcataggaaacatgcttagcttcaaaagatgcaatgccttttgatctccttaagaagaggttgatgtctcggttggatgcgatgggagttcgtatcataaaagtatacgaggaggtaacgagtttggagttgatatccatactggtttatcttgcacaggtgtgctgaatttctgttgagtcttgatttcaggagtggtcttatattcctgttggaggatccttacctaacacagaccagaagaatcttgcatttggtgctgcagcgagtatggtccatcctgcaactggtacatacaaatcctcaactttaactcgtcattctttattatttgacatgcatattgacaatattgtagaaaattcataggatattcggtggtcagatctttgtctgaagctcctagatatgcttctgtgatatctgatatcttacgaaatcgtgtctattctggacaatatttgcctggaagttctgaaatgtccagtccatcaatgcttggtacagatttcttcccttgttcactaaattcaacagatagaataaaatcacttaaagatgttttgcagaataggagaaatgagcagaagtatctgctgctaaccactgtctgatatttcagcatggggaacactatggcctcaagaacggaaacgtcagcgctcattcttcctctttggattggccttgataattcaactggataacgaaggcattcaaacattcttcgagagctttttccggttacccaaatggtaattatgcccgctcgatgcatttactgcttgttttcaggccctggaccaagtatttcctttttcgcgaatacaatacgcaaggatgcgtatctttccattgacagaaaaagtttgaacaagtaaatatttccttgcgcgtgacttttgatggatcgatctcttgaacaggatgtggcgaggattccttggttcgacgctttcgtcagcggatctcatgctgtttgcactctacatgtttgcaattgcgccaaacactttgcgaatgaacctcgtcagacacctcctctcggacccgactggttcggcaatgatcaggacctacctgaccttgtaaaaccatttgatgactagtctgcgagaaatctagaaagtgtacagttttgtagttgtacataacatagttagagatgttgggggatatcacctgccatgtgttgcctctgcctatgcctagtagtggagtaaagcgtggatcttgtatagttgaagttgccatggtttaattttaggtttggtctgggtcaagcttctccgggagccaaaagtgggagcgtgcaagaatagccaagagtgctggtctaatctggtaccccctccccccctaggtcatttaacggcggcatgcacggggaggatctttgtaggcaaggaagggagcaggaggaagaggaggagaagggacgcttccgtcgttccctcaacctctctgcctctcctttctctttctcttggtggccgccccttgtaacatttggcatccatccaactaatcggagcatcgctgggatccattcgcagtggaaccgatgatgcaggggagccccctgagtcccgatgagcacc

SEQ ID NO: 18 - DNA, 4435 bp, Triticum aestivum
tatcccggataccctctccaaaacctctccctccacaacgcactccctcctcctcctccgctcctccctaggcagctagcctctccgcctccctccccttccggccagccagccacctcgcaattccctgaacctgcgccrccgcgggggcaatggagtccaccggcgcygccatctcggcgccgttcggctgccgcgcgttgcgctgggccgggcagcggcctctccggccggccgatgccaggagaaggcgggtgggtccgggccccgggccggagcagtggaggagcttgaaggcgagctgtgtggccacggagaagcccgacgagaaggcggcgccggggctgggggtagacttcgccgacgaggaggactacgtcaagggcggcggcggtgagctgctctacgtgcaaatgcaggccaccaaggccatggaaagccagtccaagatcgcctccaaggttctctctctatccttttatgtactgctaccgctaatttgctactgctagtggtatcttgttctttgttttattcccttatttaattgctgatttctatgcatgttttgttgatcttgaatatactactctagtactcaagtgattctttcccccaattagcgttgtgtgaacttccagcgttatggcgaataaaattaaaatccctaacattagtctgtttctttcttgcgatgtacagtagtaacattagactgtttgaaagatgaaggattcccataagcaccacagtagcaaaaattaatctgggtactattgcatactatactactccctccgtatcaaaatataagactgccttttgacactattatagtgtccaaaggcagaaaatacataggagctcctgggtgatccacaccctatatgaatattaaacgtaaaaaaataccaaaaaaatcaaaaaaatctgaaatttggggatattaaacctgggttcccaatctactcctgtgtgaaattttatgaaaaaataccagcacacatatccgtggcgaaggaaatactatccgaacaaaaatccatccaaacagttttttctatacatagaaattttttgtctttttcgccacgaatagttttcctggtatttttttcacgaaattttacgcgggagtagatcgcgaacccaagtttgatatcccaaaatcttagattttttttcaaatttctcggtatttttttgaatttaatgttcgtgtaggggtgtgaagcaaccgggtgctacaaatccgcttccatccaaagggggcttatatttggatatagagggagtagaacgcaagtggcattcaaatgcacgtcatgctagtactaccatggcgttgctcatttttgacaaatcaaagcaggaatagtcttgggtcgcccatgcttgtcattcctgccttcctggcaggagaagagtacacaatgtatttggtcttgcgttaattaattgctccttagggacatgtctttggtcgtgcctttgacttgctttctttggcaacgccctttcactaagtatatcattcaataattcctacatagcaagacgcggatcttagtgggttacaaacgtcattgtgtacatgtttcaatccttcttttactgatactctgtacagttctttttcttcaaggttaattaattaagtttcgaagttcatcgtcattttgttaacaattttttgtctggtagcatccttcaagtagttgattatgtcttctgaatctttctttgcagctgttgcctatagctgatgaaacttcagtgcttgatttggttatcattggctgtggtccagccggcctatctctggctgcagaatcagcaaagaaaggactcactgttggtctcattggccctgatcttccattcacaaacaattacggtgtatgggaggacgaattcaaaggtagccattagtcgcagttgtgaaatgcagcaccatgcttggataacatctttaccagcattatcttaaaaagtgatacgtttttatctttttatttcccagatctcggcctggagagctgtattgagcatgtatggaaggatactgtcgtgtaccttgaccgtaacaagccgataatgattggccgagcatatggccgggtggaccgagacttgctgcacgaggagttgctgagaaggtacattcgatcttggaattcagtcgcgtcaggcagcatcatatatatgattgtttctagtaatgataattaaacaattggcagatgcaatgaagctggtgttacatacctcaactcaaaggttgaacagataaaagaatctccagatggacatcgagtggtatattgtggaaggggccgcaagatactttgcaggcttgccattgttgcatctggagcagcatctggtaaacttctagagtatgaggttggaggaccacgtgtttgtgtgcagactgcatacggtgtagaagtcgaggtacacaccaaccctgcacaaagtgccccttctgattcgtggtttatttcatgatttgcatgtcacttcattcatgtcattcgagctgagaaattcataggttgtttctgcaccagttcaatttttcagaatgatgtcaaaatttcaggttgaaagatatccgtacgatcccagcttaatggttttcatggactacagagattgtttcaaagagaagttcacacaccctgaggaagccaatccaacatttctctatgccatggccatgtcatctacacgagttttctttgaggtctacatgaatatttttttagcagcgtgtttctgcaatggacacgtttccataaggataggtcactgaccaagataatttatcacaggaaacatgcttagcttcaaaagatgcaatgccttttgatctccttaagaagaggttgatgtctcggttggatgcgatgggagttcgtatcataaaagtatacgaggaggtaacgagtttggagttgatatccatactggtttatcttgcacaggtgtgctgaatttctgttgagtcttgatctcaggagtggtcttatattcctgttggaggatccttacctaacacagaccagaagaatcttgcatttggtgctgcagcgagtatggtccatcctgcaactggtacatacaaatcctcaacttgaactcgtcattctttattatttgacatacatattgacaatattgtagaaaaaaattcataggatattcggtggtcagatctttgtctgaagctcctagatatgcttctgtgatatctgatatcttacgaaatcgtgtctattctggacaatatttgcctggaagttctgaaatgtccagtccatcaatgcttggtacagatttcttcccttgttcactaaatccaacagatagaataaaatcacttaaagatgttttgcagaataggagaaatgagcagaagtatctgctgctaaccattgtctgatatttcagcatggggaacactatggcctcaagaacggaaacgtcagcgctcattcttcctctttggattggccttgataattcaactggataacgaaggcattcaaacattcttcgagagctttttccggttacccaaatggtaattatgcccgctcgatgcatttactgcttgttttcaggccctggactaagtatttcctttttcaagaatacaatacgcaaggatgcgtatctttccattgatagaaaaagtttgaacaagtaaatatttccttgcgcgtgatttttgatggatcgatctcctgaacaggatgtggcgaggattccttggttcgacgctttcgtcagcggatctcatgctgtttgcactctacatgtttgcaattgcgccaaacactttgcgaatgaacctcgtcagacacctcctctcggacccgactggttcggcaatgatcaggacctacctgaccttgtaaaaccatttgatgactagtctgcgagaaatctagaaagtgtacagttttgtagttgtacataacatagttagagatgttgggggatatcacctgccatgtgttgcctctgcctatgcctagtagtggagtaaagcgtggatcttgtatagttgaagtggccatggtttaattttaggtttggtctgggtcaagcttctccgggtgccaaaagtgggagcgtgcaagaatagccaaatgtgctggtctaatctggtaccccctcgccccctaggtcatttaacggcggcatgcacggggaggatctttgtaggcaaggaagggagcaggaggaagaggaggagaagggacgcttccgtcgttccctcaacctctctgcctctcctttctctttctcttggtggccgccccttgtaacatttggcatccatccaactgatcggagcatcgccggcatccattcccggtggaaccgatgatgcagggaagccccctgagtcccgatgagcacc

SEQ ID NO: 19 - DNA, 1606 bp, Triticum aestivum
atggagtccaccggcgccgccatctcggcgccgttcggctgccgcgcgttgcgctgggccgggcagcggcctctccgggcggccgatgccaggagaaggcgtgtgggttcgggccccgggccggagcagtggaggagcttgaaggcgagctgcgtggccacggagaagcccgacgagaaggcggcgccggggctggagttcgccgacgacgaggactacgtcaagggcggcggcggcgagctgctctacgtgcaaatgcaggccaccaaggccatggaaagccagtccaagatcgcctccaagctgttgcctatagctgatgaaacttcagtgcttgatttggttatcattggctgtggtccagccggcctatctctggctgcagaatcagcaaataaaggactcactgttggtctcattggccctgatcttccattcgcaagcaattacggtgtatgggaggacgaattcaaagatctcggcctggagagctgtattgagcatgtatggaaggatactgtcgtgtacctagaccgtaacaagccgataatgattggccgagcatatggccgggtggaccgagacttgctgcacgaggagttgctgagaagatgcaacgatgctggtgttacatacctcaactcaaaggttgaacagataatagaatctccagatggacatcgagtagtatattgtgaaagggaccgcaagatactttgcaggcttgccattgttgcatctggagcagcatctggtaagcttctagagtatgaggttggaggaccacgtgtttgtgtgcagactgcatatggtgtagaagtcgaggttgaaagatatccgtacaaccccagcttaatggttttcatggactacagagattgtttcaaagagaagttctcacaccctgaggaagccaatccaacatttctctatgccatggccatgtcatccacacgagttttctttgaggaaacatgcttagcttcaaaagatgcaatgccttttgatctccttaagaagaggttgatgtctcggttggatgcgatgggagttcgtatcgtaaaagtatacgaggaggagtggtcttatattcctgttggaggatccttacctaacacagaccagaagaatcttgcatttggtgctgcagcgagtatggtccatcctgcaactggatattcagtggtcagatctttgtctgaagctccaagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacaaaggcatccaaacattctttgaaacctttttccggttgcccaaatggatgtggcaaggattccttggttcgacgctttcgtcggtggatctcatgctgtttgcactctacatgtttgcaattgcgccaaacactctgcgaatgaacctcgtcagacacctcctctcggacccgactggttcggcaatgatcaggacctacctgaccttgtaaa

SEQ ID NO: 20 - DNA, 1611 bp, Triticum aestivum
atggagtccaccggcgccgccatctcggcgccgttcggctgccgcgcgttgcgctgggccgggcagcggcctctccggccggccgatggcaggagaaggcgggtgggtccgggccccgggccggagaagtggaggagcttgaaggcgagctgcgtggccacggagaagcccgacgagaaggcggcgccggggctaggggtggagtttgccgacgaggaggactacgtcaagggcggcggcggtgagctgctctacgtgcaaatgcaggccaccaaggccatggaaagccagtccaagatcgcctccaagctgttgcctatagctgatgaaacttcagtgcttgatttggttatcattggctgtggtccagccggcctatctctggctgcagaatcagcaaagaaaggactcactgttggtctcattggccctgatcttccattcacaaacaattacggtgtatgggaggacgaattcaaagatctcggcctggagagctgtattgagcatgtatggaaggatactgtcgtgtaccttgaccgtaacaagccgataatgattggccgagcatatggccgggtggaccgagacttgctgcacgaggagttgctgagaagatgcaatgaagctggtgttacatacctcaactcaaaggttgaacagataaaagaatctcctgatggacatcgagtagtatattgtggaaggggccacaagatactttgcaggcttgccattgttgcatctggagcagcatctggtaagcttctagagtatgaggttggaggaccacgtgtttgtgtgcagactgcatacggtgtagaagtcgaggttgaaagatatccgtacgatcccagcttaatggttttcatggactacagagattgttccaaagagaagttcacacaccctgaggaagccaatccaacatttctctatgccatggccatgtcatctacacgagttttctttgaggaaacatgcttagcttcaaaagatgcaatgccttttgatctccttaagaagaggttgatgtctcggttggatgcgatgggagttcgtatcataaaagtatacgaggaggagtggtcttatattcctgttggaggatccttacctaacacagaccagaagaatcttgcatttggtgctgcagcgagtatggtccatcctgcaactggatattcggtggtcagatctttgtctgaagctcctagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacgaaggcattcaaacattcttcgagagctttttccggttgcccaaatggatgtggcgaggattccttggttcgacgctttcgtcagcggatctcatgctgtttgcactctacatgtttgcaattgcgccaaacactttgcgaatgaacctcgtcagacacctcctctcggacccgactggttcggcaatgatcaggacctacctgaccttgtaa

SEQ ID NO: 21 - DNA, 1611 bp, Triticum aestivum
atggagtccaccggcgctgccatctcggcgccgttcggctgccgcgcgttgcgctgggccgggcagcggcctctccggccggccgatgccaggagaaggcgggtgggtccgggccccgggccggagcagtggaggagcttgaaggcgagctgtgtggccacggagaagcccgacgagaaggcggcgccggggctgggggtagacttcgccgacgaggaggactacgtcaagggcggcggcggtgagctgctctacgtgcaaatgcaggccaccaaggccatggaaagccagtccaagatcgcctccaagctgttgcctatagctgatgaaacttcagtgcttgatttggttatcattggctgtggtccagccggcctatctctggctgcagaatcagcaaagaaaggactcactgttggtctcattggccctgatcttccattcacaaacaattacggtgtatgggaggacgaattcaaagatctcggcctggagagctgtattgagcatgtatggaaggatactgtcgtgtaccttgaccgtaacaagccgataatgattggccgagcatatggccgggtggaccgagacttgctgcacgaggagttgctgagaagatgcaatgaagctggtgttacatacctcaactcaaaggttgaacagataaaagaatctccagatggacatcgagtggtatattgtggaaggggccgcaagatactttgcaggcttgccattgttgcatctggagcagcatctggtaaacttctagagtatgaggttggaggaccacgtgtttgtgtgcagactgcatacggtgtagaagtcgaggttgaaagatatccgtacgatcccagcttaatggttttcatggactacagggattgtttcaaagagaagttcacacaccctgaggaagccaatccaacatttctctatgccatggccatgtcatctacacgagttttctttgaggaaacatgcttagcttcaaaagatgcaatgccttttgatctccttaagaagaggttgatgtctcggttggatgcgatgggagttcgtatcataaaagtatacgaggaggagtggtcttgtattcctgttggaggatccttacctaacacagaccagaagaatcttgcatttggtgctgcagcgagtatggtccatcctgcaactggatattcggtggtcagatctttgtctgaagctcctagatatgcttctgtgatatctgatatcttacgaaatcgtgtctattctggacaatatttgcctggaagttctgaaatgtccagtccatcaatgcttgcatggggaacactatggcctcaagaacggaaacgtcagcgctcattcttcctctttggattggccttgataattcaactggataacgaaggcattcaaacattcttcgagagctttttccggttgcccaaatggatgtggcgaggattccttggttcgacgctttcgtcagcggatctcatgctgtttgcactctacatgtttgcaattgcgccaaacactttgcgaatgaacctcgtcagacacctcctctcggacccgactggttcggcaatgatcaggacctacctgaccttgtaa

SEQ ID NO: 22 - protein, 534 aa, Triticum aestivum
MESTGAAISAPFGCRALRWAGQRPLRAADARRRRVGSGPGPEQWRSLKASCVATEKPDEKAAPGLEFADDEDYVKGGGGELLYVQMQATKAMESQSKIASKLLPIADETSVLDLVIIGCGPAGLSLAAESANKGLTVGLIGPDLPFASNYGVWEDEFKDLGLESCIEHVWKDTVVYLDRNKPIMIGRAYGRVDRDLLHEELLRRCNDAGVTYLNSKVEQIIESPDGHRVVYCERDRKILCRLAIVASGAASGKLLEYEVGGPRVCVQTAYGVEVEVERYPYNPSLMVFMDYRDCFKEKFSHPEEANPTFLYAMAMSSTRVFFEETCLASKDAMPFDLLKKRLMSRLDAMGVRIVKVYEEEWSYIPVGGSLPNTDQKNLAFGAAASMVHPATGYSVVRSLSEAPRYASVISDILRNRVYSGQYLPGSSEMSSPSMLAWGTLWPQERKRQRSFFLFGLALIIQLDNKGIQTFFETFFRLPKWMWQGFLGSTLSSVDLMLFALYMFAIAPNTLRMNLVRHLLSDPTGSAMIRTYLTL

SEQ ID NO: 23 - protein, 536 aa, Triticum aestivum
MESTGAAISAPFGCRALRWAGQRPLRPADGRRRRVGPGPGPEKWRSLKASCVATEKPDEKAAPGLGVEFADEEDYVKGGGGELLYVQMQATKAMESQSKIASKLLPIADETSVLDLVIIGCGPAGLSLAAESAKKGLTVGLIGPDLPFTNNYGVWEDEFKDLGLESCIEHVWKDTVVYLDRNKPIMIGRAYGRVDRDLLHEELLRRCNEAGVTYLNSKVEQIKESPDGHRVVYCGRGHKILCRLAIVASGAASGKLLEYEVGGPRVCVQTAYGVEVEVERYPYDPSLMVFMDYRDCSKEKFTHPEEANPTFLYAMAMSSTRVFFEETCLASKDAMPFDLLKKRLMSRLDAMGVRIIKVYEEEWSYIPVGGSLPNTDQKNLAFGAAASMVHPATGYSVVRSLSEAPRYASVISDILRNRVYSGQYLPGSSEMSSPSMLAWGTLWPQERKRQRSFFLFGLALIIQLDNEGIQTFFESFFRLPKWMWRGFLGSTLSSADLMLFALYMFAIAPNTLRMNLVRHLLSDPTGSAMIRTYLTL

SEQ ID NO: 24 - protein, 536 aa, Triticum aestivum
MESTGAAISAPFGCRALRWAGQRPLRPADARRRRVGPGPGPEQWRSLKASCVATEKPDEKAAPGLGVDFADEEDYVKGGGGELLYVQMQATKAMESQSKIASKLLPIADETSVLDLVIIGCGPAGLSLAAESAKKGLTVGLIGPDLPFTNNYGVWEDEFKDLGLESCIEHVWKDTVVYLDRNKPIMIGRAYGRVDRDLLHEELLRRCNEAGVTYLNSKVEQIKESPDGHRVVYCGRGRKILCRLAIVASGAASGKLLEYEVGGPRVCVQTAYGVEVEVERYPYDPSLMVFMDYRDCFKEKFTHPEEANPTFLYAMAMSSTRVFFEETCLASKDAMPFDLLKKRLMSRLDAMGVRIIKVYEEEWSCIPVGGSLPNTDQKNLAFGAAASMVHPATGYSVVRSLSEAPRYASVISDILRNRVYSGQYLPGSSEMSSPSMLAWGTLWPQERKRQRSFFLFGLALIIQLDNEGIQTFFESFFRLPKWMWRGFLGSTLSSADLMLFALYMFAIAPNTLRMNLVRHLLSDPTGSAMIRTYLTL

SEQ ID NO: 25 - DNA, 1640 bp, Artificial
  RNAi construct
aatgattggccgagcatatggccgggtggaccgagacttgctgcacgaggagttgctgagaagatgcaatgaagctggtgttacatacctcaactcaaaggttgaacagataaaagaatctccagatggacatcgagtggtatattgtggaaggggccgcaagatactttgcaggcttgccattgttgcatctggagcagcatctggtaaacttctagagtatgaggttggaggaccacgtgtttgtgtgcagactgcatacggtgtagaagtcgaggttgaaagatatccgtacgatcccagcttaatggttttcatggactacagggattgtttcaaagagaagttcacacaccctgaggaagccaatccaacatttctctatgccatggccatgtcatctacacgagttttctttgaggaaacatgcttagcttcaaaagatgcaatgccttttgatctccttaagaagaggttgatgtctcggttggatgcgatgggagttcgtatcataaaagtatacgaggaggagtggtcttgtattcctgttggaggatccttaccactagtgaattccaccttcgatacaaggttgtgcctaagtatatcaatgtattttacatgttttgtcatggcagcaccttttggttgtttgccatcatcacctcccttttggttcaatgttcttgctatttacagtgggctcctattcactttggaatcgagttcaaatttcctgtgcagcacattatttcaatcctttcctattagcataaaatgtttccttaggctacctacagtttctttgatgttcacacagaattgatgtgggcatgcattttccatgcataaatcggaacggcatgttctgaaaaattattctaaagataaggacgaatgccaaggaccctgtccatttgttcaaatgtagttctttctctgcataaaacaatttatccttatgaattattcgtattttccccagcattctctgcagtcaagctctgcaatttgtctcacctaacatgtctaatttttaattgcaccaatattgaacagtatatgaatgcactagtggtaaggatcctccaacaggaatacaagaccactcctcctcgtatacttttatgatacgaactcccatcgcatccaaccgagacatcaacctcttcttaaggagatcaaaaggcattgcatcttttgaagctaagcatgtttcctcaaagaaaactcgtgtagatgacatggccatggcatagagaaatgttggattggcttcctcagggtgtgtgaacttctctttgaaacaatccctgtagtccatgaaaaccattaagctgggatcgtacggatatctttcaacctcgacttctacaccgtatgcagtctgcacacaaacacgtggtcctccaacctcatactctagaagtttaccagatgctgctccagatgcaacaatggcaagcctgcaaagtatcttgcggccccttccacaatataccactcgatgtccatctggagattcttttatctgttcaacctttgagttgaggtatgtaacaccagcttcattgcatcttctcagcaactcctcgtgcagcaagtctcggtccacccggccatatgctcggccaatcatt

SEQ ID NO: 26 - DNA, 19 bp, Artificial
  Sense strand of siRNA
gcuguugccuauagcugau

SEQ ID NO: 27 - RNA, 19 nt, Artificial
  Sense strand of siRNA
agaaaggacucacuguugg

SEQ ID NO: 28 - RNA, 19 nt, Artificial
  Sense strand of siRNA
caagccgauaaugauuggc

SEQ ID NO: 29 - RNA, 19 nt, Artificial
  Sense strand of siRNA
ggggccacaagauacuuug

SEQ ID NO: 30 - RNA, 19 nt, Artificial
  Sense strand of siRNA
guauacgaggaggaguggu

SEQ ID NO: 31 - DNA, 20 bp, Artificial
  Oligonucleotide primer
gatgacatggccatggcata

SEQ ID NO: 32 - DNA, 22 bp, Artificial
  Oligonucleotide primer
caccttcgatacaaggttgtgc

SEQ ID NO: 33 - DNA, 22 bp, Artificial
  Oligonucleotide primer
acaaattgcagagcttgactgc

SEQ ID NO: 34 - DNA, 18 bp, Artificial
  Oligonucleotide primer
agtacgccaagaccttct

SEQ ID NO: 35 - DNA, 18 bp, Artificial
  Oligonucleotide primer
gtgaagttgttgtagtcg

SEQ ID NO: 36 - DNA, 18 bp, Artificial
  Oligonucleotide primer
gggaggatgaattcaaag

SEQ ID NO: 37 - DNA, 18 bp, Artificial
  Oligonucleotide primer
ccacatccatttgggcaa

SEQ ID NO: 38 - DNA, 20 bp, Artificial
  Oligonucleotide primer
cattgttgcatctggagcag

SEQ ID NO: 39 - DNA, 19 bp, Artificial
  Oligonucleotide primer
tgatgcagccctctcagat

SEQ ID NO: 40 - DNA, 19 bp, Artificial
  Oligonucleotide primer
ggacgcagccctctcatac

SEQ ID NO: 41 - DNA, 19 bp, Artificial
  Oligonucleotide primer
tgacgcagccctctcagac

SEQ ID NO: 42 - DNA, 19 bp, Artificial
  Oligonucleotide primer
tagagctcgtcaaaggtcc

SEQ ID NO: 43 - DNA, 19 bp, Artificial
  Oligonucleotide primer
taaagctcgtcaaaggtca

SEQ ID NO: 44 - DNA, 19 bp, Artificial
  Oligonucleotide primer
taaagctcgtcaaaggtcc

SEQ ID NO: 45 - DNA, 20 bp, Artificial
  Oligonucleotide primer
gtatattgtggaaggggcca

SEQ ID NO: 46 - DNA, 20 bp, Artificial
  Oligonucleotide primer
gtatattgtgaaagggaccg

SEQ ID NO: 47 - DNA, 20 bp, Artificial
  Oligonucleotide primer
gtatattgtggaaggggccg

SEQ ID NO: 48 - DNA, 20 bp, Artificial
  Oligonucleotide primer
aaaaccattaagctgggatc

SEQ ID NO: 49 - DNA, 20 bp, Artificial
  Oligonucleotide primer
aaaaccattaagctggggtt

SEQ ID NO: 50 - DNA, 20 bp, Artificial
  Oligonucleotide primer
gaactggtgcagaaacatcc

SEQ ID NO: 51 - DNA, 27 bp, Artificial
  Oligonucleotide primer
caagaacggaaacgtcagcgctcattc

SEQ ID NO: 52 - DNA, 28 bp, Artificial
  Oligonucleotide primer
ctttggattggccttgataattcaactg

SEQ ID NO: 53 - DNA, 28 bp, Artificial
  Oligonucleotide primer
gctcggccaatcattatcggcttgttac

SEQ ID NO: 54 - DNA, 28 bp, Artificial
  Oligonucleotide primer
acacgacagtatccttccatacatgctc

SEQ ID NO: 55 - DNA, 27 bp, Artificial
  Oligonucleotide primer
gcaatgatcaggacctacctgaccttg

SEQ ID NO: 56 - DNA, 28 bp, Artificial
  Oligonucleotide primer
gtctgcgagaaatctagaaagtgtacag

SEQ ID NO: 57 - DNA, 30 bp, Artificial
  Oligonucleotide primer
taatcaactacttgaaggatgctaccagac

SEQ ID NO: 58 - DNA, 30 bp, Artificial
  Oligonucleotide primer
gaagtttcatcagctataggcaacagctgc

SEQ ID NO: 59 - DNA, 22 bp, Artificial
  Oligonucleotide primer
ggagatcgtacagaccgagcgg

SEQ ID NO: 60 - DNA, 23 bp, Artificial
  Oligonucleotide primer
catcagctataggcaacagctgc

SEQ ID NO: 61 - DNA, 20 bp, Artificial
  Oligonucleotide primer
atacgaactcccatcgcatc

SEQ ID NO: 62 - DNA, 23 bp, Artificial
  Oligonucleotide primer
gctttttccggttgcccaaatgg

SEQ ID NO: 63 - DNA, 21 bp, Artificial
  Oligonucleotide primer
ggtgctcatcgggactcaagg

SEQ ID NO: 64 - DNA, 22 bp, Artificial
  Oligonucleotide primer
taatcgccgtctgccacgtgcc

SEQ ID NO: 65 - DNA, 33 bp, Artificial
  Oligonucleotide primer
cccgggggtaccaatgattggccgagcatatgg

SEQ ID NO: 66 - DNA, 34 bp, Artificial
  Oligonucleotide primer
ggcgcgccactagtggtaaggatcctccaacagg

SEQ ID NO: 67 - DNA, 33 bp, Artificial
  Oligonucleotide primer
ctcgaggctagcaatgattggccgagcatatgg

SEQ ID NO: 68 - DNA, 32 bp, Artificial
  Oligonucleotide primer
gaattcactagtggtaaggatcctccaacagg

SEQ ID NO: 69 - DNA, 20 bp, Artificial
  Oligonucleotide primer
atgatggcatatgcagcagc